(19) United States
(12) Patent Application Publication
(10) Pub. No.: US 2017/0057955 A1
(43) Pub. Date: Mar. 2, 2017

(54) Pyridopyrimidinone Compounds for Modulating the Catalytic Activity of Histone Lysine Demethylases (KDMs)

(71) Applicant: Dong-A Socio Holdings Co., Ltd., Seoul (KR)

(72) Inventors: Myeong-seop KIM, Suwon (KR); Taesun Park, Hwaseong (KR); Taeyoung Yoon, Seoul (KR); Seung Min Yang, Yongin (KR); Hae-Sun Kim, Hwaseong (KR); Jun Gyu Kim, Seoul (KR)

(21) Appl. No.: 15/246,721

(22) Filed: Aug. 25, 2016

Related U.S. Application Data
(60) Provisional application No. 62/209,923, filed on Aug. 26, 2015.

(51) Int. Cl. C07D 471/04

(52) U.S. Cl. CPC C07D 471/04

(57) ABSTRACT

The present invention provides a compound of formula (I) being capable of modulating the activity of histone lysine demethylase (KDM), pharmaceutical compositions thereof, methods to prepare the said compounds, and the use of such compounds as a medicament. The compound of formula (I) acts as KDM inhibitor with marked potency, thereby having an outstanding potential for a pharmaceutical intervention of cancer and any other diseases related to KDM dysregulation.

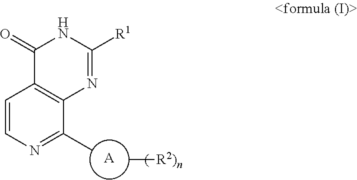

### CROSS REFERENCE TO RELATED APPLICATION

This application claims the benefit of U.S. Provisional Application Ser. No. 62/209,923, filed on Aug. 26, 2015, the contents of which are incorporated by reference herein in their entireties.

### TECHNICAL FIELD

The present invention provides compounds that are capable of modulating the activity of histone lysine demethylase (KDM), pharmaceutical compositions thereof, methods to prepare the said compounds, and the use of such compounds as a medicament.

### BACKGROUND

The nucleosome is a basic unit to build up the extremely complicating chromatin structure inside the cells of eukaryotes. In the nucleosome, the genomic DNA is wrapped around a histone octamer which is composed of two copies of four different core histone subunits, H2A, H2B, H3 and H4. Mutations in the genomic DNA sequence could cause aberrant expressions of essential proteins that are required to maintain homeostasis of life, leading to serious illness such as birth defects, diabetes, neurological disorders and cancer. However, it has been shown for the past few decades that, even without the sequence alterations, the production and biological functions of the proteins can also be perturbed by newly termed “epigenetic” changes in the genomic DNA and histones. DNA methylation and histone post-translational modifications are two major epigenetic events that are commonly occurred in most of living organisms. In humans, about 70% of cytosines within CpG dinucleotides are usually methylated and the N-terminal tails of the histones are subjected to several covalent modifications including methylation, acetylation, phophorylation, ubiquitination and sumoylation (Shilatifard, A. Annu. Rev. Biochem. (2006) 75, 243-269). At some specific lysine residues of the histones, one, two or three methyl groups can be added or removed by two distinct classes of enzymes, histone methytransferases (HMTs) and histone demethylases (KDMs), respectively. These methylation states play an essential role in regulating gene expression in a context-dependent manner. For instance, di/tri methylation on the lysine 4, 36 or 79 of the histone H3 is generally attributed to an active mark for gene expression. In contrast, di/tri methylation on the lysine 9, 27 of the H3 is usually linked with a closed chromatin conformation (heterochromatin), leading to repression of gene expression (Martin, C. & Zhang, Y. Nat. Rev. Mol. Cell Biol. (2005) 6, 838-849).

More than 30 KDMs have been found in mammals and KDMs can be classified into two families based on the underlying mechanism by which they remove methyl groups from the histone tails; LSD1 (lysine specific demethylase 1) and JmjC-containing KDMs. LSD1, the first hisone demethylase discovered in 2004, erases mono- and di-methyl marks on the H3K4 and H3K9 using flavin as a cofactor. As a protonated amine in the substrate is required for its demethylation pathway, LSD1 cannot act on tri-methylated lysines. The existence of different class of KDMs that could remove a trimethyl mark was, therefore, predicted and identified later as catalytic JmjC-domain containing proteins. Compared with LSD1, these proteins contain much more diverse subfamilies including KDM2, KDM3, KDM4, KDM5, KDM6 and PHF2/8, all of which utilize Fe(II) and alpha-ketoglutarate (αKG) as cofactors (Spannhoff, A. et al. Chem Med Chem (2009) 4, 1568-1582)

Many studies have shown that KDMs are implicated in the etiology of cancer, one of the most devastatinghuman diseases (Cloos, P. A. et al. Genes Dev. (2008) 22, 1115-1140). LSD1 is overexpressed in various types of cancer cells including prostate, lung and breast cancer in which LSD1 may enhance oncogenic properties of the cells by modulating the expression of pro-survival genes and tumor suppressor genes such as p53 (Scoumanne, A. & Chen X. J. Biol. Chem. (2007) 282, 15471-15475)

Small hairpin RNA (shRNA)-mediated depletion of KDM2B (also known as FBXL10) attenuated the growth of acute myeloid leukemia (AML) cell line, in which KDM2B is overexpressed (He, J. et al. Blood (2011) 117, 3869-3880). Alterations in the expression of Polycomb target genes may account for this anti-proliferative effect on the basis of a recent finding that KDM2B regulated the expression (Tzatsos, A. et al. J. Clin. Invest. (2013) 123, 727-739).

Initially identified as a putative oncogene GASC1 (gene amplified in squamous cell carcinoma 1), KDM4C, which removes di- and tri-methyl marks from H3K9 as well as H3K36, is genomically amplified in breast carcinoma and prostate carcinoma and required for the growth of these malignant cells (Liu, G. et al. Oncogene (2009) 28, 4491-4500; Wissmann, M. et al. Nature Cell Biol. (2007) 9, 347-353).

The family of KDM5/JARID1 (Jumonji AT-rich interactive domain 1) in human comprises four members, KDM5A/RBP2, KDM5B/PLU-1, KDM5C/SMCX and KDM5D/SMCY, which share highly conserved structural motifs that include a JmjN domain, a catalytic JmjC domain, an ARID DNA binding domain, a zinc finger and two to three PHD (plant homeodomain) fingers. These subfamily members are shown to be involved in the pathogenesis of cancer.

Aberrantly high expression of KDM5A is often found in gastric and cervical cancers (Zeng, J. et al. Gastroenterology (2010), 138, 981-992; Hidalgo, A. et al. BMC Cancer (2005) 5, 77), and KDM5B is also up-regulated in several malignancies such as breast, prostate, lung cancers and melanoma (Lu, P. J. et al. J. Biol. Chem. (1999) 274, 15633-15645; Xiang, Y. et al. Proc. Natl. Acad. Sci. USA (2007) 104, 19226-19231; Hayami, S. et al. Mol. Cancer (2010) 9, 59; zur Hausen, H. Virilogy (2009) 384, 260-265). Acquired drug resistance in cancer is often linked to the presence of cancer stem cells which are capable of reforming tumor cells. Recent studies demonstrated that, in drug-resistant lung cancer cells and melanoma cells, disruption of KDM5A or KDM5B enzymatic function by RNA interference reduced the cancer stem cell-like properties and increased drug sensitivity, thus exerting an anti-proliferative effect on those cells (Sharma, S. et al. Cell (2010) 141, 69-80; Roesch, A. et al. Cancer Cell (2013) 23, 811-825).

KDM5C seems to be associated with mental retardation and some forms of cancer. Gene expression analysis for clear cell renal cell carcinoma (ccRCC) revealed that truncation mutation of KDM5C was found in 3% of ccRCC tumors and most of the mutation was occurred concomitantly with VHL (Von Hippel-Lindau tumor suppressor) mutations (Dalgliesh, G. L. et al. Nature (2010) 463, 360-363).

Although direct linkage between KDM5D and cancer has yet to be known, one study shows that 52% of tested prostate cancer cases contain deletion of KDM5D gene, implying an association of KDM5D with the disease (Perinchery, G. et al. J. Urol. (2000) 163, 1339-1342).

Similar to KDM5A and KDM5B, KDM7B (also known as PHF8) enzymatically active on H3K9me1/2 and H4K20me1 is exhibited to govern an anti-cancer drug (retinoic acid) response in acute promyelocytic leukemia (Arteaga, M. F. et al. Cancer Cell (2013) 23, 376-389).

Taken together, deregulation of KDM is involved in initiation, maintenance, progression and other pathogenesis of cancer, suggesting KDM is a very promising therapeutic target for the intervention of the disease. The present invention is directed to KDM inhibitory compounds with marked potency, thereby having an outstanding potential for a pharmaceutical intervention of cancer and any other diseases related to KDM dysregulation.

### INFORMATION DISCLOSURE

Several KDM-inhibitory compounds have been previously described in the publications WO 2014/055634, US 2014/0371214, WO 2014/053491, WO 2014/139326, WO 2014/151106, WO 2014/164708 and WO 2015/035062, but they are chemically and structurally different compounds from the present invention.

### SUMMARY OF THE INVENTION

Accordingly, a first aspect of the present invention relates to a compound of the formula (I)

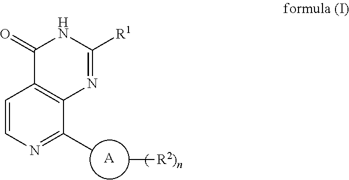

wherein:
R¹ is selected from H, halogen, alkyl, alkoxy, alkenyl, carbocyclyl, aryl, heterocyclyl, heteroaryl, —C(O)R^{b}, —C(O)OR^{b} and —C(O)N(R^{b})₂, —CH₂—O-aryl, —CH₂—O-biaryl, wherein each alkyl, alkenyl, carbocyclyl, aryl, heterocyclyl, heteroaryl, —CH₂—O-aryl or —CH₂—O-biaryl of R¹ is optionally substituted with one or more R^{x};
A is heteroaryl that is substituted with one or more R², wherein heteroaryl is monocyclic or bicyclic ring; and n is from 1 to 6.

R² is independently selected from H, halogen, alkyl, alkoxy, alkenyl, carbocyclyl, aryl, heterocyclyl, heteroaryl, —OR^{b}, —SR^{b}, —N(R^{b})₂, —NR^{b}C(O)R^{b}, —NHC(O)OR^{b}, —NHC(O)NHR^{b}, —C(O)R^{b}, —C(O)OR^{b}, —C(O)N(R^{b})₂, wherein each alkyl, alkenyl, carbocyclyl, aryl, heterocyclyl or heteroaryl of R² is optionally substituted with one or more R^{x};

R^{b} is selected from H, alkyl, alkenyl, carbocyclyl, aryl, heterocyclyl and heteroaryl, wherein each alkyl, alkenyl, carbocyclyl, and heterocyclyl is optionally substituted with one or more R^{x};
each R^{x} is selected from halogen, alkyl, alkenyl, carbocyclyl, aryl, heterocyclyl, heteroaryl, —NO₂, —N(R^{y})₂, —CH₂—N(R^{y})₂, —CN, —C(O)—N(R^{y})₂, —S(O)—N(R^{y})₂, —S(O)₂—N(R^{y})₂, —O-aryl, —O-heteroaryl, —O—R^{y}, —S—R^{y}, —O—C(O)—R^{y}, —O—C(O)—O—R^{y}, —C(O)—R^{y}, —C(O)—O—R^{y}, —S(O)—R^{y}, —S(O)₂—R^{y}, —O—C(O)—N(R^{y})₂, —N(R^{y})—C(O)—OR^{y}, —N(R^{y})—S(O)—N(R^{y})₂, and —N(R^{y})—S(O)₂—N(R^{y})₂, wherein alkyl, alkenyl, carbocyclyl, aryl, heterocyclyl or heteroaryl of R^{x} is optionally substituted with one or more groups independently selected from the group consisting of halogen, alkyl, alkoxy, hydroxy, and aryl;
each R^{y} is selected from H, alkyl, alkenyl, carbocyclyl, aryl, heterocyclyl, heteroaryl, —C(O)R^{z}, —C(O)OR^{z}, —C(O)N(R^{z})₂, —CH₂—R^{z}, that is optionally substituted with one or more groups independently selected from the group consisting of halogen, alkyl, carbocyclyl and heterocyclyl;
each R^{z} is selected from carbocyclyl, aryl, heterocyclyl, heteroaryl, alkylcarbocyclyl, alkylheterocyclyl, wherein each carbocyclyl, aryl, heterocyclyl, heteroaryl, alkylcarbocyclyl, alkylheterocyclyl is optionally substituted with one or more groups independently selected from the group consisting of alkyl, alkoxy, hydroxy, hydroxyalkyl, amino.

In another aspect, the present invention relates to pharmaceutical compositions comprising at least one compound of formula (I) as defined herein.

A further aspect of the present invention relates to a compound of formula (I) as defined herein for use as a medicament.

A further aspect of the present invention relates to a compound of formula (I) as defined herein for use in the prevention or treatment of a HDME related diseases, such as cancers.

A further aspect of the present invention relates to a compound of formula (I) as defined herein for use in the preparation of a pharmaceutical composition for the treatment of HDME related diseases, such as cancers.

In additional aspect, the present invention provides a method for preventing or treating HDME related diseases in a subject, and the said method comprises administrating to a said subject a therapeutically effective amount of at least one compound of formula (I) as defined herein.

### BRIEF DESCRIPTION OF THE DRAWINGS

### Detailed Description

The term ‘halogen’ as used herein refers to fluorine, chlorine, bromine or iodine.

The term ‘alkyl’ as used herein refers to a straight chain or branched chain hydrocarbon residue, unless otherwise stated. The examples of the C₁₋₈alkyl include methyl, ethyl, propyl, isopropyl, butyl, isobutyl, pentyl, hexyl, heptyl, octyl and the like.

The term ‘alkenyl’ as used herein denotes a monovalent group derived from a straight- or branched-chain aliphatic moiety having at least one carbon-carbon double bond by the removal of a single hydrogen atom. In certain embodiments, alkenyl contains 2-6 carbon atoms. In certain embodiments, alkenyl contains 2-5 carbon atoms. In certain embodiments, alkenyl contains 2-4 carbon atoms. In another embodiment, alkenyl contains 2-3 carbon atoms. Alkenyl groups include, for example, vinyl, allyl, butenyl, 1-methyl-2-buten-1-yl, and the like.

The term ‘alkoxy’ as used herein includes an alkyl-oxygen radical having alkyl as defined above, unless otherwise stated. The examples of the C₁₋₈alkoxy include methoxy, ethoxy, propoxy, butoxy, pentoxy, and the like.

The term ‘carbocyclyl’ as used herein refers to a saturated or partially unsaturated cyclic aliphatic monocyclic or bicyclic ring systems, as described herein, having from 3 to 10 members, wherein the aliphatic ring system is optionally substituted as defined above and described herein.

The term ‘heterocycle’ or ‘heterocyclyl’ as used herein refers to a 4 to 13 membered non-aromatic compound including 1 to 3 hetero atoms selected from the group consisting of N, O and S, unless otherwise stated.

The term ‘heteroaryl’ as used herein refers to a 4 to 13 membered heteroaromatic compound including 1 to 3 hetero atoms selected from the group consisting of N, O and S, unless otherwise stated.

The term ‘aryl’ as used herein refers to a C₆₋₁₂ aromatic compound, unless otherwise stated.

In a preferred embodiment of the present invention, R¹ is selected from H, halogen, C₁₋₆alkyl, C₁₋₆alkoxy, C₂₋₆alkenyl, C₃₋₈carbocyclyl, C₆₋₁₀aryl, 4- to 8-membered heterocyclyl including 1 to 3 hetero atoms selected from the group consisting of N, O and S, 4- to 8-membered heteroaryl including 1 to 3 hetero atoms selected from the group consisting of N, O and S, —C(O)R^{b}, —C(O)OR^{b} and —C(O)N(R^{b})₂, —CH₂—O—C₆₋₁₀aryl, —CH₂—O-bi-C₆₋₁₀ aryl, wherein each C₁₋₆alkyl, C₂₋₆alkenyl, C₃₋₈carbocyclyl, C₆₋₁₀aryl, 4- to 8-membered heterocyclyl including 1 to 3 hetero atoms selected from the group consisting of N, O and S, 4- to 8-membered heteroaryl including 1 to 3 hetero atoms selected from the group consisting of N, O and S, —CH₂—O—C₆₋₁₀aryl or —CH₂—O-bi-C₆₋₁₀aryl of R¹ is optionally substituted with one or more R^{x}.

In a more preferred embodiment of the present invention, R¹ is selected from H, F, Cl, methyl, ethyl, propyl, isopropyl, butyl, isobutyl, n-butyl, ethenyl, benzyl, CH₂O-phenyl, CH₂O-biphenyl and C(O)OCH₃, wherein each methyl, ethyl, propyl, isopropyl, butyl, isobutyl, n-butyl, ethenyl, benzyl, CH₂O-phenyl and CH₂O-biphenyl is optionally substituted with one or two groups selected from H, F, Cl, cyclopentyl, phenyl, benzyl, 2-phenylpropyl and benzoyl.

In a preferred embodiment of the present invention, A is 4- to 8-membered heteroaryl including 1 to 3 hetero atoms selected from the group consisting of N, O and S, that is substituted with one or more R², wherein heteroaryl is monocyclic or bicyclic ring; and n is from 1 to 4.

In a more preferred embodiment of the present invention, A is pyrazole, imidazole, thiazole, oxazole, thiadiazole, oxadiazole, triazole, dithiazole, dioxazole, pyrimidine, pyrazine or pyridazine that is substituted with one or more R²; and n is from 1 to 2.

In a preferred embodiment of the present invention, R² is independently selected from H, F, Cl, Br, C₁₋₆alkyl, C₁₋₆alkoxy, C₂₋₆alkenyl, C₃₋₈carbocyclyl, C₆₋₁₀aryl, 4- to 8-membered heterocyclyl including 1 to 3 hetero atoms selected from the group consisting of N, O and S, 4- to 8-membered heteroaryl including 1 to 3 hetero atoms selected from the group consisting of N, O and S, —OR^{b}, —SR^{b}, —N(R^{b})₂, —NR^{b}C(O)R^{b}, —NHC(O)OR^{b}, —NHC(O)NHR^{b}, —C(O)R^{b}, —C(O)OR^{b}, —C(O)N(R^{b})₂, wherein each C₁₋₆ alkyl, C₁₋₆alkoxy, C₂₋₆alkenyl, carbocyclyl, C₆₋₁₀ aryl, 4- to 8-membered heterocyclyl including 1 to 3 hetero atoms selected from the group consisting of N, O and S or 4- to 8-membered heteroaryl including 1 to 3 hetero atoms selected from the group consisting of N, O and S of R² is optionally substituted with one or more R^{x}.

In a more preferred embodiment of the present invention, R² is independently selected from H, F, Cl, Br, C₁₋₃alkyl, C₁₋₃alkoxy, C₂₋₄alkenyl, cyclopentyl, cyclohexyl, phenyl, pyridine, pyrazole, imidazole, oxazole, pyrimidine, piperidine, piperazine, pyrrolidine, morpholine, —OR^{b}, —SR^{b}, —N(R^{b})₂, —NR^{b}C(O)R^{b}, —NHC(O)OR^{b}, —NHC(O)NHR^{b}, —C(O)R^{b}, —C(O)OR^{b}, —C(O)N(R^{b})₂, wherein each C₁₋₃alkyl, C₁₋₃alkoxy, C₂₋₄alkenyl, phenyl, pyrazole, pyrimidine, piperidine and morpholine is optionally substituted with one or more R^{x}.

In a still more embodiment of the present invention, R² is independently selected from the group consisting of H, methyl, chloro, tolyl, methylpiperazinylmethylphenyl, hydroxymethylphenyl, fluorophenyl, chlorophenyl, benzyloxyphenyl, hydroxypropylphenyl, methoxybenzyloxyphenyl, phenoxyphenyl, methylpyrazolyl, pyridinyl, benzylpyrazolyl, piperazinylpyridinyl, piperazinylphenyl, morpholinocarbonylphenyl, piperidinlypyrazolyl, acetylphenyl, hydroxyphenyl, hydroxyethylpyrazolyl, pyrrolidinylethylpyrazolyl, morpholinoethylpyrazolyl, methylpiperazinylethylpyrazolyl, methylpiperazinylphenyl, methylpiperazinylbenzyl, pyrrolidinylmethylphenyl, cylopentylpyrazolyl, methylcarbonylaminophenyl, amino, hydroxyethylamino, hydroxypropylamino, dihydroxypropylamino, dihydroxypropanylamino, hydroxybutylamino, tetrahydrofuranylamino, tetrahydropyranylamino, methoxyethylamino, dimethylaminoethylamino, dimethylaminopropylamino, aminoethylamino, methylpiperazinylethylamino, methylpyrrolidinylmethylamino, methylpyrrolidinylethylamino, piperidinylamino, methylpiperidinylamino, isopropylpiperidinylmethylamino, aminocyclohexylamino, hydroxybutylamino, methylmorpholinylmethyl amino, hydroxyethylpiperidinylmethylamino, benzylpiperidinylamino, ethoxycarbonyl, hydroxyethyl, benzyl, morpholinoethyl, benzylpiperidinylmethylamino, methoxyethylpiperidinylmethylamino, cyclopentylpiperidinylmethylamino, allylamino, phenylamino, morpholinyl, morpholinoethylamino, tetrahydrofuranylamino, diethylamino, benzylpyrrolidinylamino, dichlorophenethylamino, chlorophenylpropylamino, phenyoxyphenethylamino, hydroxy(pyridinyl)ethylamino, dimethylphenethylethylamino, hydroxy(piperazinyl)propylamino, cyclohexylmethylamino, diethylaminopentylamino, pyrrolidinylbutylamino, diisopropylpentylamino, hydroxypentanylamino, ethoxycarbonyl, phenoxypropylamino and benzoylamino.

In a preferred embodiment of the present invention, R^{b} is selected from the group consisting of H, C₁₋₆alkyl, C₂₋₆alkenyl, C₃₋₈carbocyclyl, C₆₋₁₀aryl, 4- to 8-membered heterocyclyl including 1 to 3 hetero atoms selected from the group consisting of N, O and S and 4- to 8-membered heteroaryl including 1 to 3 hetero atoms selected from the group consisting of N, O and S, wherein each C₁₋₆alkyl, C₂₋₆alkenyl, C₃₋₈carbocyclyl, C₆₋₁₀ aryl, 4- to 8-membered heterocyclyl including 1 to 3 hetero atoms selected from the group consisting of N, O and S and 4- to 8-membered heteroaryl including 1 to 3 hetero atoms selected from the group consisting of N, O and S is optionally substituted with one or more R^{x}.

In a more preferred embodiment of the present invention, R^{b} is selected from the group consisting of H, methyl, ethyl, propyl, butyl, pentyl, propenyl, phenyl, pyrrole, pipeazine, piperidine and cyclohexyl, wherein each H, methyl, ethyl, propyl, butyl, pentyl, propenyl, phenyl, pyrrole, pipeazine, piperidine and cyclohexyl is optionally substituted with one or more R^{x}.

In a preferred embodiment of the present invention, R^{x} is selected from halogen, C₁₋₆ alkyl, C₂₋₆alkenyl, C₃₋₈carbocyclyl, C₆₋₁₀aryl, heteroaryl, heterocyclyl, —NO₂, —N(R^{y})₂, —CH₂—N(R^{y})₂, —CN, —C(O)—N(R^{y})₂, —S(O)—N(R^{y})₂, —S(O)₂—N(R^{y})₂, —O—C₆₋₁₀aryl, —O-heteroaryl, —O—R^{y}, —S—R^{y}, —O—C(O)—R^{y}, —O—C(O)—O—R^{y}, —C(O)—R^{y}, —C(O)—O—R^{y}, —S(O)—R^{y}, —S(O)₂—R^{y}, —O—C(O)—N(R^{y})₂, —N(R^{y})—C(O)—OR^{y}, —N(R^{y})—S(O)—N(R^{y})₂, and —N(R^{y})—S(O)₂—N(R^{y})₂, wherein alkyl, alkenyl, carbocyclyl, aryl, heterocyclyl or heteroaryl of R^{x} is optionally substituted with one or more groups independently selected from halogen, alkyl, alkoxy and hydroxy; and heteroaryl, and heterocyclyl are independently 4- to 8-membered ring including 1 to 3 hetero atoms selected from the group consisting of N, O and S.

In a more preferred embodiment of the present invention, R^{x} is selected from F, Cl, Br, OH, methyl, ethyl, propyl, cyclopentyl, cyclohexyl, phenyl, —C(O)—R^{y}, —N(R^{y})₂, wherein methyl, ethyl, propyl, cyclopentyl, cyclohexyl or phenyl, of R^{x} is optionally substituted with one or more groups independently selected from F, Cl, Br, C₁₋₄alkyl, C₁₋₄alkoxy, and hydroxy.

In a preferred embodiment, R^{y} is selected from the group consisting of H, C₁₋₄alkyl, C₂₋₆alkenyl, C₃₋₈carbocyclyl, C₆₋₁₀aryl, 4- to 8-membered heterocyclyl including 1 to 3 hetero atoms selected from the group consisting of N, O and S and 4- to 8-membered heteroaryl including 1 to 3 hetero atoms selected from the group consisting of N, O and S, —C(O)R^{z}, —C(O)OR^{z}, —C(O)N(R^{z})₂, —CH₂—R^{z}, that is optionally substituted with one or more groups independently selected from F, Cl, Br, C₁₋₄alkyl, C₃₋₈carbocyclyl and 4- to 8-membered heterocyclyl including 1 to 3 hetero atoms selected from the group consisting of N, O and S.

In a more preferred embodiment, R^{y} is selected from the group consisting of H, methyl, ethyl, isopropyl, cyclopentyl, phenyl, —C(O)R^{z}, —C(O)OR^{z}, —C(O)N(R^{z})₂, and —CH₂—R^{z}, that is optionally substituted with one or more groups independently selected from F, Cl, Br, hydroxyl, methoxy, methyl and ethyl.

In a preferred embodiment, R^{z} is selected from the group consisting of C₃₋₈ carbocyclyl, C₆₋₉ aryl, heterocyclyl, heteroaryl, C₁₋₄ alkyl C₃₋₈ carbocyclyl, C₁₋₄ alkylheterocyclyl, wherein each C₃₋₈ carbocyclyl, C₆₋₉ aryl, heterocyclyl, heteroaryl, C₁₋₄ alkyl C₃₋₈ carbocyclyl and C₁₋₄ alkylheterocyclyl is optionally substituted with one or more groups independently selected from C₁₋₄ alkyl, C₁₋₄ alkoxy, hydroxy, hydroxy C₁₋₄ alkyl and amino, and heteroaryl, and heterocyclyl are independently 4- to 8-membered ring including 1 to 3 hetero atoms selected from the group consisting of N, O and S.

In a more preferred embodiment, R^{z} is selected from the group consisting of phenyl and piperazine, which is optionally substituted with one or more groups independently selected from C₁₋₄ alkyl, C₁₋₄ alkoxy, hydroxy, hydroxy C₁₋₄ alkyl and amino.

In a far more preferred embodiment of the present invention, the compound represented by the above formula (I) may be selected from the group consisting of the compounds shown in Table 1 below.

TABLE 1

| | | | Synthetic | | |
|---|---|---|---|---|---|
| No. | Chemical Structure | Chemical Name | Scheme | ¹H NMR | MS |
| 1 | 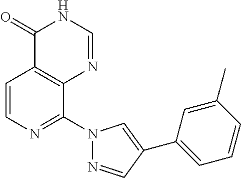 [SMILES: Cc1cccc(-c2cnn(-c3nccc4c(=O)[nH]cnc34)c2)c1] | 8-(4-(m-tolyl)-1H-pyrazol- 1-yl)pyrido[3,4- d]pyrimidin-4(3H)-one | Scheme A | none | 304 |
| 2 | 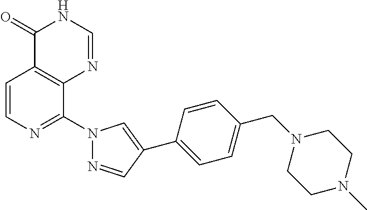 [SMILES: CN1CCN(Cc2ccc(-c3cnn(-c4nccc5c(=O)[nH]cnc45)c3)cc2)CC1] | 8-(4-(4-((4- methylpiperazin-1- yl)methyl)phenyl)-1H- pyrazol-1-yl)pyrido[3,4- d]pyrimidin-4(3H)-one | Scheme A | none | 401 |
| 3 | 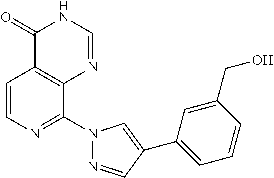 [SMILES: O=c1[nH]cnc2c(-n3cc(-c4cccc(CO)c4)cn3)nccc12] | 8-(4-(3- (hydroxymethyl)phenyl)- 1H-pyrazol-1- yl)pyrido[3,4-d]pyrimidin- 4(3H)-one | Scheme A | none | 319 |
| 4 | 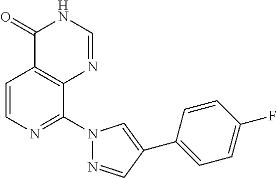 [SMILES: O=c1[nH]cnc2c(-n3cc(-c4ccc(F)cc4)cn3)nccc12] | 8-(4-(4-fluorophenyl)-1H- pyrazol-1-yl)pyrido[3,4- d]pyrimidin-4(3H)-one | Scheme A | ¹H NMR (400 MHz, DMSO) δ 8.91 (s, 1H), 8.59 (d, J = 4.70 Hz, 1H), 8.30 (s, 1H), 8.27 (s, 1H), 8.04 (d, J = 5.09 Hz, 1H), 7.73 (m, 2H), 7.47 (s, 1H), 7.23 (t, 1H) | 307 |
| 5 | 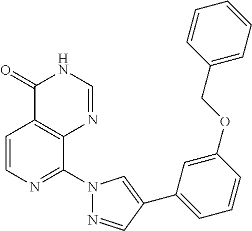 [SMILES: O=c1[nH]cnc2c(-n3cc(-c4cccc(OCc5ccccc5)c4)cn3)nccc12] | 8-(4-(3- (benzyloxy)phenyl)-1H- pyrazol-1-yl)pyrido[3,4- d]pyrimidin-4(3H)-one | Scheme A | none | 395 |
| 6 | 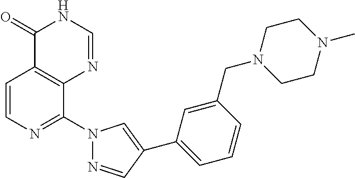 [SMILES: CN1CCN(Cc2cccc(-c3cnn(-c4nccc5c(=O)[nH]cnc45)c3)c2)CC1] | 8-(4-(3-((4- methylpiperazin-1- yl)methyl)phenyl)-1H- pyrazol-1-yl)pyrido[3,4- d]pyrimidin-4(3H)-one | Scheme A | none | 401 |
| 7 | 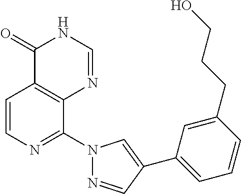 [SMILES: O=c1[nH]cnc2c(-n3cc(-c4cccc(CCCO)c4)cn3)nccc12] | 8-(4-(3-(3- hydroxypropyl)phenyl)- 1H-pyrazol-1- yl)pyrido[3,4-d]pyrimidin- 4(3H)-one | Scheme A | none | 347 |
| 8 | 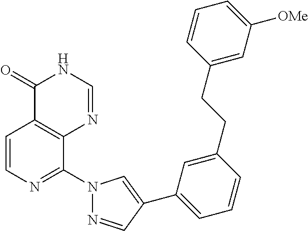 [SMILES: COc1cccc(CCc2cccc(-c3cnn(-c4nccc5c(=O)[nH]cnc45)c3)c2)c1] | 8-(4-(3-((3- methoxybenzyl)oxy)phenyl)- 1H-pyrazol-1- yl)pyrido[3,4-d]pyrimidin- 4(3H)-one | Scheme A | none | 425 |
| 9 | 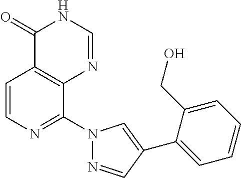 [SMILES: O=c1[nH]cnc2c(-n3cc(-c4ccccc4CO)cn3)nccc12] | 8-(4-(2- (hydroxymethyl)phenyl)- 1H-pyrazol-1- yl)pyrido[3,4-d]pyrimidin- 4(3H)-one | Scheme A | none | 319 |
| 10 | 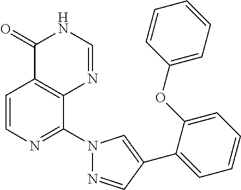 [SMILES: O=c1[nH]cnc2c(-n3cc(-c4ccccc4Oc4ccccc4)cn3)nccc12] | 8-(4-(2-phenoxyphenyl)- 1H-pyrazol-1- yl)pyrido[3,4-d]pyrimidin- 4(3H)-one | Scheme A | none | 381 |
| 11 | 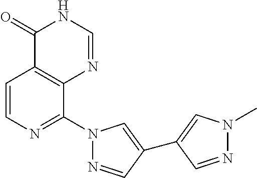 [SMILES: Cn1cc(-c2cnn(-c3nccc4c(=O)[nH]cnc34)c2)cn1] | 8-(1′-methyl-1H,1′H-[4,4′- bipyrazol]-1- yl)pyrido[3,4-d]pyrimidin- 4(3H)-one | Scheme A | ¹H NMR (400 MHz, DMSO) δ 9.02 (s, 1H), 8.57 (br s, 1H), 8.25 (br s, 1H), 8.04 (m, 1H), 7.88 (s, 1H), 7.84 (s, 1H), 7.74 (s, 1H), 3.93 (s, 3H) | 293 |
| 12 | 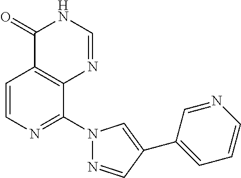 [SMILES: O=c1[nH]cnc2c(-n3cc(-c4cccnc4)cn3)nccc12] | 8-(4-(pyridin-3-yl)-1H- pyrazol-1-yl)pyrido[3,4- d]pyrimidin-4(3H)-one | Scheme A | ¹H NMR (400 MHz, DMSO) δ 8.53 (s, 1H), 8.10 (s, 1H), 7.80 (d, J = 5.09 Hz, 1H), 7.64 (d, J = 3.52 Hz, 1H), 7.54 (s, 1H), 7.47 (s, 1H), 7.32 (d, J = 5.09 Hz, 2H), 6.69 (m, 1H) | 290 |
| 13 | 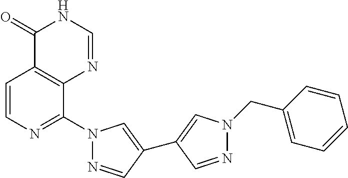 [SMILES: O=c1[nH]cnc2c(-n3cc(-c4cnn(Cc5ccccc5)c4)cn3)nccc12] | 8-(1′-benzyl-1H,1′H-[4,4′- bipyrazol]-1- yl)pyrido[3,4-d]pyrimidin- 4(3H)-one | Scheme A | ¹H NMR (400 MHz, DMSO) δ 9.03 (s, 1H), 8.57 (d, J = 5.09 Hz, 1H), 8.06 (s, 1H), 8.01 (s, 1H), 7.82 (s, 1H), 7.67-7.63 (m, 3H), 7.63-7.60 (m, 2H), 7.55-7.28 (m, 2H), 5.49 (s, 2H) | 369 |
| 14 | 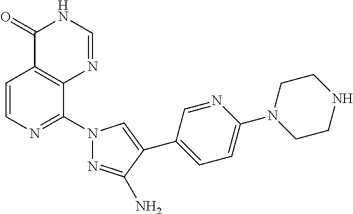 [SMILES: Nc1nn(-c2nccc3c(=O)[nH]cnc23)cc1-c1ccc(N2CCNCC2)nc1] | 8-(3-amino-4-(6- (piperazin-1-yl)pyridin-3- yl)-1H-pyrazol-1- yl)pyrido[3,4-d]pyrimidin- 4(3H)-one | Scheme B | none | 390 |
| 15 | 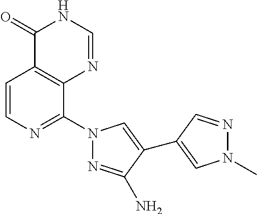 [SMILES: Cn1cc(-c2cn(-c3nccc4c(=O)[nH]cnc34)nc2N)cn1] | 8-(3-amino-1′-methyl- 1H,1′H-[4,4'-bipyrazol]-1- yl)pyrido[3,4-d]pyrimidin- 4(3H)-one | Scheme B | ¹H NMR (400 MHz, DMSO) δ9.02 (s, 1H), 8.45 (m, 1H), 8.28 (s, 1H), 7.87 (s, 1H), 7.85 (d, 1H), 7.72 (s, 1H), 3.93 (s, 3H) | 309 |
| 16 | 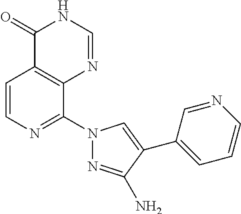 [SMILES: Nc1nn(-c2nccc3c(=O)[nH]cnc23)cc1-c1cccnc1] | 8-(3-amino-4-(pyridin-3- yl)-1H-pyrazol-1- yl)pyrido[3,4-d]pyrimidin- 4(3H)-one | Scheme B | ¹H NMR (400 MHz, DMSO) δ 8.77 (s, 1H), 8.39 (m, 2H), 8.27 (s, 1H), 7.77 (d, 1H), 7.70 (s, 1H), 7.40 (s, 1H), 6.19 (s, 1H) | 306 |
| 17 | 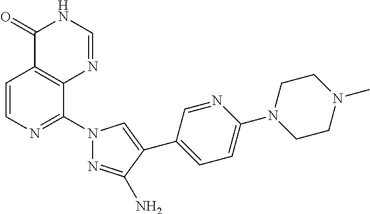 [SMILES: CN1CCN(c2ccc(-c3cn(-c4nccc5c(=O)[nH]cnc45)nc3N)cn2)CC1] | 8-(3-amino-4-(4-(4- methylpiperazin-1- yl)phenyl)-1H-pyrazol-1- yl)pyrido[3,4-d]pyrimidin- 4(3H)-one | Scheme B | ¹H NMR (600 MHz, CD₃OD) δ 9.05 (s, 1H), 8.41-8.47 (m, 1H) 8.34 (s, 1H), 7.85 (br d, J = 4.70 Hz, 1H), 7.44 (d, J = 8.80 Hz, 2H), 7.03 (d, J = 8.80 Hz, 2H), 3.87 (br d, J = 13.50 Hz, 2H), 3.58-3.69 (m, 2H), 3.24-3.28 (m, 2H), 3.08 (br t, J = 12.03 Hz, 2H), 2.93- 2.99 (m, 3H) | 403 |
| 18 | 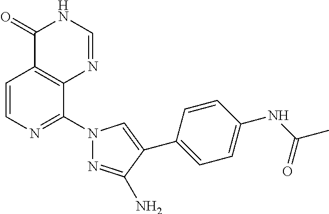 [SMILES: CC(=O)Nc1ccc(-c2cn(-c3nccc4c(=O)[nH]cnc34)nc2N)cc1] | N-(4-(3-amino-1-(4-oxo- 3,4-dihydropyrido[3,4- d]pyrimidin-8-yl)-1H- pyrazol-4- yl)phenyl)acetamide | Scheme B | ¹H NMR (600 MHz, CD₃OD) δ 9.26 (s, 1H), 8.49 (br d, J = 5.28 Hz, 1H), 8.40 (s, 1H), 7.96 (br d, J = 5.28 Hz, 1H). 7.68 (d, J = 8.22 Hz, 2H), 7.54 (d, J = 8.22 Hz, 2H), 2.14 (s, 3H) | 362 |
| 19 | 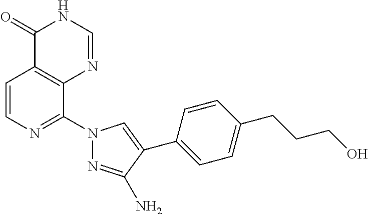 [SMILES: Nc1nn(-c2nccc3c(=O)[nH]cnc23)cc1-c1ccc(CCCO)cc1] | 8-(3-amino-4-(4-(3- hydroxypropyl)phenyl)- 1H-pyrazol-1- yl)pyrido[3,4-d]pyrimidin- 4(3H)-one | Scheme B | ¹H NMR (600 MHz, CD₃OD) δ 9.23 (s, 1H), 8.48 (d, J = 4.69 Hz, 1H), 8.38 (s, 1H), 7.94 (d, J = 5.28 Hz, 1H), 7.42 (s, 1H), 7.39 (br d, 2H), 7.24 (br t, J = 3.52 Hz, 1H), 3.59 (t, J = 6.46 Hz, 2H), 2.75 (t, 2H), 1.85-1.91 (m, 2H) | 363 |
| 20 | 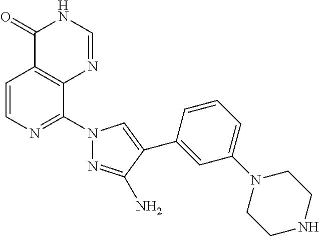 [SMILES: Nc1nn(-c2nccc3c(=O)[nH]cnc23)cc1-c1cccc(N2CCNCC2)c1] | 8-(3-amino-4-(3- (piperazin-1-yl)phenyl)- 1H-pyrazol-1- yl)pyrido[3,4-d]pyrimidin- 4(3H)-one | Scheme B | ¹H NMR (600 MHz, CD₃OD) δ 9.18 (s, 1H), 8.48 (d, J = 5.28 Hz, 1H), 8.41 (s, 1H), 7.93 (d, J = 5.28 Hz, 1H), 7.39 (d, J = 7.92 Hz, 1H), 7.19 (s, 1H), 7.13 (d, J = 7.63 Hz, 1H), 6.98-7.10 (m, 1H), 3.46-3.52 (m, 4H), 3.34-3.45 (m, 4H) | 389 |
| 21 | 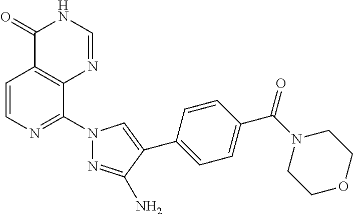 [SMILES: Nc1nn(-c2nccc3c(=O)[nH]cnc23)cc1-c1ccc(C(=O)N2CCOCC2)cc1] | 8-(3-amino-4-(4- (morpholine-4- carbonyl)phenyl)-1H- pyrazol-1-yl)pyrido[3,4- d]pyrimidin-4(3H)-one | Scheme B | ¹H NMR (600 MHz, CD₃OD) δ 9.28 (s, 1H), 8.51 (d, J = 4.70 Hz, 2H), 8.40 (s, 1H), 7.96 (d, J = 5.28 Hz, 1H), 7.71 (d, J = 8.22 Hz, 2H), 7.53 (d, J = 8.22 Hz, 1H), 3.70-3.88 (m, 2H), 3.57-3.69 (m, 2H) | 418 |
| 22 | 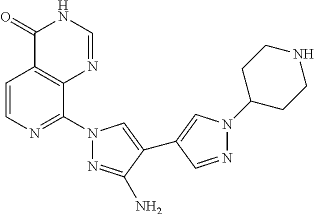 [SMILES: Nc1nn(-c2nccc3c(=O)[nH]cnc23)cc1-c1cnn(C2CCNCC2)c1] | 8-(3-amino-1′-(piperidin- 4-yl)-1H,1′H-[4,4′- bipyrazol]-1- yl)pyrido[3,4-d]pyrimidin- 4(3H)-one | Scheme B | ¹H NMR (600 MHz, CD₃OD) δ 9.09 (s, 1H), 8.46 (d. J = 5.28 Hz, 1H), 8.33 (s, 1H), 8.01 (s, 1H), 7.89 (d, J = 4.70 Hz, 1H), 7.83 (s, 1H), 4.50- 4.67 (m, 1H), 3.58 (br d, J = 13.50 Hz, 2H), 3.17-3.26 (m, 2H), 2.23-2.39 (m, 4H) | 378 |
| 23 | 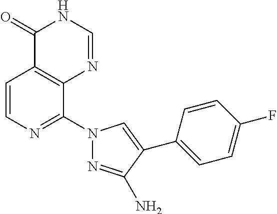 [SMILES: Nc1nn(-c2nccc3c(=O)[nH]cnc23)cc1-c1ccc(F)cc1] | 8-(3-amino-4-(4- fluorophenyl)-1H-pyrazol- 1-yl)pyrido[3,4- d]pyrimidin-4(3H)-one | Scheme B | ¹H NMR (600 MHz, CD₃OD) δ 9.24 (s, 1H), 8.49 (d, J = 4.70 Hz, 1H), 8.38 (s, 1H), 7.91 (d, J = 5.28 Hz, 1H), 7.59 (d, 2H), 7.24 (m, 2H) | 323 |
| 24 | 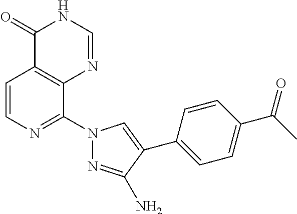 [SMILES: CC(=O)c1ccc(-c2cn(-c3nccc4c(=O)[nH]cnc34)nc2N)cc1] | 8-(4-(4-acetylphenyl)-3- amino-1H-pyrazol-1- yl)pyrido[3,4-d]pyrimidin- 4(3H)-one | Scheme B | ¹H NMR (600 MHz, CD₃OD) δ 8.75 (s, 1H), 8.49 (d, J = 5.28 Hz, 1H), 8.39 (s, 1H), 7.98 (d, 1H), 7.75-7.78 (m, 2H), 7.57-7.60 (m, 2H), 2.73 (d, 3H) | 347 |
| 25 | 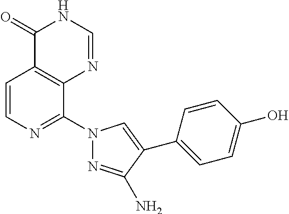 [SMILES: Nc1nn(-c2nccc3c(=O)[nH]cnc23)cc1-c1ccc(O)cc1] | 8-(3-amino-4-(4- hydroxyphenyl)-1H- pyrazol-1-yl)pyrido[3,4- d]pyrimidin-4(3H)-one | Scheme B | ¹H NMR (600 MHz, CD₃OD) δ 9.06 (s, 1H), 8.46 (d, 1H), 8.32 (s, 1H), 7.89 (d, J = 5.28 Hz, 1H), 7.39 (d, J = 8.8 Hz, 2H), 6.88 (d, J = 8.8 Hz, 2H), | 321 |
| 26 | 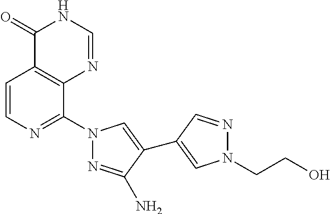 [SMILES: Nc1nn(-c2nccc3c(=O)[nH]cnc23)cc1-c1cnn(CCO)c1] | 8-(3-amino-1′-(2- hydroxyethyl)-1H,1′H- [4,4′-bipyrazol]-1- yl)pyrido[3,4-d]pyrimidin- 4(3H)-one | Scheme B | ¹H NMR (400 MHz, CD₃OD) δ 9.26 (d, 1H), 8.47 (d, 1H), 8.42 (d, 1H), 8.04-8.00 (d, 1H), 7.95 (d, 1H), 7.84-7.81 (s, 1H), 4.78 (t, 1H), 4.58 (t, 1H), 4.28 (t, 1H), 3.92 (t, 1H) | 338 |
| 27 | 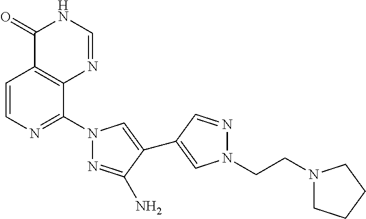 [SMILES: Nc1nn(-c2nccc3c(=O)[nH]cnc23)cc1-c1cnn(CCN2CCCC2)c1] | 8-(3-amino-1′-(2- (pyrrolidin-1-yl)ethyl)- 1H,1′H-[4,4′-bipyrazol]-1- yl)pyrido[3,4-d]pyrimidin- 4(3H)-one | Scheme B | ¹H NMR (400 MHz, CD₃OD) δ 9.04 (s, 1H), 8.45 (d, 1H), 8.41 (s, 1H), 8.04 (s, 1H), 7.87 (d, 1H), 7.83 (s, 1H), 4.61 (t, 2H), 3.78 (t, 2H), 3.15 (br s, 2H), 2.16 (br s, 2H) | 391 |
| 28 | 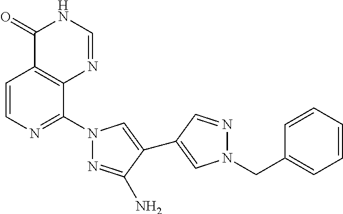 [SMILES: Nc1nn(-c2nccc3c(=O)[nH]cnc23)cc1-c1cnn(Cc2ccccc2)c1] | 8-(3-amino-1′-benzyl- 1H,1′H-[4,4′-bipyrazol]-1- yl)pyrido[3,4-d]pyrimidin- 4(3H)-one | Scheme B | ¹H NMR (400 MHz, CD₃OD) δ 9.22 (s, 1H), 8.46 (d, 1H), 8.38 (s, 1H), 8.05 (s, 1H), 7.93 (d, 1H), 7.83 (s, 1H), 7.40-7.24 (m, 5H), 5.39 (s, 2H) | 384 |
| 29 | 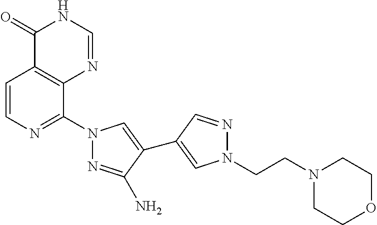 [SMILES: Nc1nn(-c2nccc3c(=O)[nH]cnc23)cc1-c1cnn(CCN2CCOCC2)c1] | 8-(3-amino-1′-(2- morpholinoethyl)-1H,1′H- [4,4′-bipyrazol]-1- yl)pyrido[3,4-d]pyrimidin- 4(3H)-one | Scheme B | ¹H NMR (400 MHz, CD₃OD) δ 9.02 (s, 1H), 8.46 (d, 1H), 8.40 (s, 1H), 8.03 (s, 1H), 7.88 (d, 1H), 7.83 (s, 1H), 4.67 (t, 2H), 3.76 (t, 2H) | 407 |
| 30 | 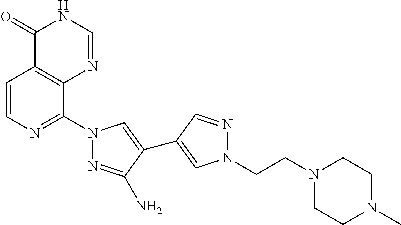 [SMILES: CN1CCN(CCn2cc(-c3cn(-c4nccc5c(=O)[nH]cnc45)nc3N)cn2)CC1] | 8-(3-amino-1′-(2-(4- methylpiperazin-1- yl)ethyl)-1H,1′H-[4,4′- bipyrazol]-1- yl)pyrido[3,4-d]pyrimidin- 4(3H)-one | Scheme B | ¹H NMR (400 MHz, CD₃OD) δ 9.16 (s, 1H), 8.47 (d, J = 5.09 Hz, 1H), 8.41 (s, 1H), 8.02 (1H), 7.93 (d, J = 5.48 Hz, 1H), 7.81 (s, 1H), 4.35 (t, 2H), 2.99 (t, 2H), 2.86 (s, 3H) | 420 |
| 31 | 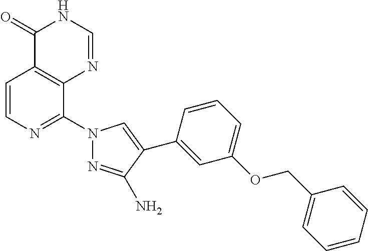 [SMILES: Nc1nn(-c2nccc3c(=O)[nH]cnc23)cc1-c1cccc(OCc2ccccc2)c1] | 8-(3-amino-4-(3- (benzyloxy)phenyl)-1H- pyrazol-1-yl)pyrido[3,4- d]pyrimidin-4(3H)-one | Scheme B | ¹H NMR (400 MHz, CD₃OD) δ 9.18 (s, 1H), 8.54 (s, 1H), 8.46 (d, 1H), 8.36 (s, 1H), 7.94 (d, 2H), 7.22 (d, 2H), 7.12 (m, 3H), 6.98 (m, 2H), 4.95 (s, 2H) | 410 |
| 32 | 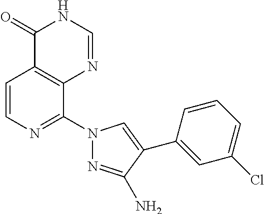 [SMILES: Nc1nn(-c2nccc3c(=O)[nH]cnc23)cc1-c1cccc(Cl)c1] | 8-(3-amino-4-(3- chlorophenyl)-1H-pyrazol- 1-yl)pyrido[3,4- d]pyrimidin-4(3H)-one | Scheme B | ¹H NMR (400 MHz, CD₃OD) δ 9.16 (s, 1H), 8.57 (d, 1H), 8.44 (s, 1H), 8.11 (d, 1H), 7.53 (d, 2H), 7.42 (d, 2H) | 339 |
| 33 | 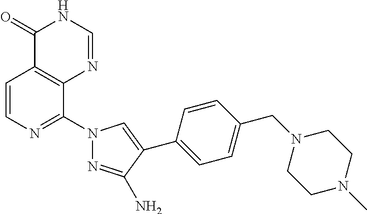 [SMILES: CN1CCN(Cc2ccc(-c3cn(-c4nccc5c(=O)[nH]cnc45)nc3N)cc2)CC1] | 8-(3-amino-4-(4-((4- methylpiperazin-1- yl)methyl)phenyl)-1H- pyrazol-1-yl)pyrido[3,4- d]pyrimidin-4(3H)-one | Scheme B | ¹H NMR (400 MHz, CD₃OD) δ 9.23 (s, 1H), 8.51 (d, J = 5.09 Hz, 1H), 8.42 (s, 1H), 7.95 (d, J = 5.09 Hz, 1H), 7.66 (d, J = 8.22 Hz, 2H), 7.55 (d, J = 8.22 Hz, 2H), 4.07 (s, 2H), 3.46 (br s, 4H), 3.19 (br s, 4H), 2.93 (s, 3H) | 416 |
| 34 | 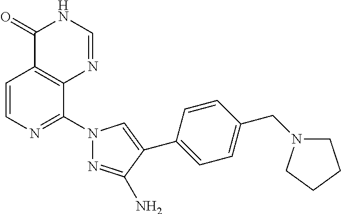 [SMILES: Nc1nn(-c2nccc3c(=O)[nH]cnc23)cc1-c1ccc(CN2CCCC2)cc1] | 8-(3-amino-4-(4- (pyrrolidin-1- ylmethyl)phenyl)-1H- pyrazol-1-yl)pyrido[3,4- d]pyrimidin-4(3H)-one | Scheme B | ¹H NMR (400 MHz, CD₃OD) δ 8.35 (s, 1H), 7.71 (d, 1H), 7.48 (s, 1H), 7.12 (d, 1H), 6.97 (d, 1H), 6.93 (d, 1H), 6.82 (d, 1H), 6.78 (d, 1H), 3.62 (d, 2H) | 387 |
| 35 | 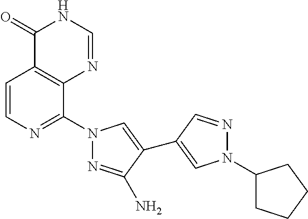 [SMILES: Nc1nn(-c2nccc3c(=O)[nH]cnc23)cc1-c1cnn(C2CCCC2)c1] | 8-(3-amino-1′-cyclopentyl- 1H,1′H-[4,4′-bipyrazol]-1- yl)pyrido[3,4-d]pyrimidin- 4(3H)-one | Scheme B | ¹H NMR (400 MHz, CD₃OD) δ 9.24 (s, 1H), 8.48 (d, J = 5.48 Hz, 1H), 8.41 (s, 1H), 8.04 (s, 1H), 7.95 (d, J = 5.48 Hz, 1H), 7.80 (s, 1H), 4.76 (m, 1H), 2.21 (m, 2H), 2.05 (m, 2H), 1.92 (m, 2H), 1.76 (m, 2H) | 362 |
| 36 | 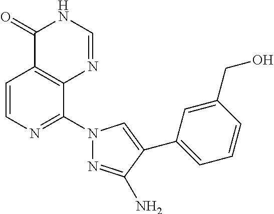 [SMILES: Nc1nn(-c2nccc3c(=O)[nH]cnc23)cc1-c1cccc(CO)c1] | 8-(3-amino-4-(3- (hydroxymethyl)phenyl)- 1H-pyrazol-1- yl)pyrido[3,4-d]pyrimidin- 4(3H)-one | Scheme B | none | 334 |
| 37 | 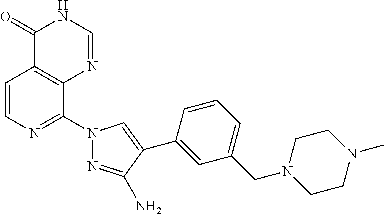 [SMILES: CN1CCN(Cc2cccc(-c3cn(-c4nccc5c(=O)[nH]cnc45)nc3N)c2)CC1] | 8-(3-amino-4-(3-((4- methylpiperazin-1- yl)methyl)phenyl)-1H- pyrazol-1-yl)pyrido[3,4- d]pyrimidin-4(3H)-one | Scheme B | none | 416 |
| 38 | 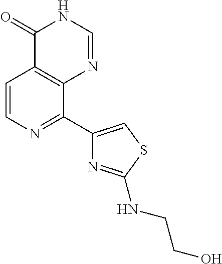 [SMILES: O=c1[nH]cnc2c(-c3csc(NCCO)n3)nccc12] | 8-(2-((2- hydroxyethyl)amino) thiazol-4-yl)pyrido[3,4- d]pyrimidin-4(3H)-one | Scheme B | ¹H NMR (400 MHz, CD₃OD) δ 8.69 (d, J = 5.09 Hz, 1H), 8.39 (s, 1H), 8.28 (s, 1H), 8.11 (d, J = 5.09 Hz, 1H), 3.81-3.86 (m, 2H), 3.60-3.64 (m, 2H) | 290 |
| 39 | 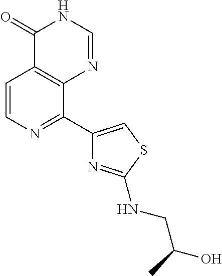 [SMILES: C[C@H](O)CNc1nc(-c2nccc3c(=O)[nH]cnc23)cs1] | (S)-8-(2-((2- hydroxypropyl)amino) thiazol-4-yl)pyrido[3,4- d]pyrimidin-4(3H)-one | Scheme B | ¹H NMR (400 MHz, CD₃OD) δ 8.69 (d, J = 5.09 Hz, 1H), 8.38 (s, 1H), 8.28 (s, 1H), 8.11 (d, J = 5.09 Hz, 1H), 3.55-3.65 (m, 2H), 3.38-3.51 (m, 1H), 1.27 (d, J = 6.26 Hz, 3H) | 304 |
| 40 | 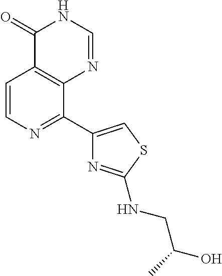 [SMILES: C[C@@H](O)CNc1nc(-c2nccc3c(=O)[nH]cnc23)cs1] | (R)-8-(2-((2- hydroxypropyl)amino) thiazol-4-yl)pyrido[3,4- d]pyrimidin-4(3H)-one | Scheme B | ¹H NMR (400 MHz, CD₃OD) δ 8.69 (d, J = 5.09 Hz, 1H), 8.38 (s, 1H), 8.28 (s, 1H), 8.11 (d, J = 5.09 Hz, 1H), 3.53-3.65 (m, 2H), 3.41 (br dd, J = 14.09, 7.04 Hz, 1H), 1.27 (d, J = 6.26 Hz, 3H) | 304 |
| 41 | 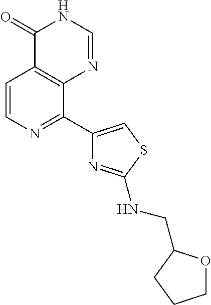 [SMILES: O=c1[nH]cnc2c(-c3csc(NCC4CCCO4)n3)nccc12] | 8-(2-(((tetrahydrofuran-2- yl)methyl)amino)thiazol- 4-yl)pyrido[3,4- d]pyrimidin-4(3H)-one | Scheme B | ¹H NMR (400 MHz, CD₃OD) δ 8.70 (d, J = 5.09 Hz, 1H), 8.38 (s, 1H), 8.28 (s, 1H), 8.11 (d, J = 5.48 Hz, 1H), 3.65-3.90 (m, 3H), 3.38-3.60 (m, 2H), 1.95-2.05 (m, 2H), 1.80-1.90 (m, 2H) | 330 |
| 42 | 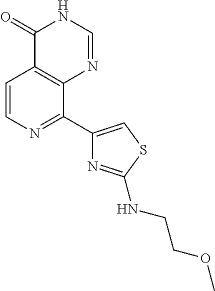 [SMILES: COCCNc1nc(-c2nccc3c(=O)[nH]cnc23)cs1] | 8-(2-((2- methoxyethyl)amino) thiazol-4-yl)pyrido[3,4- d]pyrimidin-4(3H)-one | Scheme B | ¹H NMR (400 MHz, CD₃OD) δ 8.70 (d, J = 5.09 Hz, 1H), 8.40 (s, 1H), 8.29 (s, 1H), 8.11 (d, J = 5.48 Hz, 1H), 3.70 (s, 4H), 3.45 (s, 3H) | 304 |
| 43 | 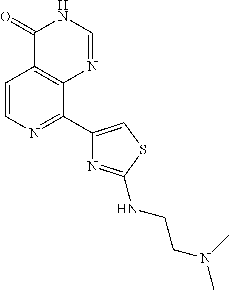 [SMILES: CN(C)CCNc1nc(-c2nccc3c(=O)[nH]cnc23)cs1] | 8-(2-((2- (dimethylamino)ethyl) amino)thiazol-4- yl)pyrido[3,4-d]pyrimidin- 4(3H)-one | Scheme B | ¹H NMR (400 MHz, CD₃OD) δ 8.70 (d, J = 5.09 Hz, 1H), 8.63 (s, 1H), 8.29 (s, 1H), 8.09 (d, J = 5.48 Hz, 1H), 3.78-3.84 (m, 2H), 3.40-3.48 (m, 2H), 3.00 (s, 6H) | 317 |
| 44 | 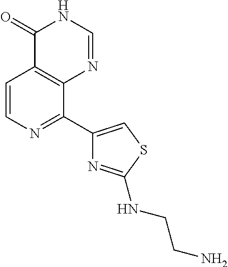 [SMILES: NCCNc1nc(-c2nccc3c(=O)[nH]cnc23)cs1] | 8-(2-((2- aminoethyl)amino)thiazol- 4-yl)pyrido[3,4- d]pyrimidin-4(3H)-one | Scheme B | ¹H NMR (400 MHz, CD₃OD) δ 8.69 (d, J = 5.87 Hz, 1H), 8.60 (s, 1H), 8.32 (s, 1H), 8.16 (d, J = 5.48 Hz, 1H), 3.74-3.82 (m, 2H), 3.25-3.30 (m, 2H) | 289 |
| 45 | 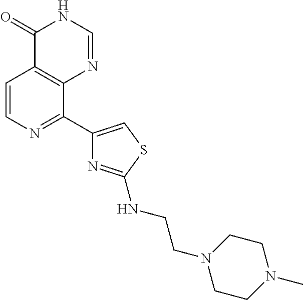 [SMILES: CN1CCN(CCNc2nc(-c3nccc4c(=O)[nH]cnc34)cs2)CC1] | 8-(2-((2-(4- methylpiperazin-1- yl)ethyl)amino)thiazol-4- yl)pyrido[3,4-d]pyrimidin- 4(3H)-one | Scheme B | ¹H NMR (400 MHz, CD₃OD) δ 8.72 (d, J = 5.09 Hz, 1H), 8.51 (s, 1H), 8.30 (s, 1H), 8.14 (d, J = 5.09 Hz, 1H), 3.68 (t, J = 5.67 Hz, 2H), 3.29 (dt, J = 3.13, 1.57 Hz, 8H), 2.95-3.07 (m, 2H), 2.92 (s, 3H) | 372 |
| 46 | 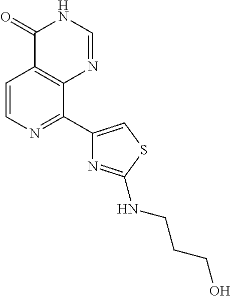 [SMILES: O=c1[nH]cnc2c(-c3csc(NCCCO)n3)nccc12] | 8-(2-((3- hydroxypropyl)amino) thiazol-4-yl)pyrido[3,4- d]pyrimidin-4(3H)-one | Scheme B | ¹H NMR (400 MHz, CD₃OD) δ 8.7 (d, J = 4.7 Hz, 1H), 8.51 (s, 1H), 8.29 (s, 1H), 8.15 (d, J = 5.09 Hz, 1H), 3.72 (t, J = 5.87 Hz, 2H), 3.63 (m, 2H), 1.96 (quin, J = 6.26 Hz, 2H) | 304 |
| 47 | 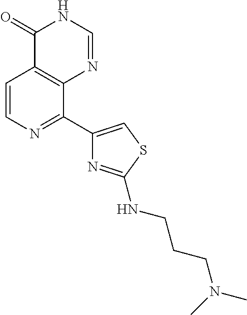 [SMILES: CN(C)CCCNc1nc(-c2nccc3c(=O)[nH]cnc23)cs1] | 8-(2-((3- (dimethylamino)propyl) amino)thiazol-4- yl)pyrido[3,4-d]pyrimidin- 4(3H)-one | Scheme B | ¹H NMR (400 MHz, CD₃OD) δ 8.70 (d, J = 5.48 Hz, 1H), 8.55 (s, 1H), 8.31 (s, 1H), 8.14 (d, J = 5.48 Hz, 1H), 3.62 (t, J = 6.65 Hz, 2H), 3.26 (br d, J = 7.83 Hz, 2H), 2.91 (s, 6H), 2.10-2.25 (m, 2H) | 331 |
| 48 | 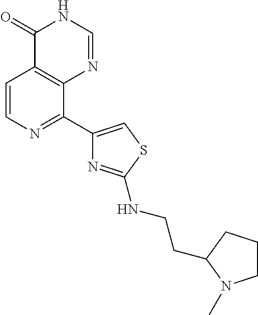 [SMILES: CN1CCCC1CCNc1nc(-c2nccc3c(=O)[nH]cnc23)cs1] | 8-(2-((2-(1- methylpyrrolidin-2- yl)ethyl)amino)thiazol-4- yl)pyrido[3,4-d]pyrimidin- 4(3H)-one | Scheme B | none | 357 |
| 49 | 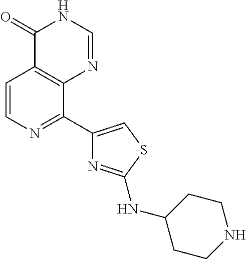 [SMILES: O=c1[nH]cnc2c(-c3csc(NC4CCNCC4)n3)nccc12] | 8-(2-(piperidin-4- ylamino)thiazol-4- yl)pyrido[3,4-d]pyrimidin- 4(3H)-one | Scheme B | ¹H NMR (400 MHz, CD₃OD) δ 8.66 (d, J = 5.87 Hz, 1H), 8.63 (s, 1H), 8.35 (s, 1H), 8.20 (d, J = 5.48 Hz, 1H), 3.45-3.55 (m, 2H), 3.29 (m, 1H), 3.13-3.26 (m, 2H), 2.37 (br dd, J = 14.28, 3.33 Hz, 2H), 1.83-1.99 (m, 2H) | 329 |
| 50 | 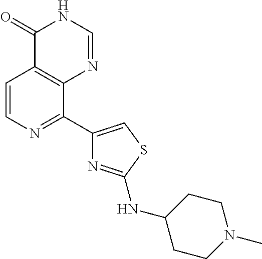 [SMILES: CN1CCC(Nc2nc(-c3nccc4c(=O)[nH]cnc34)cs2)CC1] | 8-(2-((1-methylpiperidin- 4-yl)amino)thiazol-4- yl)pyrido[3,4-d]pyrimidin- 4(3H)-one | Scheme B | none | 343 |
| 51 | 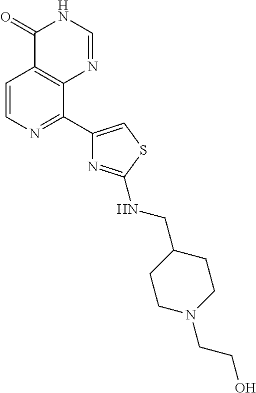 [SMILES: O=c1[nH]cnc2c(-c3csc(NCC4CCN(CCO)CC4)n3)nccc12] | 8-(2-(((1-(2- hydroxyethyl)piperidin-4- yl)methyl)amino)thiazol- 4-yl)pyrido[3,4- d]pyrimidin-4(3H)-one | Scheme B | ¹H NMR (400 MHz, CD₃OD) δ 8.69 (d, J = 5.48 Hz, 1H), 8.50 (s, 1H), 8.31 (s, 1H), 8.16 (d, J = 5.09 Hz, 1H), 4.00 (s, 1H), 3.87 (m, 2H), 3.71 (br d, 1H), 3.48 (d, 2H), 3.24 (m, 2H), 3.04 (br t, 3H), 2.14 (br d, 2H), 1.64 (br d, 2H) | 386 |
| 52 | 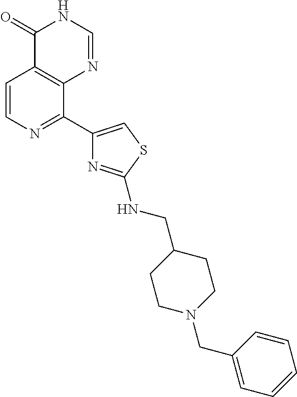 [SMILES: O=c1[nH]cnc2c(-c3csc(NCC4CCN(Cc5ccccc5)CC4)n3)nccc12] | 8-(2-(((1-benzylpiperidin- 4- yl)methyl)amino)thiazol- 4-yl)pyrido[3,4- d]pyrimidin-4(3H)-one | Scheme B | none | 433 |
| 53 | 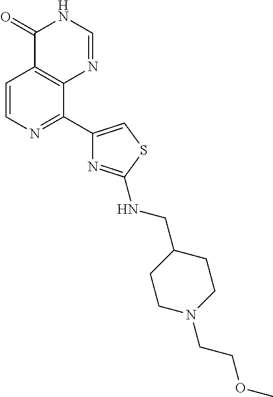 [SMILES: COCCN1CCC(CNc2nc(-c3nccc4c(=O)[nH]cnc34)cs2)CC1] | 8-(2-(((1-(2- methoxyethyl)piperidin-4- yl)methyl)amino)thiazol- 4-yl)pyrido[3,4- d]pyrimidin-4(3H)-one | Scheme B | none | 401 |
| 54 | 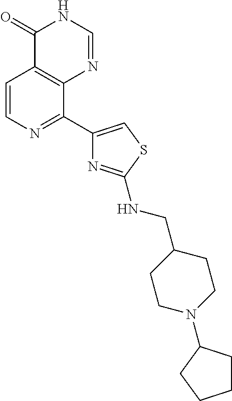 [SMILES: O=c1[nH]cnc2c(-c3csc(NCC4CCN(C5CCCC5)CC4)n3)nccc12] | 8-(2-(((1- cyclopentylpiperidin-4- yl)methyl)amino)thiazol- 4-yl)pyrido[3,4- d]pyrimidin-4(3H)-one | Scheme B | none | 411 |
| 55 | 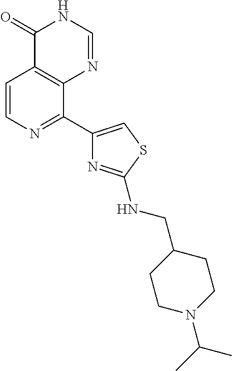 [SMILES: CC(C)N1CCC(CNc2nc(-c3nccc4c(=O)[nH]cnc34)cs2)CC1] | 8-(2-(((1- isopropylpiperidin-4- yl)methyl)amino)thiazol- 4-yl)pyrido[3,4- d]pyrimidin-4(3H)-one | Scheme B | none | 385 |
| 56 | 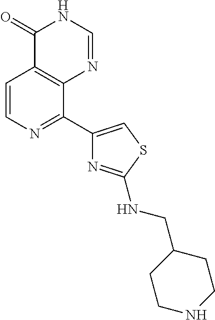 [SMILES: O=c1[nH]cnc2c(-c3csc(NCC4CCNCC4)n3)nccc12] | 8-(2-((piperidin-4- ylmethyl)amino)thiazol-4- yl)pyrido[3,4-d]pyrimidin- 4(3H)-one | Scheme B | none | 342 |
| 57 | 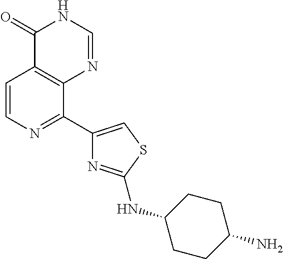 [SMILES: N[C@H]1CC[C@@H](Nc2nc(-c3nccc4c(=O)[nH]cnc34)cs2)CC1] | 8-(2-(((1s,4s)-4- aminocyclohexyl)amino) thiazol-4-yl)pyrido[3,4- d]pyrimidin-4(3H)-one | Scheme B | none | 342 |
| 58 | 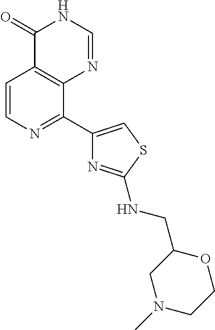 [SMILES: CN1CCOC(CNc2nc(-c3nccc4c(=O)[nH]cnc34)cs2)C1] | 8-(2-(((4- methylmorpholin-2- yl)methyl)amino)thiazol- 4-yl)pyrido[3,4- d]pyrimidin-4(3H)-one | Scheme B | ¹H NMR (400 MHz, CD₃OD) δ 8.68 (d, J = 5.48 Hz, 1H), 8.44 (s, 1H), 8.32 (s, 1H), 8.12 (d, J = 5.48 Hz, 1H), 4.23 (d, 1H), 4.07 (m, 1H), 3.83 (m, 3H), 3.70-3.38 (m, 4 H), 2.94 (s, 3H) | 358 |
| 59 | 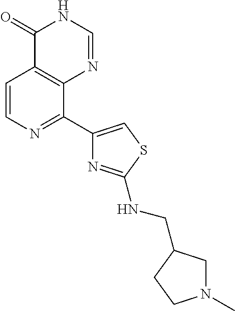 [SMILES: CN1CCC(CNc2nc(-c3nccc4c(=O)[nH]cnc34)cs2)C1] | 8-(2-(((1- methylpyrrolidin-3- yl)methyl)amino)thiazol- 4-yl)pyrido[3,4- d]pyrimidin-4(3H)-one | Scheme B | ¹H NMR (400 MHz, CD₃OD) δ 8.71 (d, 1H), 8.42 (s, 1H), 8.27 (s, 1H), 8.07 (d, 1H), 2.96 (s, 3H), 2.76 (s, 2H), 2.40 (m, 3H), 2.06-1.87 (m, 2H), 1.5 (m, 2H) | 342 |
| 60 | 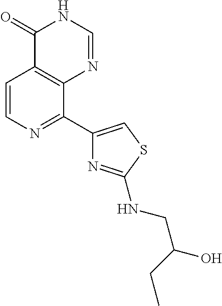 [SMILES: CCC(O)CNc1nc(-c2nccc3c(=O)[nH]cnc23)cs1] | 8-(2-((2- hydroxybutyl)amino) thiazol-4-yl)pyrido[3,4- d]pyrimidin-4(3H)-one | Scheme B | none | 317 |
| 61 | 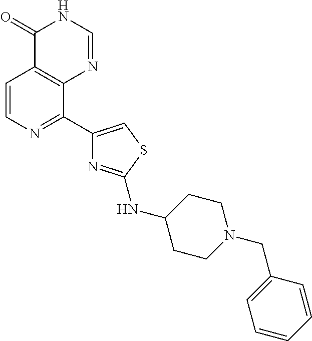 [SMILES: O=c1[nH]cnc2c(-c3csc(NC4CCN(Cc5ccccc5)CC4)n3)nccc12] | 8-(2-((1-benzylpiperidin- 4-yl)amino)thiazol-4- yl)pyrido[3,4-d]pyrimidin- 4(3H)-one | Scheme B | ¹H NMR (400 MHz, CD₃OD) δ 8.67 (d, J = 5.48 Hz, 1H), 8.62 (s, 1H), 8.35 (s, 1H), 8.22 (d, J = 5.48 Hz, 1H), 7.6-7.46 (m, 5H), 4.38 (s, 2H), 4.05 (m, 1H), 3.61 (br d, 2H), 3.20 (br t, 2H), 2.43 (br d, 2H), 1.91 (br t, 2H) | 257 |
| 62 | 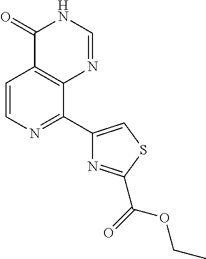 [SMILES: CCOC(=O)c1nc(-c2nccc3c(=O)[nH]cnc23)cs1] | ethyl 4-(4-oxo-3,4- dihydropyrido[3,4- d]pyrimidin-8-yl)thiazole- 2-carboxylate | Scheme B | ¹H NMR (400 MHz, CD₃OD) δ 9.30 (s, 1H), 8.73 (d, J = 5.48 Hz, 1H), 8.36 (s, 1H), 8.25 (d, J = 5.87 Hz, 1H), 4.51 (q, 2H), 1.45 (t, 3H) | 419 |
| 63 | 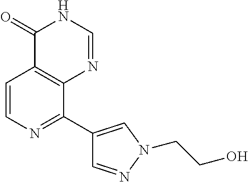 [SMILES: O=c1[nH]cnc2c(-c3cnn(CCO)c3)nccc12] | 8-(1-(2-hydroxyethyl)-1H- pyrazol-4-yl)pyrido[3,4- d]pyrimidin-4(3H)-one | Scheme B | ¹H NMR (400 MHz, CD₃OD) δ 8.95 (s, 1H), 8.50 (m, 2H), 8.31 (s, 1H), 8.09 (d, J = 5.48 Hz, 1H), 4.34 (t, J = 5.09, 5.09 Hz, 2H), 3.94 (t, J = 5.28 Hz, 5.28 Hz, 2H) | 302 |
| 64 | 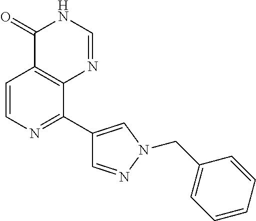 [SMILES: O=c1[nH]cnc2c(-c3cnn(Cc4ccccc4)c3)nccc12] | 8-(1-benzyl-1H-pyrazol-4- yl)pyrido[3,4-d]pyrimidin- 4(3H)-one | Scheme B | ¹H NMR (400 MHz, CD₃OD) δ 8.68 (d, 1H), 8.09 (s, 1H), 8.05 (d, 1H), 7.64 (s, 1H), 7.32 (m, 2H), 7.09 (m, 2H), 6.94 (s, 1H), 6.91 (m, 1H), 5.84 (s, 2H) | 303 |
| 65 | 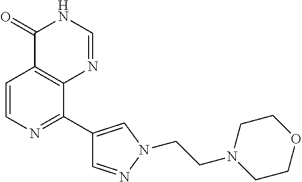 [SMILES: O=c1[nH]cnc2c(-c3cnn(CCN4CCOCC4)c3)nccc12] | 8-(1-(2-morpholinoethyl)- 1H-pyrazol-4- yl)pyrido[3,4-d]pyrimidin- 4(3H)-one | Scheme B | ¹H NMR (400 MHz, CD₃OD) δ 8.89 (s, 1H), 8.57 (d, 1H), 8.51 (s, 1H), 8.21 (s, 1H), 7.96 (d, 1H), 4.73 (t, 2H), 3.77 (t, 2H) | 326 |
| 66 | 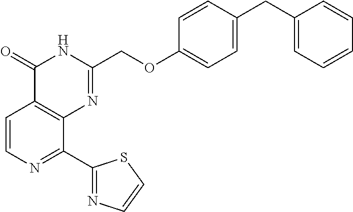 [SMILES: O=c1[nH]c(COc2ccc(Cc3ccccc3)cc2)nc2c(-c3nccs3)nccc12] | 2-((4- benzylphenoxy)methyl)-8- (thiazol-2-yl)pyrido[3,4- d]pyrimidin-4(3H)-one | Scheme C | ¹H NMR (400 MHz, DMSO) δ 8.72 (s, 1H), 8.01 (m, 2H), 7.85 (s, 1H), 7.70-7.49 (m, 5H), 7.30-7.10 (m, 4H), 3.83 (s, 2H), 3.60 (s, 2H) | 427 |
| 67 | 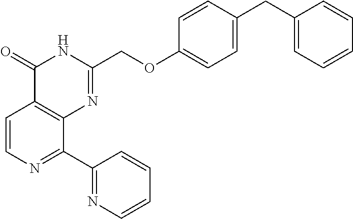 [SMILES: O=c1[nH]c(COc2ccc(Cc3ccccc3)cc2)nc2c(-c3ccccn3)nccc12] | 2-((4- benzylphenoxy)methyl)-8- (pyridin-2-yl)pyrido[3,4- d]pyrimidin-4(3H)-one | Scheme C | ¹H NMR (400 MHz, DMSO) δ 8.79 (m, 1H), 8.73 (s, 1H), 8.43 (m, 1H), 8.10 (m, 1H), 8.05 (t, 2H), 7.68 (t, 1H), 7.31-7.10 (m, 6H), 7.02 (d, 2H) | 421 |
| 68 | 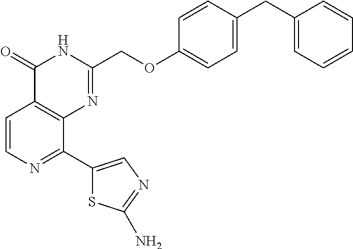 [SMILES: Nc1ncc(-c2nccc3c(=O)[nH]c(COc4ccc(Cc5ccccc5)cc4)nc23)s1] | 8-(2-aminothiazol-5-yl)-2- ((4- benzylphenoxy)methyl) pyrido[3,4-d]pyrimidin- 4(3H)-one | Scheme C | none | 442 |
| 69 | 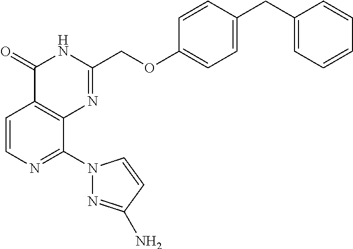 [SMILES: Nc1ccn(-c2nccc3c(=O)[nH]c(COc4ccc(Cc5ccccc5)cc4)nc23)n1] | 8-(3-amino-1H-pyrazol-1- yl)-2-((4- benzylphenoxy)methyl) pyrido[3,4-d]pyrimidin- 4(3H)-one | Scheme C | none | 425 |
| 70 | 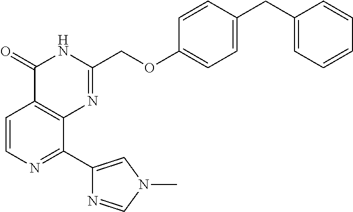 [SMILES: Cn1cnc(-c2nccc3c(=O)[nH]c(COc4ccc(Cc5ccccc5)cc4)nc23)c1] | 2-((4- benzylphenoxy)methyl)-8- (1-methyl-1H-imidazol-4- yl)pyrido[3,4-d]pyrimidin- 4(3H)-one | Scheme C | ¹H NMR (400 MHz, CDCl₃) δ 8.49 (d, J = 3.91 Hz, 1H), 7.82 (d, J = 5.09 Hz, 1H), 7.72 (s, 1H), 7.51 (s, 1H), 7.09-7.31 (m, 7H), 6.93 (d, J = 8.61 Hz, 2H), 5.04 (s, 2H), 3.92 (s, 2H), 3.79 (s, 3H) | 424 |
| 71 | 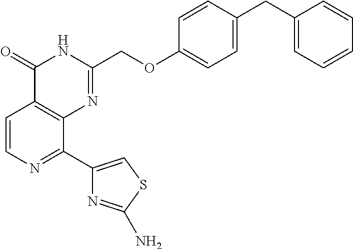 [SMILES: Nc1nc(-c2nccc3c(=O)[nH]c(COc4ccc(Cc5ccccc5)cc4)nc23)cs1] | 8-(2-aminothiazol-4-yl)-2- ((4- benzylphenoxy)methyl) pyrido[3,4-d]pyrimidin- 4(3H)-one | Scheme C | none | 442 |
| 72 | 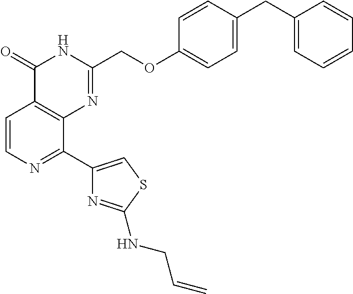 [SMILES: C=CCNc1nc(-c2nccc3c(=O)[nH]c(COc4ccc(Cc5ccccc5)cc4)nc23)cs1] | 8-(2-(allylamino)thiazol-4- yl)-2-((4- benzylphenoxy)methyl) pyrido[3,4-d]pyrimidin- 4(3H)-one | Scheme C | ¹H NMR (400 MHz, DMSO) δ 8.65 (d, J = 5.48 Hz, 1H), 7.97 (d, 1H), 7.93 (s, 1H), 7.61-7.45 (m, 2H), 7.23 (m, 2H), 7.15 (t, 3H), 7.01 (d, 2H), 5.15 (m, 2H), 3.85 (m, 1H), 3.59 (m, 2H) | 482 |
| 73 | 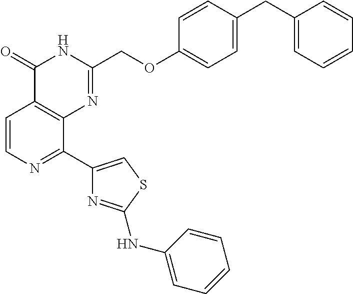 [SMILES: O=c1[nH]c(COc2ccc(Cc3ccccc3)cc2)nc2c(-c3csc(Nc4ccccc4)n3)nccc12] | 2-((4- benzylphenoxy)methyl)-8- (2-(phenylamino)thiazol- 4-yl)pyrido[3,4- d]pyrimidin-4(3H)-one | Scheme C | ¹H NMR (400 MHz, DMSO- d₆) δ 10.28 (br s, 1H), 8.67 (d, J = 5.48 Hz, 1H), 7.91 (d, J = 5.09 Hz, 1H), 7.84-7.89 (m, 1H), 7.69 (br d, J = 7.83 Hz, 2H), 7.05-7.32 (m, 9H), 6.80-7.04 (m, 4H), 5.12 (s, 2H), 3.84 (s, 2H) | 518 |
| 74 | 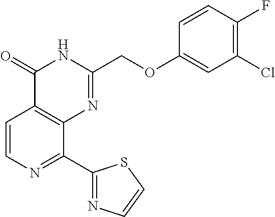 [SMILES: O=c1[nH]c(COc2ccc(F)c(Cl)c2)nc2c(-c3nccs3)nccc12] | 2-((3-chloro-4- fluorophenoxy)methyl)-8- (thiazol-2-yl)pyrido[3,4- d]pyrimidin-4(3H)-one | Scheme C | ¹H NMR (400 MHz, DMSO) δ 8.72 (s, 1H), 8.00 (s, 1H), 7.87 (m, 1H), 7.59 (m, 2H), 7.53 (m, 1H), 7.11 (m, 1H), 5.15 (s, 2H) | 389 |
| 75 | 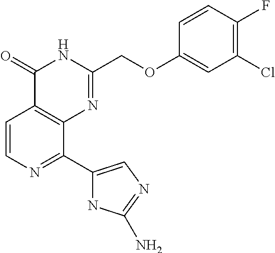 [SMILES: Nc1ncc(-c2nccc3c(=O)[nH]c(COc4ccc(F)c(Cl)c4)nc23)[nH]1] | 8-(2-aminothiazol-5-yl)-2- ((3-chloro-4- fluorophenoxy)methyl) pyrido[3,4-d]pyrimidin- 4(3H)-one | Scheme C | ¹H NMR (400 MHz, DMSO- d₆) δ 12.83 (br s, 1H), 8.42 (d, J = 5.09 Hz, 1H), 8.30 (s, 1H), 7.59 (d, J = 5.09 Hz, 1H), 7.45 (br s, 2H), 7.27- 7.41 (m, 2H), 7.01-7.12 (m, 1H), 5.13 (s, 2H) | 404 |
| 76 | 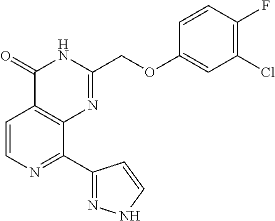 [SMILES: O=c1[nH]c(COc2ccc(F)c(Cl)c2)nc2c(-c3cc[nH]n3)nccc12] | 2-((3-chloro-4- fluorophenoxy)methyl)-8- (1H-pyrazol-3- yl)pyrido[3,4-d]pyrimidin- 4(3H)-one | Scheme C | ¹H NMR (400 MHz, DMSO- d₆) δ 8.65 (d, J = 5.09 Hz, 1H), 7.88 (d, J = 4.70 Hz, 1H), 7.48-7.62 (m, 2H), 7.28- 7.44 (m, 1H), 7.07-7.19 (m, 2H), 6.44-6.59 (m, 1H), 5.24 (s, 2H) | 372 |
| 77 | 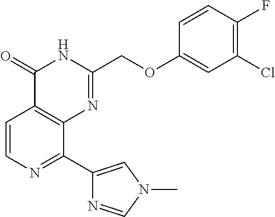 [SMILES: Cn1cnc(-c2nccc3c(=O)[nH]c(COc4ccc(F)c(Cl)c4)nc23)c1] | 2-((3-chloro-4- fluorophenoxy)methyl)-8- (1-methyl-1H-imidazol-4- yl)pyrido[3,4-d]pyrimidin- 4(3H)-one | Scheme C | ¹H NMR (400 MHz, DMSO- d₆) δ 12.78 (br s, 1H), 8.57 (d, J = 4.70 Hz, 1H), 8.03 (s, 1H), 7.70 (br d, J = 3.91 Hz, 1H), 7.31-7.47 (m, 2H), 7.14 (br s, 1H), 5.20 (s, 2H), 3.70 (s, 3H) | 386 |
| 78 | 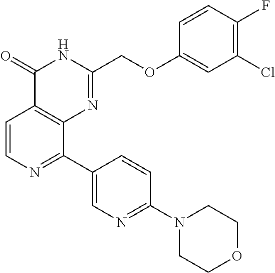 [SMILES: O=c1[nH]c(COc2ccc(F)c(Cl)c2)nc2c(-c3ccc(N4CCOCC4)nc3)nccc12] | 2-((3-chloro-4- fluorophenoxy)methyl)-8- (6-morpholinopyridin-3- yl)pyrido[3,4-d]pyrimidin- 4(3H)-one | Scheme C | ¹H NMR (400 MHz, DMSO- d₆) δ 12.80 (br s, 1H), 8.83 (d, J = 2.35 Hz, 1H), 8.64 (d, J = 5.09 Hz, 1H), 8.19 (br dd, J = 8.80, 2.15 Hz, 1H), 7.83 (d, J = 5.09 Hz, 1H), 7.25- 7.40 (m, 2H), 7.06 (dt, J = 9.29, 3.37 Hz, 1H), 6.75 (br d, J = 9.39 Hz, 1H), 5.09 (s, 2H), 3.60-3.81 (m, 2H), 3.49- 3.59 (m, 2H) | 468 |
| 79 | 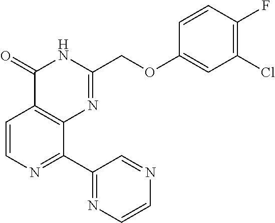 [SMILES: O=c1[nH]c(COc2ccc(F)c(Cl)c2)nc2c(-c3cnccn3)nccc12] | 2-((3-chloro-4- fluorophenoxy)methyl)-8- (pyrazin-2-yl)pyrido[3,4- d]pyrimidin-4(3H)-one | Scheme C | ¹H NMR (400 MHz, DMSO- d₆) δ 12.87 (br s, 1H), 8.90 (s, 1H), 8.69-8.81 (m, 2H), 8.65 (br s, 1H), 8.07 (d, J = 5.09 Hz, 1H), 7.20-7.35 (m, 2H), 6.99 (br d, J = 8.61 Hz, 1H), 5.01 (s, 2H) | 384 |
| 80 | 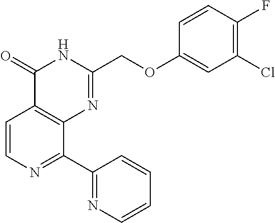 [SMILES: O=c1[nH]c(COc2ccc(F)c(Cl)c2)nc2c(-c3ccccn3)nccc12] | 2-((3-chloro-4- fluorophenoxy)methyl)-8- (pyridin-2-yl)pyrido[3,4- d]pyrimidin-4(3H)-one | Scheme C | ¹H NMR (400 MHz, CDCl₃) δ 8.73 (br d, J = 5.09 Hz, 1H), 8.58 (br s, 1H), 8.05- 8.14 (m, 1H), 7.94 (br t, J = 7.63 Hz, 1H), 7.39-7.46 (m, 1H), 7.01-7.18 (m, 2H), 6.88- 6.97 (m, 1H), 6.23 (q, J = 5.48 Hz, 2H), 5.09 (s, 2H) | 383 |
| 81 | 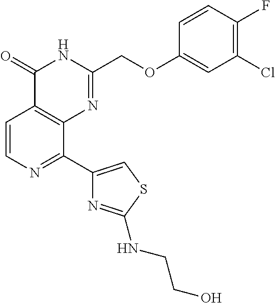 [SMILES: O=c1[nH]c(COc2ccc(F)c(Cl)c2)nc2c(-c3csc(NCCO)n3)nccc12] | 2-((3-chloro-4- fluorophenoxy)methyl)-8- (2-((2- hydroxyethyl)amino) thiazol-4-yl)pyrido[3,4- d]pyrimidin-4(3H)-one | Scheme C | ¹H NMR (400 MHz, CD₃OD) δ 8.67 (d, J = 5.09 Hz, 1H), 8.32 (s, 1H), 8.10 (d, J = 5.48 Hz, 1H), 7.99 (s, 1H), 7.57 (br t, J = 7.43 Hz, 1H), 7.44 (t, J = 7.83 Hz, 1H), 5.21 (s, 2H), 3.83 (t, J = 5.09 Hz, 2H), 3.62 (m, 2H) | 448 |
| 82 | 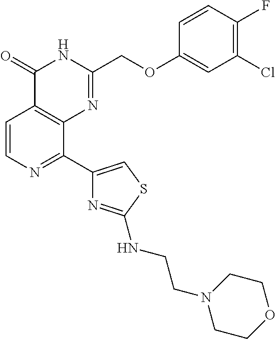 [SMILES: O=c1[nH]c(COc2ccc(F)c(Cl)c2)nc2c(-c3csc(NCCN4CCOCC4)n3)nccc12] | 2-((3-chloro-4- fluorophenoxy)methyl)-8- (2-((2- morpholinoethyl)amino) thiazol-4-yl)pyrido[3,4- d]pyrimidin-4(3H)-one | Scheme C | ¹H NMR (400 MHz, CD₃OD) δ 8.69 (d, J = 5.48 Hz, 1H), 8.31 (s, 1H), 8.07 (d, J = 5.09 Hz, 1H), 7.26 (dd, J = 5.87, 3.13 Hz, 1H), 7.17 (t, J = 9.00 Hz, 1H), 7.04 (dt, J = 9.39, 3.33 Hz, 1H), 5.21 (s, 2H), 4.01 (t, J = 4.89 Hz, 4H), 3.74-3.87 (m, 2H), 3.39- 3.55 (m, 6H) | 517 |
| 83 | 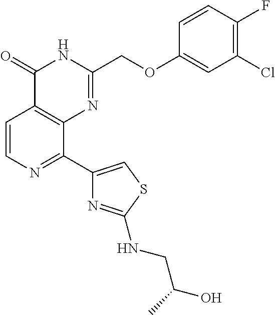 [SMILES: C[C@@H](O)CNc1nc(-c2nccc3c(=O)[nH]c(COc4ccc(F)c(Cl)c4)nc23)cs1] | (R)-2-((3-chloro-4- fluorophenoxy)methyl)-8- (2-((2- hydroxypropyl)amino) thiazol-4-yl)pyrido[3,4- d]pyrimidin-4(3H)-one | Scheme C | ¹H NMR (400 MHz, CD₃OD) δ 8.67 (d, J = 5.09 Hz, 1H), 8.27 (s, 1H), 8.09 (d, J = 5.09 Hz, 1H), 7.28 (dd, J = 5.87, 3.13 Hz, 1H), 7.19 (t, J = 8.80 Hz, 1H), 7.06 (dt, J = 9.10, 3.47 Hz, 1H), 5.22 (s, 2H), 3.51-3.67 (m, 2H), 3.38- 3.50 (m, 1H), 1.25 (d, J = 6.26 Hz, 3H) | 462 |
| 84 | 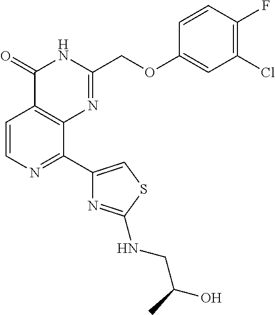 [SMILES: C[C@H](O)CNc1nc(-c2nccc3c(=O)[nH]c(COc4ccc(F)c(Cl)c4)nc23)cs1] | (S)-2-((3-chloro-4- fluorophenoxy)methyl)-8- (2-((2- hydroxypropyl)amino) thiazol-4-yl)pyrido[3,4- d]pyrimidin-4(3H)-one | Scheme C | ¹H NMR (400 MHz, CD₃OD) δ 8.67 (d, J = 5.09 Hz, 1H), 8.29 (s, 1H), 8.09 (d, J = 5.09 Hz, 1H), 7.28 (dd, J = 6.06, 2.93 Hz, 1H), 7.19 (t, J = 8.80 Hz, 1H), 7.06 (dt, J = 9.00, 3.33 Hz, 1H), 5.22 (s, 2H), 3.50-3.65 (m, 2H), 3.34- 3.49 (m, 1H), 1.25 (d, J = 6.65 Hz, 3H) | 462 |
| 85 | 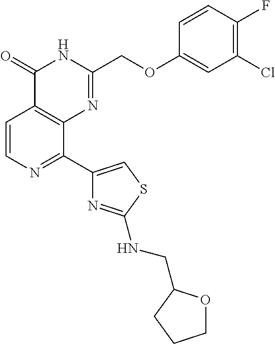 [SMILES: O=c1[nH]c(COc2ccc(F)c(Cl)c2)nc2c(-c3csc(NCC4CCCO4)n3)nccc12] | 2-((3-chloro-4- fluorophenoxy)methyl)-8- (2-(((tetrahydrofuran-2- yl)methyl)amino)thiazol- 4-yl)pyrido[3,4- d]pyrimidin-4(3H)-one | Scheme C | ¹H NMR (400 MHz, CD₃OD) δ 8.68 (d, J = 5.09 Hz, 1H), 8.31 (s, 1H), 8.10 (d, J = 5.09 Hz, 1H), 7.28 (dd, J = 5.87, 3.13 Hz, 1H), 7.19 (t, J = 9.00 Hz, 1H), 7.03-7.06 (m, 1H), 5.21 (s, 2H), 4.07-4.24 (m, 1H), 3.89-4.06 (m, 1H), 3.66-3.88 (m, 2H), 3.52 (dd, J = 14.28, 6.85 Hz, 1H), 2.10 (td, J = 12.62, 7.24 Hz, 1H), 1.90-2.03 (m, 2H), 1.63-1.76 (m, 1H) | 488 |
| 86 | 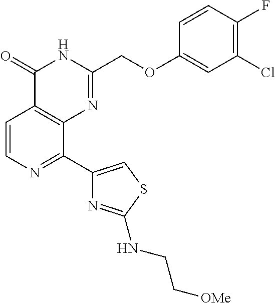 [SMILES: COCCNc1nc(-c2nccc3c(=O)[nH]c(COc4ccc(F)c(Cl)c4)nc23)cs1] | 2-((3-chloro-4- fluorophenoxy)methyl)-8- (2-((2- methoxyethyl)amino) thiazol-4-yl)pyrido[3,4- d]pyrimidin-4(3H)-one | Scheme C | ¹H NMR (400 MHz, CD₃OD) δ 8.68 (d, J = 5.48 Hz, 1H), 8.33 (s, 1H), 8.10 (d, J = 5.09 Hz, 1H), 7.29 (dd, J = 5.87, 3.13 Hz, 1H), 7.20 (t, J = 9.00 Hz, 1H), 7.06 (dt, J = 9.19, 3.23 Hz, 1H), 5.21 (s, 2H), 3.69 (s, 4H), 3.44 (s, 3H) | 462 |
| 87 | 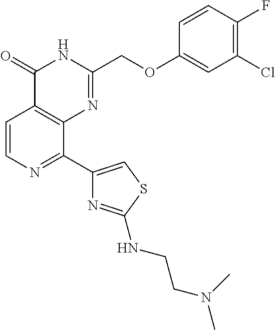 [SMILES: CN(C)CCNc1nc(-c2nccc3c(=O)[nH]c(COc4ccc(F)c(Cl)c4)nc23)cs1] | 2-((3-chloro-4- fluorophenoxy)methyl)-8- (2-((2- (dimethylamino)ethyl) amino)thiazol-4- yl)pyrido[3,4-d]pyrimidin- 4(3H)-one | Scheme C | ¹H NMR (400 MHz, CD₃OD) δ 8.69 (d, J = 5.48 Hz, 1H), 8.32 (s, 1H), 8.01 (d, J = 5.09 Hz, 1H), 7.27 (dd, J = 6.26, 3.13 Hz, 1H), 7.18 (t, J = 8.80 Hz, 1H), 7.05 (dt, J = 9.29, 3.37 Hz, 1H), 5.21 (s, 2H), 3.73-3.80 (m, 2H), 3.38- 3.54 (m, 2H). 3.00 (s, 6H) | 475 |
| 88 | 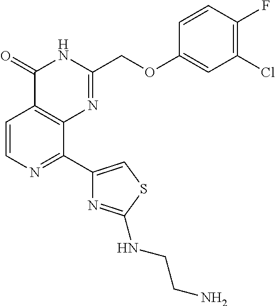 [SMILES: NCCNc1nc(-c2nccc3c(=O)[nH]c(COc4ccc(F)c(Cl)c4)nc23)cs1] | 8-(2-((2- aminoethyl)amino)thiazol- 4-yl)-2-((3-chloro-4- fluorophenoxy)methyl) pyrido[3,4-d]pyrimidin- 4(3H)-one | Scheme C | ¹H NMR (400 MHz, CD₃OD) δ 8.67 (d, J = 5.48 Hz, 1H), 8.50 (s, 1H), 8.18 (d, J = 5.87 Hz, 1H), 7.28 (dd, J = 6.06, 2.93 Hz, 1H), 7.19 (t, J = 9.00 Hz, 1H), 7.06 (dt, J = 9.19, 3.23 Hz, 1H), 5.24 (s, 2H), 3.78 (t, J = 5.48 Hz, 2H), 3.12-3.28 (m, 2H) | 447 |
| 89 | 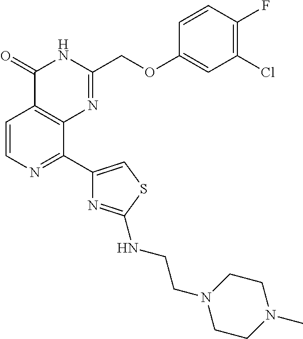 [SMILES: CN1CCN(CCNc2nc(-c3nccc4c(=O)[nH]c(COc5ccc(F)c(Cl)c5)nc34)cs2)CC1] | 2-((3-chloro-4- fluorophenoxy)methyl)-8- (2-((2-(4-methylpiperazin- 1-yl)ethyl)amino)thiazol- 4-yl)pyrido[3,4- d]pyrimidin-4(3H)-one | Scheme C | ¹H NMR (400 MHz, CD₃OD) δ 8.71 (d, J = 5.48 Hz, 1H), 8.41 (s, 1H), 8.13 (d, J = 5.48 Hz, 1H), 7.29 (dd, J = 6.06, 2.93 Hz, 1H), 7.20 (t, J = 8.80 Hz, 1H), 7.07 (dt, J = 9.10, 3.47 Hz, 1H), 5.22 (s, 2H), 3.67 (br t, J = 5.48 Hz, 2H), 3.32-3.45 (m, 4H), 2.95- 3.01 (m, 2H), 2.92 (s, 3H) | 530 |
| 90 | 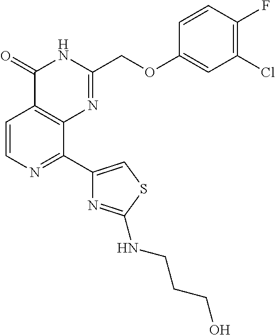 [SMILES: O=c1[nH]c(COc2ccc(F)c(Cl)c2)nc2c(-c3csc(NCCCO)n3)nccc12] | 2-((3-chloro-4- fluorophenoxy)methyl)-8- (2-((3- hydroxypropyl)amino) thiazol-4-yl)pyrido[3,4- d]pyrimidin-4(3H)-one | Scheme C | ¹H NMR (400 MHz, CD₃OD) δ 8.69 (d, J = 8.00 Hz, 1H), 8.35 (s, 1H), 8.10 (d, J = 5.09 Hz, 1H), 7.29 (dd, J = 5.87, 3.13 Hz, 1H), 7.20 (t, J = 9.00 Hz, 1H), 7.07 (dt, J = 9.10, 3.47 Hz, 1H), 5.21 (s, 2H), 4.52 (br t, J = 6.26 Hz, 1H), 3.71 (t, 2H), 3.63 (t, 2H), 1.94 (m, 2H) | 462 |
| 91 | 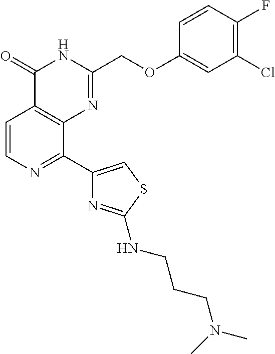 [SMILES: CN(C)CCCNc1nc(-c2nccc3c(=O)[nH]c(COc4ccc(F)c(Cl)c4)nc23)cs1] | 2-((3-chloro-4- fluorophenoxy)methyl)-8- (2-((3- (dimethylamino)propyl) amino)thiazol-4- yl)pyrido[3,4-d]pyrimidin- 4(3H)-one | Scheme C | ¹H NMR (400 MHz, CD₃OD) δ 8.67 (d, J = 5.09 Hz, 1H), 8.40 (s, 1H), 8.10 (d, J = 5.09 Hz, 1H), 7.28 (dd, J = 5.87, 3.13 Hz, 1H), 7.19 (t, J = 8.80 Hz, 1H), 7.06 (dt, J = 9.19, 3.23 Hz, 1H), 5.22 (s, 2H), 3.61 (t, J = 6.65 Hz, 2H), 3.21-3.28 (m, 2H), 2.83- 2.95 (m, 6H), 2.14 (quin, J = 7.04 Hz, 2H) | 489 |
| 92 | 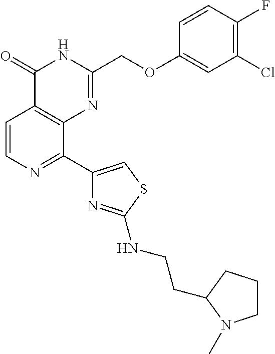 [SMILES: CN1CCCC1CCNc1nc(-c2nccc3c(=O)[nH]c(COc4ccc(F)c(Cl)c4)nc23)cs1] | 2-((3-chloro-4- fluorophenoxy)methyl)-8- (2-((2-(1- methylpyrrolidin-2- yl)ethyl)amino)thiazol-4- yl)pyrido[3,4-d]pyrimidin- 4(3H)-one | Scheme C | ¹H NMR (400 MHz, CD₃OD) δ 8.66 (d, J = 5.48 Hz, 1H), 8.40 (s, 1H), 8.12 (d, J = 5.09 Hz, 1H), 7.28 (dd, J = 5.87, 3.13 Hz, 1H), 7.18 (t, J = 9.00 Hz, 1H), 7.06 (dt, J = 9.10, 3.47 Hz, 1H), 5.23 (s, 2H), 3.84 (t, J = 5.28 Hz, 2H), 3.62 (br s, 2H), 3.40- 3.47 (m, 2H), 3.05 (br s, 2H), 1.81-2.01 (m, 6H) | 515 |
| 93 | 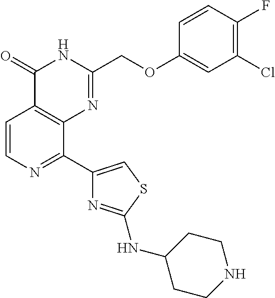 [SMILES: O=c1[nH]c(COc2ccc(F)c(Cl)c2)nc2c(-c3csc(NC4CCNCC4)n3)nccc12] | 2-((3-chloro-4- fluorophenoxy)methyl)-8- (2-(piperidin-4- ylamino)thiazol-4- yl)pyrido[3,4-d]pyrimidin- 4(3H)-one | Scheme C | ¹H NMR (400 MHz, CD₃OD) δ 8.63 (d, J = 5.48 Hz, 1H), 8.51 (s, 1H), 8.21 (d, J = 5.48 Hz, 1H), 7.29 (dd, J = 5.87, 3.13 Hz, 1H), 7.19 (t, J = 9.00 Hz, 1H), 7.07 (dt, J = 9.19, 3.23 Hz, 1H), 5.25 (s, 2H), 4.05-4.19 (m, 1H), 3.42- 3.55 (m, 2H), 3.09-3.24 (m, 2H), 2.28-2.46 (m, 2H), 1.75- 1.94 Scheme C (m, 2H) | 487 |
| 94 | 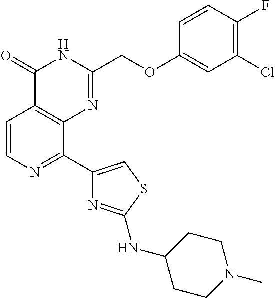 [SMILES: CN1CCC(Nc2nc(-c3nccc4c(=O)[nH]c(COc5ccc(F)c(Cl)c5)nc34)cs2)CC1] | 2-((3-chloro-4- fluorophenoxy)methyl)-8- (2-((1-methylpiperidin-4- yl)amino)thiazol-4- yl)pyrido[3,4-d]pyrimidin- 4(3H)-one | Scheme C | ¹H NMR (400 MHz, CD₃OD) δ 8.63 (d, J = 5.48 Hz, 1H), 8.51 (s, 1H), 8.20 (d, J = 5.48 Hz, 1H), 7.28 (dd, J = 6.06, 2.93 Hz, 1H), 7.19 (t, J = 8.80 Hz, 1H), 7.06 (dt, J = 9.10, 3.47 Hz, 1H), 5.24 (s, 2H), 3.93-4.17 (m, 1H), 3.64 (br d, J = 12.62 Hz, 2H), 3.12-3.22 (m, 2H), 2.91 (s, 3H), 2.44 (br d, J = 13.30 Hz, 2H), 1.77-1.94 (m, 2H) | 501 |
| 95 | 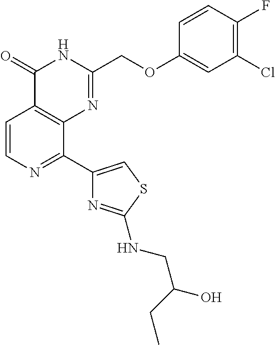 [SMILES: CCC(O)CNc1nc(-c2nccc3c(=O)[nH]c(COc4ccc(F)c(Cl)c4)nc23)cs1] | 2-((3-chloro-4- fluorophenoxy)methyl)-8- (2-((2- hydroxybutyl)amino) thiazol-4-yl)pyrido[3,4- d]pyrimidin-4(3H)-one | Scheme C | none | 476 |
| 96 | 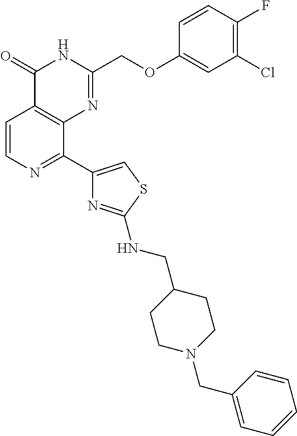 [SMILES: O=c1[nH]c(COc2ccc(F)c(Cl)c2)nc2c(-c3csc(NCC4CCN(Cc5ccccc5)CC4)n3)nccc12] | 8-(2-(((1-benzylpiperidin- 4- yl)methyl)amino)thiazol- 4-yl)-2-((3-chloro-4- fluorophenoxy)methyl) pyrido[3,4-d]pyrimidin- 4(3H)-one | Scheme C | none | 591 |
| 97 | 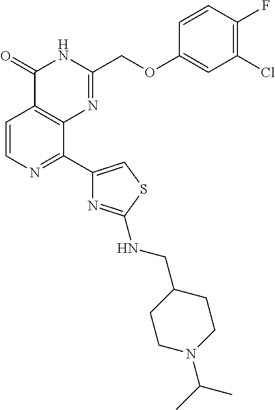 [SMILES: CC(C)N1CCC(CNc2nc(-c3nccc4c(=O)[nH]c(COc5ccc(F)c(Cl)c5)nc34)cs2)CC1] | 2-((3-chloro-4- fluorophenoxy)methyl)-8- (2-(((1-isopropylpiperidin- 4- yl)methyl)amino)thiazol- 4-yl)pyrido[3,4- d]pyrimidin-4(3H)-one | Scheme C | none | 543 |
| 98 | 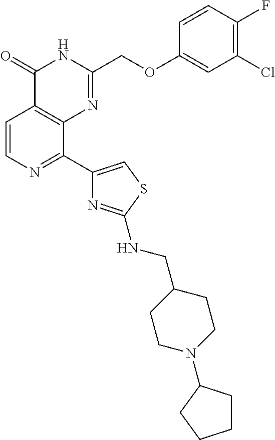 [SMILES: O=c1[nH]c(COc2ccc(F)c(Cl)c2)nc2c(-c3csc(NCC4CCN(C5CCCC5)CC4)n3)nccc12] | 2-((3-chloro-4- fluorophenoxy)methyl)-8- (2-(((1- cyclopentylpiperidin-4- yl)methyl)amino)thiazol- 4-yl)pyrido[3,4- d]pyrimidin-4(3H)-one | Scheme C | none | 569 |
| 99 | 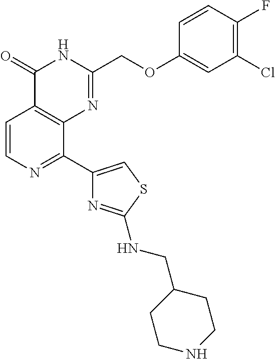 [SMILES: O=c1[nH]c(COc2ccc(F)c(Cl)c2)nc2c(-c3csc(NCC4CCNCC4)n3)nccc12] | 2-((3-chloro-4- fluorophenoxy)methyl)-8- (2-((piperidin-4- ylmethyl)amino)thiazol-4- yl)pyrido[3,4-d]pyrimidin- 4(3H)-one | Scheme C | none | 501 |
| 100 | 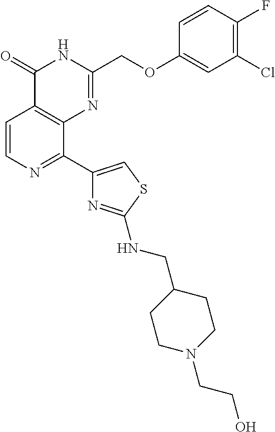 [SMILES: O=c1[nH]c(COc2ccc(F)c(Cl)c2)nc2c(-c3csc(NCC4CCN(CCO)CC4)n3)nccc12] | 2-((3-chloro-4- fluorophenoxy)methyl)-8- (2-(((1-(2- hydroxyethyl)piperidin-4- yl)methyl)amino)thiazol- 4-yl)pyrido[3,4- d]pyrimidin-4(3H)-one | Scheme C | none | 545 |
| 101 | 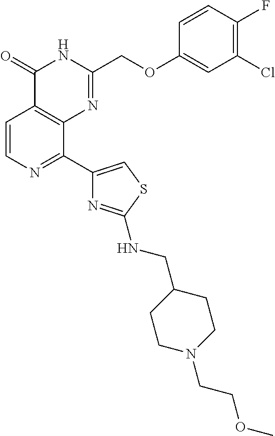 [SMILES: COCCN1CCC(CNc2nc(-c3nccc4c(=O)[nH]c(COc5ccc(F)c(Cl)c5)nc34)cs2)CC1] | 2-((3-chloro-4- fluorophenoxy)methyl)-8- (2-(((1-(2- methoxyethyl)piperidin-4- yl)methyl)amino)thiazol- 4-yl)pyrido[3,4- d]pyrimidin-4(3H)-one | Scheme C | none | 559 |
| 102 | 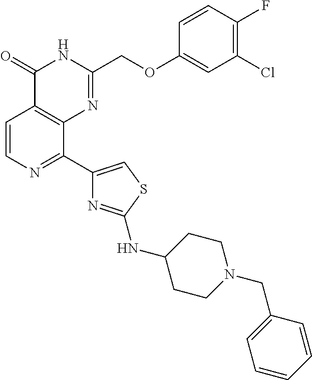 [SMILES: O=c1[nH]c(COc2ccc(F)c(Cl)c2)nc2c(-c3csc(NC4CCN(Cc5ccccc5)CC4)n3)nccc12] | 8-(2-((1-benzylpiperidin- 4-yl)amino)thiazol-4-yl)- 2-((3-chloro-4- fluorophenoxy)methyl) pyrido[3,4-d]pyrimidin- 4(3H)-one | Scheme C | ¹H NMR (400 MHz, DMSO- d₆) δ 9.55 (s, 1H), 8.65 (d, 1H), 7.90 (d, 1H), 7.71 (s, 1H), 7.48 (s, 4H), 7.37 (m, 2H), 7.11 (m, 1H), 5.19 (s, 2H), 4.29 (m, 2H), 3.45 (d, 2H), 3.06 (m, 1H), 2.22 (d, 2H), 1.66 (m, 2H) | 577 |
| 103 | 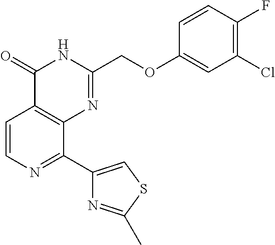 [SMILES: Cc1nc(-c2nccc3c(=O)[nH]c(COc4ccc(F)c(Cl)c4)nc23)cs1] | 2-((3-chloro-4- fluorophenoxy)methyl)-8- (2-methylthiazol-4- yl)pyrido[3,4-d]pyrimidin- 4(3H)-one | Scheme C | ¹H NMR (400 MHz, CD₃OD) δ 8.99 (s, 1H), 8.67 (d, 1H), 8.26 (d, J = 5.09 Hz, 1H), 7.32 (m, 1H), 7.21 (m, 1H), 7.10 (m, 1H), 5.27 (s, 2H), 2.86 (s, 3H) | 403 |
| 104 | 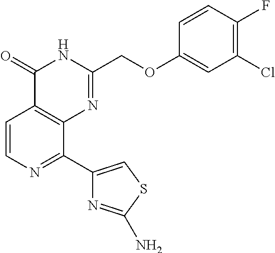 [SMILES: Nc1nc(-c2nccc3c(=O)[nH]c(COc4ccc(F)c(Cl)c4)nc23)cs1] | 8-(2-aminothiazol-4-yl)-2- ((3-chloro-4- fluorophenoxy)methyl) pyrido[3,4-d]pyrimidin- 4(3H)-one | Scheme C | ¹H NMR (400 MHz, CD₃OD) δ 8.70 (d, J = 5.09 Hz, 1H), 8.31 (s, 1H), 8.09 (d, J = 5.09 Hz, 1H), 7.29 (dd, J = 6.26, 3.13 Hz, 1H), 7.20 (t, J = 9.00, 9.00 Hz, 1H), 7.07 (m, 1H), 5.22 (s, 2H), | 404 |
| 105 | 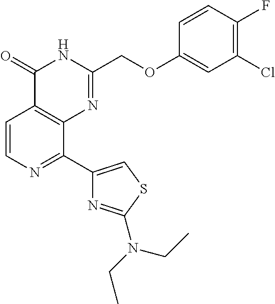 [SMILES: CCN(CC)c1nc(-c2nccc3c(=O)[nH]c(COc4ccc(F)c(Cl)c4)nc23)cs1] | 2-((3-chloro-4- fluorophenoxy)methyl)-8- (2-(diethylamino)thiazol- 4-yl)pyrido[3,4- d]pyrimidin-4(3H)-one | Scheme C | ¹H NMR (400 MHz, CD₃OD) δ 8.79 (d, J = 5.09 Hz, 1H), 8.23 (d, J = 5.09 Hz, 1H), 7.24 (s, 1H), 7.20-7.14 (m, 2H), 6.99 (m, 1H), 5.09 (s, 2H), 4.02 (q, J = 7.30, 7.30, 7.30 Hz, 2H), 3.54 (q, J = 7.30, 7.30, 7.30, 7.30 Hz, 2H), 1.45 (t, J = 7.24, 7.24 Hz, 3H), 1.17 (t, J = 7.24, 7.24 Hz, 3H) | 460 |
| 106 | 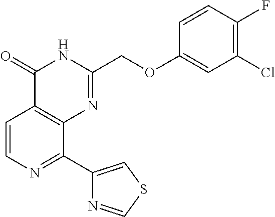 [SMILES: O=c1[nH]c(COc2ccc(F)c(Cl)c2)nc2c(-c3cscn3)nccc12] | 2-((3-chloro-4- fluorophenoxy)methyl)-8- (thiazol-4-yl)pyrido[3,4- d]pyrimidin-4(3H)-one | Scheme C | ¹H NMR (400 MHz, CD₃OD) δ 8.69 (d, J = 5.09 Hz, 1H), 8.34 (s, 1H), 8.11 (d, J = 5.09 Hz, 1H), 6.80 (dd, J = 8.80, 2.15 Hz, 1H) 6.63 (br t, J = 9.19, 9.19 Hz, 1H), 5.25 (s, 2H), 3.84 (t, 1H), 3.64 (t, 1H) | 389 |
| 107 | 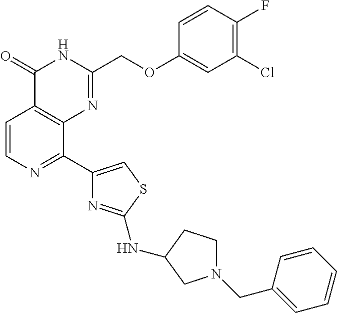 [SMILES: O=c1[nH]c(COc2ccc(F)c(Cl)c2)nc2c(-c3csc(NC4CCN(Cc5ccccc5)C4)n3)nccc12] | 8-(2-((1-benzylpyrrolidin- 3-yl)amino)thiazol-4-yl)- 2-((3-chloro-4- fluorophenoxy)methyl) pyrido[3,4-d]pyrimidin- 4(3H)-one | Scheme C | ¹H NMR (400 MHz, CD₃OD) δ 8.76 (s, 1H), 8.35 (s, 1H), 8.07 (s, 1H), 7.44 (m, 2H), 7.37 (m, 3H), 7.29 (dd, J = 6.06, 2.93 Hz, 1H), 7.19 (t, 1H), 7.07 (m, 1H), 5.24 (s, 2H), 4.44 (m, 1H), 4.35 (m, 1H), 4.03 (m, 1H), 3.43 (m, 2H), 2.63 (m, 1H), 2.29 (m, 1H) | 563 |
| 108 | 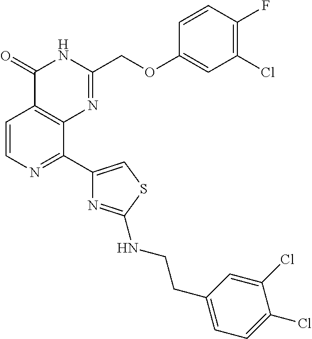 [SMILES: O=c1[nH]c(COc2ccc(F)c(Cl)c2)nc2c(-c3csc(NCCc4ccc(Cl)c(Cl)c4)n3)nccc12] | 2-((3-chloro-4- fluorophenoxy)methyl)-8- (2-((3,4- dichlorophenethyl)amino) thiazol-4-yl)pyrido[3,4- d]pyrimidin-4(3H)-one | Scheme C | ¹H NMR (400 MHz, CD₃OD) δ 8.66 (d, J = 5.09 Hz, 1H), 8.32 (s, 1H), 8.12 (d, J = 5.09 Hz, 1H), 7.50 (d, 1H), 7.43 (d, J = 8.61 Hz, 1H), 7.28 (m, 1H), 7.24-7.28 (m, 2H), 7.08- 7.04 (m, 1H), 5.21 (s, 2H), 3.77 (t, J = 6.85, 6.85 Hz, 2H), 3.01 (t, 2H) | 577 |
| 109 | 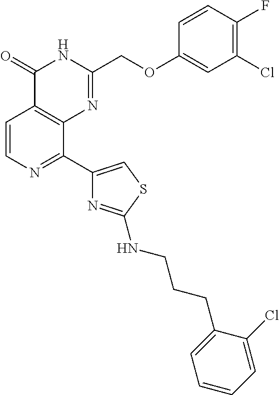 [SMILES: O=c1[nH]c(COc2ccc(F)c(Cl)c2)nc2c(-c3csc(NCCCc4ccccc4Cl)n3)nccc12] | 2-((3-chloro-4- fluorophenoxy)methyl)-8- (2-((3-(2- chlorophenyl)propyl) amino)thiazol-4-yl)pyrido[3,4- d]pyrimidin-4(3H)-one | Scheme C | ¹H NMR (400 MHz, CD₃OD) δ 8.68 (d, J = 5.09 Hz, 1H), 8.37 (s, 1H), 8.13 (d, J = 5.09 Hz, 1H), 7.27 (m, 2H), 7.18 (t, 1H), 7.09-7.03 (m, 3H), 6.94 (m, 1H), 5.21 (s, 2H), 4.32 (t, 2H), 4.01 (t, 2H), 3.24 (m, 2H) | 556 |
| 110 | 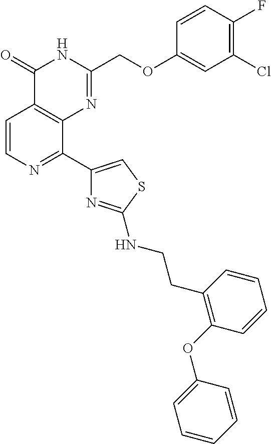 [SMILES: O=c1[nH]c(COc2ccc(F)c(Cl)c2)nc2c(-c3csc(NCCc4ccccc4Oc4ccccc4)n3)nccc12] | 2-((3-chloro-4- fluorophenoxy)methyl)-8- (2-((2- phenoxyphenethyl)amino) thiazol-4-yl)pyrido[3,4- d]pyrimidin-4(3H)-one | Scheme C | ¹H NMR (400 MHz, CD₃OD) δ 8.67 (d, J = 5.09 Hz, 1H), 8.23 (s, 1H), 8.09 (d, J = 5.48 Hz, 1H), 7.40 (d, J = 5.87 Hz, 1H), 7.28-7.23 (m, 4H), 7.18 (t, 1H), 7.12 (t, 1H), 7.08- 6.09 (m, 2H), 6.91 (d, J = 7.83 Hz, 2H), 6.85 (d, 1H), 5.19 (s, 2H), 3.77 (t, J = 6.85, 6.85 Hz, 2H), 3.08 (t, J = 6.85, 6.85 Hz, 2H) | 600 |
| 111 | 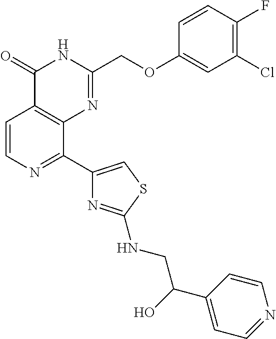 [SMILES: O=c1[nH]c(COc2ccc(F)c(Cl)c2)nc2c(-c3csc(NCC(O)c4ccncc4)n3)nccc12] | 2-((3-chloro-4- fluorophenoxy)methyl)-8- (2-((2-hydroxy-2-(pyridin- 4-yl)ethyl)amino)thiazol- 4-yl)pyrido[3,4- d]pyrimidin-4(3H)-one | Scheme C | ¹H NMR (400 MHz, CD₃OD) δ 8.77 (d, J = 6.65 Hz, 2H), 8.67 (d, J = 5.48 Hz, 1H), 8.36 (s, 1H), 8.14 (d, J = 5.48 Hz, 1H), 8.08 (d, 2H), 7.28 (m, 1H), 7.19 (t, 1H), 7.06 (m, 1H), 5.28 (m, 1H), 5.23 (s, 2H), 3.97 (m, 1H), 3.81 (m, 1H) | 525 |
| 112 | 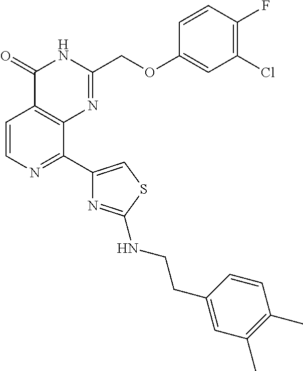 [SMILES: Cc1ccc(CCNc2nc(-c3nccc4c(=O)[nH]c(COc5ccc(F)c(Cl)c5)nc34)cs2)cc1C] | 2-((3-chloro-4- fluorophenoxy)methyl)-8- (2-((3,4- dimethylphenethyl)amino) thiazol-4-yl)pyrido[3,4- d]pyrimidin-4(3H)-one | Scheme C | ¹H NMR (400 MHz, CD₃OD) δ 8.68 (d, J = 5.09 Hz, 1H), 8.26 (s, 1H), 8.10 (d, J = 5.09 Hz, 1H), 7.27 (m, 1H), 7.19 (t, 1H), 7.09-6.97 (m, 4H), 5.20 (s, 2H), 3.73 (t, 2H), 2.95 (t, 2H), 2.20 (s, 3H), 2.16 (s, 3H) | 536 |
| 113 | 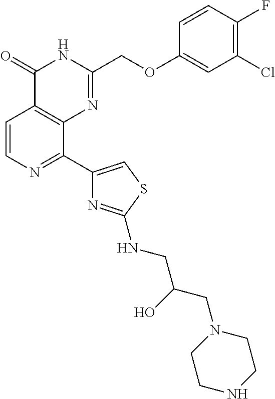 [SMILES: O=c1[nH]c(COc2ccc(F)c(Cl)c2)nc2c(-c3csc(NCC(O)CN4CCNCC4)n3)nccc12] | 2-((3-chloro-4- fluorophenoxy)methyl)-8- (2-((2-hydroxy-3- (piperazin-1- yl)propyl)amino)thiazol-4- yl)pyrido[3,4-d]pyrimidin- 4(3H)-one | Scheme C | ¹H NMR (400 MHz, CD₃OD) δ 8.67 (d, J = 5.48 Hz, 1H), 8.41 (s, 1H), 8.14 (d, J = 5.09 Hz, 1H), 7.29 (m, 1H), 7.19 (t, 1H), 7.08 (m, 1H), 5.24 (s, 2H), 4.29 (m, 1H), 3.75-3.55 (m, 3H), 3.46 (m, 3H), 3.33 (m, 3H), 3.08 (m, 2H), 1.30 (t, 1H) | 546 |
| 114 | 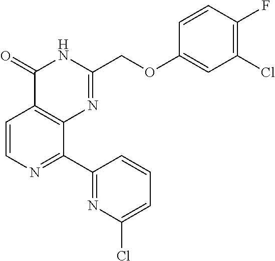 [SMILES: O=c1[nH]c(COc2ccc(F)c(Cl)c2)nc2c(-c3cccc(Cl)n3)nccc12] | 2-((3-chloro-4- fluorophenoxy)methyl)-8- (6-chloropyridin-2- yl)pyrido[3,4-d]pyrimidin- 4(3H)-one | Scheme C | ¹H NMR (400 MHz, CD₃OD) δ 7.70 (m, 2H), 7.45 (m, 1H), 7.30 (m, 2H), 7.22 (m, 1H), 7.11 (m, 2H) | 417 |
| 115 | 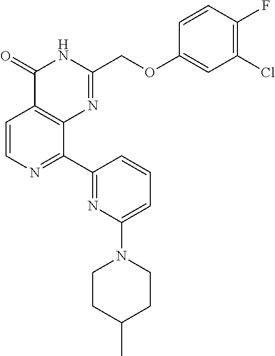 [SMILES: CC1CCN(c2cccc(-c3nccc4c(=O)[nH]c(COc5ccc(F)c(Cl)c5)nc34)n2)CC1] | 2-((3-chloro-4- fluorophenoxy)methyl)-8- (6-(4-methylpiperidin-1- yl)pyridin-2-yl)pyrido[3,4- d]pyrimidin-4(3H)-one | Scheme C | ¹H NMR (400 MHz, CD₃OD) δ 8.88 (d, 1H), 8.29 (d, 1H), 8.27 (d, 1H), 8.11 (m, 1H), 7.49 (d, 1H), 7.27 (m, 2H), 7.08 (m, 1H), 5.18 (s, 2H), 4.25 (d, 2H), 3.38 (m, 2H), 1.85 (d, 2H), 1.38 (m, 3H), 1.00 (d, 3H) | 480 |
| 116 | 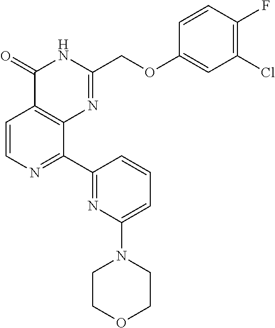 [SMILES: O=c1[nH]c(COc2ccc(F)c(Cl)c2)nc2c(-c3cccc(N4CCOCC4)n3)nccc12] | 2-((3-chloro-4- fluorophenoxy)methyl)-8- (6-morpholinopyridin-2- yl)pyrido[3,4-d]pyrimidin- 4(3H)-one | Scheme C | ¹H NMR (400 MHz, CD₃OD) δ 8.87 (d, J = 5.09 Hz, 1H), 8.39 (d, J = 7.43 Hz, 1H), 8.28 (d, J = 5.09 Hz, 1H), 8.16 (m, 1H), 7.50 (d, J = 9.39 Hz, 1H), 7.28 (m, 1H), 7.23 (d, 1H), 7.07 (m, 1H), 5.19 (s, 2H), 3.85 (m, 4H), 3.79 (m, 4H) | 468 |
| 117 | 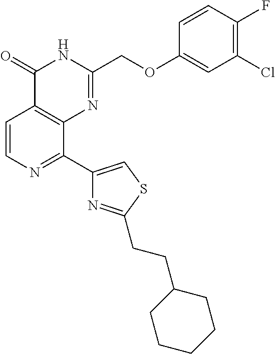 [SMILES: O=c1[nH]c(COc2ccc(F)c(Cl)c2)nc2c(-c3csc(CCC4CCCCC4)n3)nccc12] | 2-((3-chloro-4- fluorophenoxy)methyl)-8- (2- ((cyclohexylmethyl)amino) thiazol-4-yl)pyrido[3,4- d]pyrimidin-4(3H)-one | Scheme C | ¹H NMR (400 MHz, CD₃OD) δ 8.69 (d, J = 5.48 Hz, 1H), 8.30 (s, 1H), 8.09 (d, J = 5.09 Hz, 1H), 7.28 (dd, 1H), 7.20 (t, 1H), 7.09-7.05 (m, 1H), 5.21 (s, 2H), 3.33 (d, 2H), 1.88-165 (m, 5H), 1.31 (m, 3H), 1.09 (m, 3H) | 500 |
| 118 | 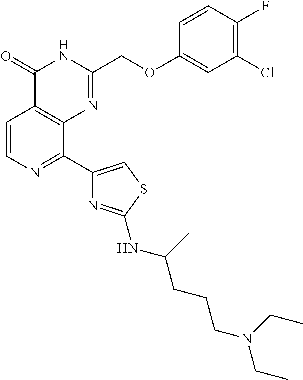 [SMILES: CCN(CC)CCCC(C)Nc1nc(-c2nccc3c(=O)[nH]c(COc4ccc(F)c(Cl)c4)nc23)cs1] | 2-((3-chloro-4- fluorophenoxy)methyl)-8- (2-((5- (diethylamino)pentan-2- yl)amino)thiazol-4- yl)pyrido[3,4-d]pyrimidin- 4(3H)-one | Scheme C | ¹H NMR (400 MHz, CD₃OD) δ 8.67 (d, J = 5.09 Hz, 1H), 8.33 (s, 1H), 8.12 (d, J = 5.09 Hz, 1H), 7.28-7.17 (m, 2H), 7.05 (m, 1H), 6.93 (m, 1H), 5.20 (s, 2H), 4.78 (m, 1H), | 545 |
| 119 | 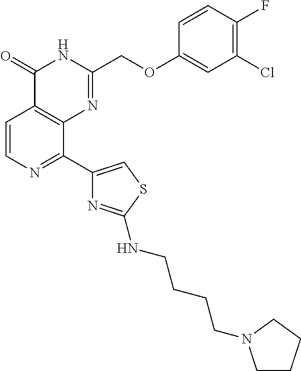 [SMILES: O=c1[nH]c(COc2ccc(F)c(Cl)c2)nc2c(-c3csc(NCCCCN4CCCC4)n3)nccc12] | 2-((3-chloro-4- fluorophenoxy)methyl)-8- (2-((4-(pyrrolidin-1- yl)butyl)amino)thiazol-4- yl)pyrido[3,4-d]pyrimidin- 4(3H)-one | Scheme C | ¹H NMR (400 MHz, CD₃OD) δ 8.68 (d, J = 5.09 Hz, 1H), 8.33 (s, 1H), 8.12 (d, J = 5.48 Hz, 1H), 7.29 (m, 1H), 7.21 (t, 1H), 7.08 (m, 1H), 5.23 (s, 2H), 3.91 (m, 1H), 3.25-3.16 (m, 5H), 1.91-1.72 (m, 4H), 1.38 (d, J = 6.65 Hz, 2H), 1.29 (t, J = 7.43, 7.43 Hz, 4H) | 529 |
| 120 | 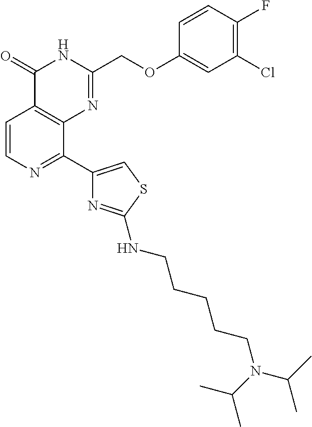 [SMILES: CC(C)N(CCCCCNc1nc(-c2nccc3c(=O)[nH]c(COc4ccc(F)c(Cl)c4)nc23)cs1)C(C)C] | 2-((3-chloro-4- fluorophenoxy)methyl)-8- (2-((5- (diisopropylamino)pentyl) amino)thiazol-4- yl)pyrido[3,4-d]pyrimidin- 4(3H)-one | Scheme C | ¹H NMR (400 MHz, CD₃OD) δ 8.68 (d, J = 5.48 Hz, 1H), 8.26 (s, 1H), 8.10 (d, J = 5.09 Hz, 1H), 7.30 (m, 1H), 7.21 (t, 1H), 7.09 (m, 1H), 5.22 (s, 2H), 3.52 (m, 2H), 2.15 (m, 2H), 2.03 (m, 2H), 1.82 (m, 4H) | 573 |
| 121 | 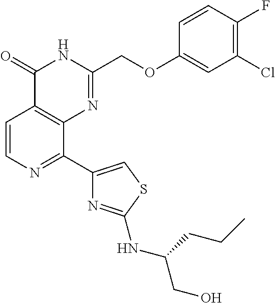 [SMILES: CCC[C@H](CO)Nc1nc(-c2nccc3c(=O)[nH]c(COc4ccc(F)c(Cl)c4)nc23)cs1] | (R)-2-((3-chloro-4- fluorophenoxy)methyl)-8- (2-((1-hydroxypentan-2- yl)amino)thiazol-4- yl)pyrido[3,4-d]pyrimidin- 4(3H)-one | Scheme C | ¹H NMR (400 MHz, CD₃OD) δ 8.68 (d, J = 5.48 Hz, 1H), 8.32 (s, 1H), 8.10 (d, J = 5.48 Hz, 1H), 7.28 (m, 1H), 7.20 (t, 1H), 7.08 (m, 1H), 5.22 (s, 2H), 3.73 (m, 2H), 3.51 (m, 1H), 3.13 (m, 2H), 1.80 (m, 2H), 1.56 (m, 1H), 1.37 (m, 5H) | 490 |
| 122 | 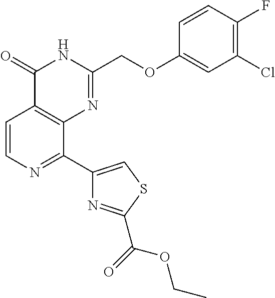 [SMILES: CCOC(=O)c1nc(-c2nccc3c(=O)[nH]c(COc4ccc(F)c(Cl)c4)nc23)cs1] | ethyl 4-(2-((3-chloro-4- fluorophenoxy)methyl)-4- oxo-3,4- dihydropyrido[3,4- d]pyrimidin-8-yl)thiazole- 2-carboxylate | Scheme C | ¹H NMR (400 MHz, CD₃OD) δ 8.69 (d, J = 5.09 Hz, 1H), 8.29 (s, 1H), 8.11 (d, J = 5.09 Hz, 1H), 7.29 (m, 1H), 7.21 (t, 1H), 7.09 (m, 1H), 5.23 (s, 2H), 3.67 (m, 2H), 1.02 (t, 3H) | 461 |
| 123 | 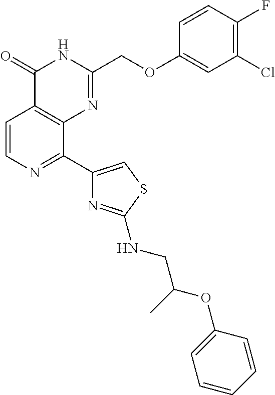 [SMILES: CC(CNc1nc(-c2nccc3c(=O)[nH]c(COc4ccc(F)c(Cl)c4)nc23)cs1)Oc1ccccc1] | 2-((3-chloro-4- fluorophenoxy)methyl)-8- (2-((2- phenoxypropyl)amino) thiazol-4-yl)pyrido[3,4- d]pyrimidin-4(3H)-one | Scheme C | ¹H NMR (400 MHz, CD₃OD) δ 8.97 (s, 1H), 8.72 (d, 1H), 8.10 (d, 1H), 7.32 (m, 1H), 7.19 (m, 2H), 7.10 (m, 2H), 5.20 (s, 2H), 4.50 (m, 2H), 3.24 (m, 1H), 1.44 (m, 2H), 0.94 (t, 3H) | 538 |
| 124 | 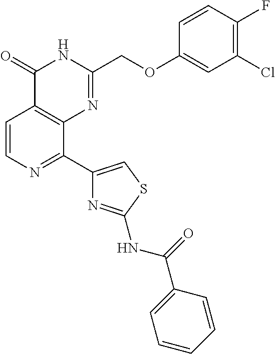 [SMILES: O=C(Nc1nc(-c2nccc3c(=O)[nH]c(COc4ccc(F)c(Cl)c4)nc23)cs1)c1ccccc1] | N-(4-(2-((3-chloro-4- fluorophenoxy)methyl)-4- oxo-3,4- dihydropyrido[3,4- d]pyrimidin-8-yl)thiazol- 2-yl)benzamide | Scheme C | ¹H NMR (400 MHz, CD₃OD) δ 8.77 (s, 1H), 8.67 (d, 1H), 8.18 (d, 1H), 8.06 (d, 2H), 7.67 (d, 1H), 7.58 (t, 2H), 7.32 (m, 1H), 7.20 (t, 1H), 7.10 (m, 1H), 5.26 (s, 2H) | 508 |
| 125 | 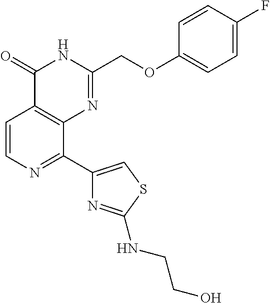 [SMILES: O=c1[nH]c(COc2ccc(F)cc2)nc2c(-c3csc(NCCO)n3)nccc12] | 2-((4- fluorophenoxy)methyl)-8- (2-((2- hydroxyethyl)amino) thiazol-4-yl)pyrido[3,4- d]pyrimidin-4(3H)-one | Scheme C | ¹H NMR (400 MHz, CD₃OD) δ 8.96 (s, 1H), 8.77 (d, 1H), 8.65 (s, 1H), 8.57 (d, 1H), 8.39 (d, 1H), 8.25 (s, 1H), 8.10 (s, 1H), 6.23 (m, 2H), 3.86 (m, 2H), 3.48 (t, 2H), | 413 |
| 126 | 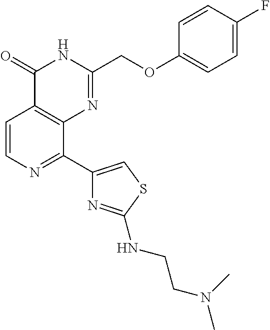 [SMILES: CN(C)CCNc1nc(-c2nccc3c(=O)[nH]c(COc4ccc(F)cc4)nc23)cs1] | 8-(2-((2- (dimethylamino)ethyl) amino)thiazol-4-yl)-2-((4- fluorophenoxy)methyl) pyrido[3,4-d]pyrimidin- 4(3H)-one | Scheme C | none | 440 |
| 127 | 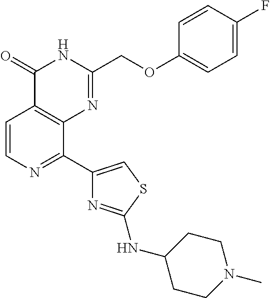 [SMILES: CN1CCC(Nc2nc(-c3nccc4c(=O)[nH]c(COc5ccc(F)cc5)nc34)cs2)CC1] | 2-((4- fluorophenoxy)methyl)-8- (2-((1-methylpiperidin-4- yl)amino)thiazol-4- yl)pyrido[3,4-d]pyrimidin- 4(3H)-one | Scheme C | none | 467 |
| 128 | 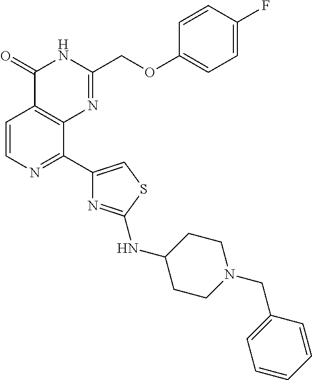 [SMILES: O=c1[nH]c(COc2ccc(F)cc2)nc2c(-c3csc(NC4CCN(Cc5ccccc5)CC4)n3)nccc12] | 8-(2-((1-benzylpiperidin- 4-yl)amino)thiazol-4-yl)- 2-((4- fluorophenoxy)methyl) pyrido[3,4-d]pyrimidin- 4(3H)-one | Scheme C | ¹H NMR (400 MHz, CD₃OD) δ 8.61 (d, 1H), 8.46 (s, 1H), 8.20 (d, 1H), 7.51 (m, 5H), 7.10 (m, 2H), 7.05 (m, 2H), 5.22 (s, 2H), 4.36 (s, 2H), 4.05 (m, 1H), 3.60 (d, 2H), 3.15 (t, 2H), 2.42 (d, 2H), 2.28 (br s, 1H), 1.86 (m, 2H) | 543 |
| 129 | 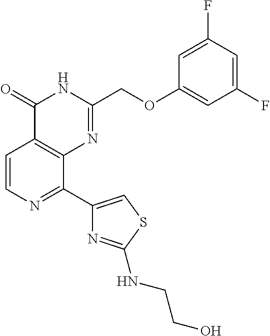 [SMILES: O=c1[nH]c(COc2cc(F)cc(F)c2)nc2c(-c3csc(NCCO)n3)nccc12] | 2-((3,5- difluorophenoxy)methyl)- 8-(2-((2- hydroxyethyl)amino) thiazol-4-yl)pyrido[3,4- d]pyrimidin-4(3H)-one | Scheme C | none | 431 |
| 130 | 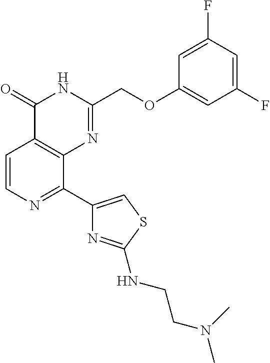 [SMILES: CN(C)CCNc1nc(-c2nccc3c(=O)[nH]c(COc4cc(F)cc(F)c4)nc23)cs1] | 2-((3,5- difluorophenoxy)methyl)- 8-(2-((2- (dimethylamino)ethyl) amino)thiazol-4- yl)pyrido[3,4-d]pyrimidin- 4(3H)-one | Scheme C | ¹H NMR (400 MHz, CD₃OD) δ 8.73 (d, 1H), 8.32 (s, 1H), 8.00 (d, 1H), 6.78 (dd, 2H), 6.60 (m, 1H), 5.24 (s, 2H), 3.74 (m, 2H), 3.40 (m, 2H), 3.01 (s, 6H) | 458 |
| 131 | 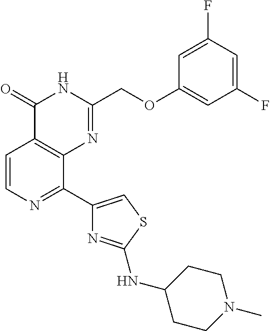 [SMILES: CN1CCC(Nc2nc(-c3nccc4c(=O)[nH]c(COc5cc(F)cc(F)c5)nc34)cs2)CC1] | 2-((3,5- difluorophenoxy)methyl)- 8-(2-((1-methylpiperidin- 4-yl)amino)thiazol-4- yl)pyrido[3,4-d]pyrimidin- 4(3H)-one | Scheme C | ¹H NMR (400 MHz, CD₃OD) δ 8.65 (d, 1H), 8.51 (s, 1H), 8.21 (d, 1H), 6.80 (dd, 2H), 6.62 (m, 1H), 5.28 (s, 2H), 3.64 (d, 2H), 3.16 (m, 2H), 2.92 (s, 3H), 2.44 (d, 2H), 2.27 (br s, 1H), 1.88 (m, 2H) | 485 |
| 132 | 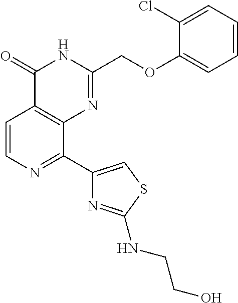 [SMILES: O=c1[nH]c(COc2ccccc2Cl)nc2c(-c3csc(NCCO)n3)nccc12] | 2-((2- chlorophenoxy)methyl)-8- (2-((2- hydroxyethyl)amino) thiazol-4-yl)pyrido[3,4- d]pyrimidin-4(3H)-one | Scheme C | ¹H NMR (400 MHz, CD₃OD) δ 8.64 (d, 1H), 8.27 (s, 1H), 8.07 (d, 1H), 7.44 (dd, 1H), 7.27 (m, 1H), 7.20 (dd, 1H), 7.01 (t, 1H), 5.28 (s, 2H), 3.81 (t, 2H), 3.60 (t, 2H) | 430 |
| 133 | 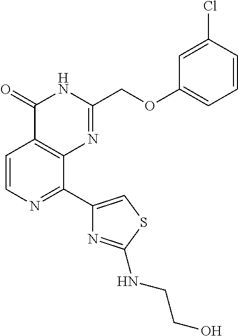 [SMILES: O=c1[nH]c(COc2cccc(Cl)c2)nc2c(-c3csc(NCCO)n3)nccc12] | 2-((3- chlorophenoxy)methyl)-8- (2-((2- hydroxyethyl)amino) thiazol-4-yl)pyrido[3,4- d]pyrimidin-4(3H)-one | Scheme C | ¹H NMR (400 MHz, CD₃OD) δ 8.65 (d, 1H), 8.20 (s, 1H), 8.06 (d, 1H), 7.30 (t, 1H), 7.18 (t, 1H), 7.03 (m, 2H), 5.24 (s, 2H), 3.83 (t, 2H), 3.62 (t, 2H) | 430 |
| 134 | 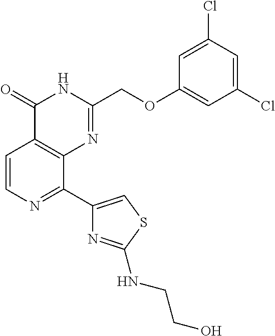 [SMILES: O=c1[nH]c(COc2cc(Cl)cc(Cl)c2)nc2c(-c3csc(NCCO)n3)nccc12] | 2-((3,5- dichlorophenoxy)methyl)- 8-(2-((2- hydroxyethyl)amino) thiazol-4-yl)pyrido[3,4- d]pyrimidin-4(3H)-one | Scheme C | ¹H NMR (400 MHz, CD₃OD) δ 8.68 (d, 1H), 8.31 (s, 1H), 8.10 (d, 1H), 7.16 (d, 2H), 7.10 (t, 1H), 5.26 (s, 2H), 3.84 (t, 2H), 3.63 (t, 2H) | 464 |
| 135 | 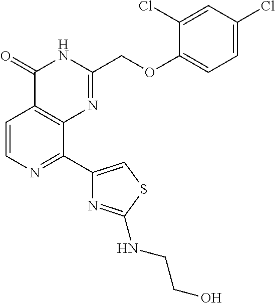 [SMILES: O=c1[nH]c(COc2ccc(Cl)cc2Cl)nc2c(-c3csc(NCCO)n3)nccc12] | 2-((2,4- dichlorophenoxy)methyl)- 8-(2-((2- hydroxyethyl)amino) thiazol-4-yl)pyrido[3,4- d]pyrimidin-4(3H)-one | Scheme C | ¹H NMR (400 MHz, CD₃OD) δ 8.67 (d, 1H), 8.31 (s, 1H), 8.10 (d, 1H), 7.51 (d, 1H), 7.28 (dd, 1H), 7.19 (d, 1H), 5.29 (s, 2H), 3.82 (t, 2H), 3.62 (t, 2H) | 464 |
| 136 | 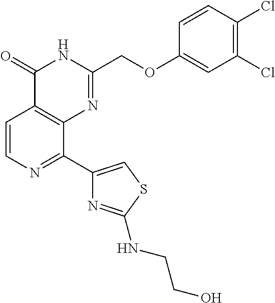 [SMILES: O=c1[nH]c(COc2ccc(Cl)c(Cl)c2)nc2c(-c3csc(NCCO)n3)nccc12] | 2-((3,4- dichlorophenoxy)methyl)- 8-(2-((2- hydroxyethyl)amino) thiazol-4-yl)pyrido[3,4- d]pyrimidin-4(3H)-one | Scheme C | ¹H NMR (400 MHz, CD₃OD) δ 8.68 (d, 1H), 8.28 (s, 1H), 8.11 (d, 1H), 7.46 (d, 1H), 7.37 (d, 1H), 7.10 (m, 1H), 5.26 (s, 2H), 3.84 (m, 2H), 3.64 (m, 2H) | 464 |
| 137 | 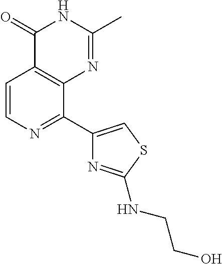 [SMILES: Cc1nc2c(-c3csc(NCCO)n3)nccc2c(=O)[nH]1] | 8-(2-((2- hydroxyethyl)amino) thiazol-4-yl)-2- methylpyrido [3,4- d]pyrimidin-4(3H)-one | Scheme E | ¹H NMR (400 MHz, CD₃OD) δ 8.63 (d, 1H), 8.42 (s, 1H), 8.06 (d, 1H), 3.66 (t, 2H), 3.64 (m, 2H), 2.59 (s, 3H) | 303 |
| 138 | 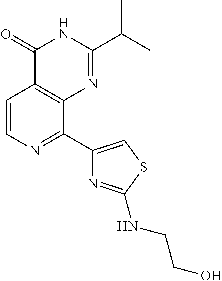 [SMILES: CC(C)c1nc2c(-c3csc(NCCO)n3)nccc2c(=O)[nH]1] | 8-(2-((2- hydroxyethyl)amino) thiazol-4-yl)-2- isopropylpyrido[3,4- d]pyrimidin-4(3H)-one | Scheme E | ¹H NMR (400 MHz, CD₃OD) δ 8.79 (d, 1H), 8.24 (d, 2H), 3.66 (m, 2H), 3.60 (m, 2H), 2.83 (s, 3H), 2.65 (m, 1H), 1.61 (s, 3H) | 331 |
| 139 | 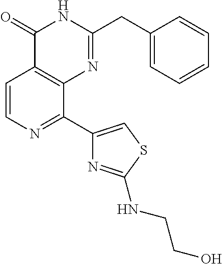 [SMILES: O=c1[nH]c(Cc2ccccc2)nc2c(-c3csc(NCCO)n3)nccc12] | 2-benzyl-8-(2-((2- hydroxyethyl)amino) thiazol-4-yl)pyrido[3,4- d]pyrimidin-4(3H)-one | Scheme D | ¹H NMR (400 MHz, CD₃OD) δ 8.60 (d, 1H), 8.06 (d, 1H), 7.88 (s, 1H), 7.41 (m, 5H), 4.16 (s, 2H), 3.83 (t, 2H), 3.59 (t, 2H) | 379 |
| 140 | 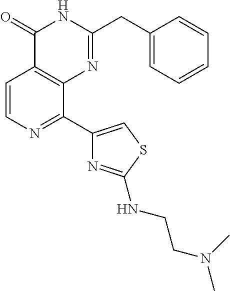 [SMILES: CN(C)CCNc1nc(-c2nccc3c(=O)[nH]c(Cc4ccccc4)nc23)cs1] | 2-benzyl-8-(2-((2- (dimethylamino)ethyl) amino)thiazol-4- yl)pyrido[3,4-d]pyrimidin- 4(3H)-one | Scheme D | ¹H NMR (400 MHz, CD₃OD) δ 8.66 (d, 1H), 7.95 (d, 1H), 7.76 (s, 2H), 7.40 (m, 5H), 4.13 (s, 2H), 3.69 (m, 2H), 3.37 (m, 2H), 2.99 (s, 3H) | 407 |
| 141 | 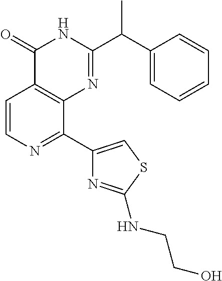 [SMILES: CC(c1ccccc1)c1nc2c(-c3csc(NCCO)n3)nccc2c(=O)[nH]1] | 8-(2-((2- hydroxyethyl)amino) thiazol-4-yl)-2-(1- phenylethyl)pyrido[3,4- d]pyrimidin-4(3H)-one | Scheme D | ¹H NMR (400 MHz, CD₃OD) δ 8.62 (d, 1H), 8.33 (s, 1H), 8.06 (d, 1H), 7.40 (d, 2H), 7.35 (t, 2H), 7.28 (t, 1H), 4.22 (q, 1H), 3.84 (t, 2H), 3.63 (t, 2H), 1.79 (d, 3H) | 393 |
| 142 | 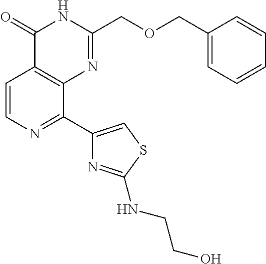 [SMILES: O=c1[nH]c(COCc2ccccc2)nc2c(-c3csc(NCCO)n3)nccc12] | 2-((benzyloxy)methyl)-8- (2-((2- hydroxyethyl)amino) thiazol-4-yl)pyrido[3,4- d]pyrimidin-4(3H)-one | Scheme D | none | 409 |
| 143 | 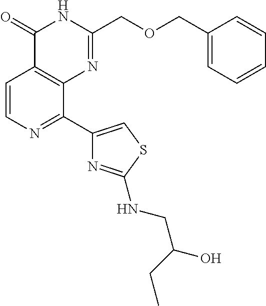 [SMILES: CCC(O)CNc1nc(-c2nccc3c(=O)[nH]c(COCc4ccccc4)nc23)cs1] | 2-((benzyloxy)methyl)-8- (2-((2- hydroxybutyl)amino) thiazol-4-yl)pyrido[3,4- d]pyrimidin-4(3H)-one | Scheme D | none | 438 |
| 144 | 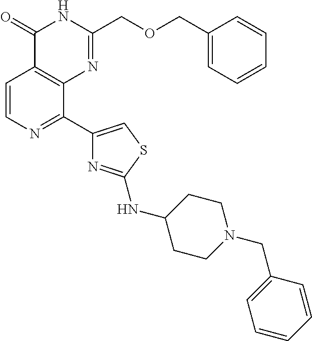 [SMILES: O=c1[nH]c(COCc2ccccc2)nc2c(-c3csc(NC4CCN(Cc5ccccc5)CC4)n3)nccc12] | 2-((benzyloxy)methyl)-8- (2-((1-benzylpiperidin-4- yl)amino)thiazol-4- yl)pyrido[3,4-d]pyrimidin- 4(3H)-one | Scheme D | ¹H NMR (400 MHz, CD₃OD) δ 8.61 (d, 1H), 8.57 (s, 1H), 8.18 (d, 1H), 7.53 (m, 6H), 7.33 (m, 4H), 4.71 (s, 1H), 4.67 (s, 2H), 4.36 (s, 2H), 4.31 (m, 2H), 3.57 (m, 2H), 3.11 (m, 2H), 2.34 (m, 2H), 2.18 (m, 1H), 1.84 (m, 2H) | 539 |
| 145 | 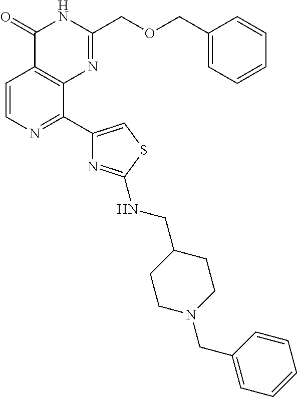 [SMILES: O=c1[nH]c(COCc2ccccc2)nc2c(-c3csc(NCC4CCN(Cc5ccccc5)CC4)n3)nccc12] | 2-((benzyloxy)methyl)-8- (2-(((1-benzylpiperidin-4- yl)methyl)amino)thiazol- 4-yl)pyrido[3,4- d]pyrimidin-4(3H)-one | Scheme D | ¹H NMR (400 MHz, CD₃OD) δ 8.64 (d, 1H), 8.40 (s, 1H), 8.09 (d, 1H), 7.50 (s, 4H), 7.39 (2H), 7.33 (m, 4H), 4.74 (s, 2H), 4.66 (s, 2H), 4.30 (s, 2H), 3.52 (d, 2H), 3.41 (m, 2H), 3.02 (t, 2H), 2.06 (d, 2H), 1.56 (t, 2H) | 553 |
| 146 | 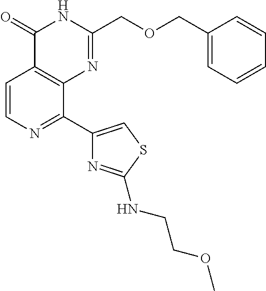 [SMILES: COCCNc1nc(-c2nccc3c(=O)[nH]c(COCc4ccccc4)nc23)cs1] | 2-((benzyloxy)methyl)-8- (2-((2- methoxyethyl)amino) thiazol-4-yl)pyrido[3,4- d]pyrimidin-4(3H)-one | Scheme D | ¹H NMR (400 MHz, CD₃OD) δ 8.66 (d, 1H), 8.39 (s, 1H), 8.08 (d, 1H), 7.41 (d, 2H), 7.33 (m, 3H), 4.74 (s, 2H), 4.64 (s, 2H), 3.67 (s, 4H), 3.43 (s, 3H) | 423 |
| 147 | 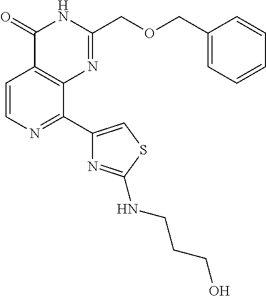 [SMILES: O=c1[nH]c(COCc2ccccc2)nc2c(-c3csc(NCCCO)n3)nccc12] | 2-((benzyloxy)methyl)-8- (2-((3- hydroxypropyl)amino) thiazol-4-yl)pyrido[3,4- d]pyrimidin-4(3H)-one | Scheme D | ¹H NMR (400 MHz, CD₃OD) δ 8.66 (d, 1H), 8.42 (s, 1H), 8.07 (d, 1H), 7.41 (d, 2H), 7.34 (m, 3H), 4.74 (s, 2H), 4.64 (s, 2H), 3.71 (t, 2H), 3.60 (t, 2H), 1.94 (m, 2H) | 423 |
| 148 | 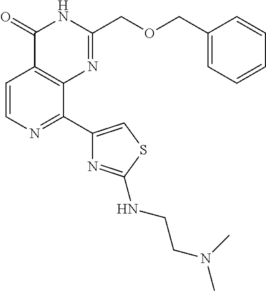 [SMILES: CN(C)CCNc1nc(-c2nccc3c(=O)[nH]c(COCc4ccccc4)nc23)cs1] | 2-((benzyloxy)methyl)-8- (2-((2- (dimethylamino)ethyl) amino)thiazol-4- yl)pyrido[3,4-d]pyrimidin- 4(3H)-one | Scheme D | ¹H NMR (400 MHz, CD₃OD) δ 8.65 (d, 1H), 8.62 (s, 1H), 8.06 (d, 1H), 7.41 (d, 2H), 7.33 (m, 3H), 4.74 (s, 2H), 4.64 (s, 2H), 3.79 (m, 2H), 3.44 (m, 2H), 3.00 (s, 6H) | 437 |
| 149 | 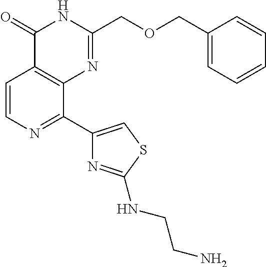 [SMILES: NCCNc1nc(-c2nccc3c(=O)[nH]c(COCc4ccccc4)nc23)cs1] | 8-(2-((2- aminoethyl)amino)thiazol- 4-yl)-2- ((benzyloxy)methyl)pyrido [3,4-d]pyrimidin-4(3H)- one | Scheme D | ¹H NMR (400 MHz, CD₃OD) δ 8.77 (s, 1H), 8.66 (d, 1H), 8.21 (d, 1H), 7.41 (d, 2H), 7.32 (m, 3H), 4.75 (s, 2H), 4.67 (s, 2H), 3.80 (t, 2H), 3.28 (m, 2H), | 408 |
| 150 | 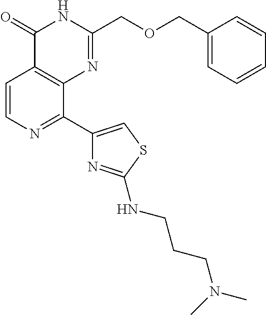 [SMILES: CN(C)CCCNc1nc(-c2nccc3c(=O)[nH]c(COCc4ccccc4)nc23)cs1] | 2-((benzyloxy)methyl)-8- (2-((3- (dimethylamino)propyl) amino)thiazol-4- yl)pyrido[3,4-d]pyrimidin- 4(3H)-one | Scheme D | ¹H NMR (400 MHz, CD₃OD) δ 8.66 (d, 1H), 8.56 (s, 1H), 8.10 (d, 1H), 7.36 (d, 2H), 7.32 (m, 3H), 4.74 (s, 2H), 4.66 (s, 2H), 3.61 (t, 2H), 3.24 (t, 2H), 2.91 (s, 6H), 2.14 (m, 2H) | 451 |
| 151 | 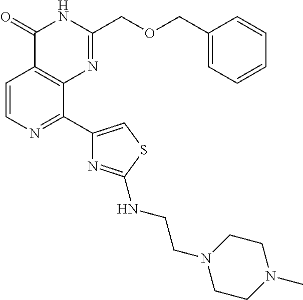 [SMILES: CN1CCN(CCNc2nc(-c3nccc4c(=O)[nH]c(COCc5ccccc5)nc34)cs2)CC1] | 2-((benzyloxy)methyl)-8- (2-((2-(4-methylpiperazin- 1-yl)ethyl)amino)thiazol- 4-yl)pyrido[3,4- d]pyrimidin-4(3H)-one | Scheme D | ¹H NMR (400 MHz, CD₃OD) δ 8.68 (d, 1H), 8.54 (s, 1H), 8.11 (d, 1H), 7.41 (d, 2H), 7.32 (m, 3H), 4.74 (s, 2H), 4.65 (s, 2H), 3.68 (t, 2H), 3.40 (m, 2H), 3.00 (t, 2H), 2.92 (3H) | 492 |
| 152 | 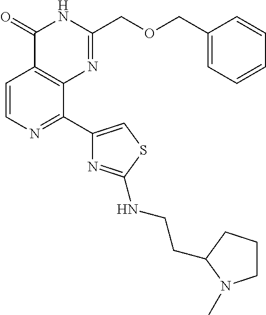 [SMILES: CN1CCCC1CCNc1nc(-c2nccc3c(=O)[nH]c(COCc4ccccc4)nc23)cs1] | 2-((benzyloxy)methyl)-8- (2-((2-(1- methylpyrrolidin-2- yl)ethyl)amino)thiazol-4- yl)pyrido[3,4-d]pyrimidin- 4(3H)-one | Scheme D | ¹H NMR (400 MHz, CD₃OD) δ 8.65 (s, 1H), 8.64 (d, 1H), 8.14 (d, 1H), 7.41 (d, 2H), 7.32 (m, 3H), 4.75 (s, 2H), 4.66 (s, 2H), 3.86 (t, 2H), 3.63 (m, 2H), 3.44 (t, 2H), 3.04 (m, 2H), 1.91 (m, 5H), 1.58 (m, 2H) | 477 |
| 153 | 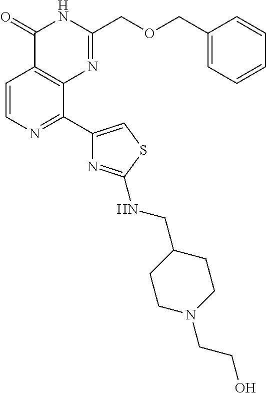 [SMILES: O=c1[nH]c(COCc2ccccc2)nc2c(-c3csc(NCC4CCN(CCO)CC4)n3)nccc12] | 2-((benzyloxy)methyl)-8- (2-(((1-(2- hydroxyethyl)piperidin-4- yl)methyl)amino)thiazol- 4-yl)pyrido[3,4- d]pyrimidin-4(3H)-one | Scheme D | ¹H NMR (400 MHz, CD₃OD) δ 8.65 (d, 1H), 8.51 (s, 1H), 8.12 (d, 1H), 7.41 (d, 2H), 7.32 (m, 3H), 4.74 (s, 2H), 4.66 (s, 2H), 3.88 (t, 2H), 3.68 (d, 2H), 3.45 (d, 2H), 3.23 (m, 2H), 3.04 (t, 2H), 2.08 (d, 2H), 1.82 (m, 1H), 1.65 (m, 2H) | 507 |
| 154 | 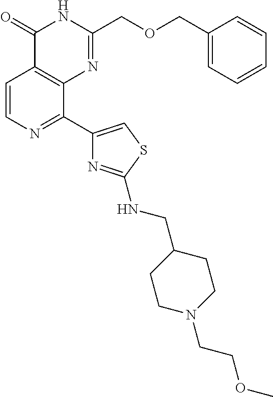 [SMILES: COCCN1CCC(CNc2nc(-c3nccc4c(=O)[nH]c(COCc5ccccc5)nc34)cs2)CC1] | 2-((benzyloxy)methyl)-8- (2-(((1-(2- methoxyethyl)piperidin-4- yl)methyl)amino)thiazol- 4-yl)pyrido[3,4- d]pyrimidin-4(3H)-one | Scheme D | ¹H NMR (400 MHz, CD₃OD) δ 8.64 (d, 1H), 8.51 (s, 1H), 8.11 (d, 1H), 7.41 (d, 2H), 7.32 (m, 3H), 4.74 (s, 2H), 4.66 (s, 2H), 3.70 (m, 2H), 3.65 (d, 2H), 3.44 (d, 2H), 3.40 (s, 3H), 3.31 (m, 3H), 3.02 (t, 2H), 2.08 (d, 2H), 1.64 (m, 2H) | 521 |
| 155 | 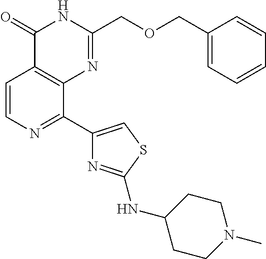 [SMILES: CN1CCC(Nc2nc(-c3nccc4c(=O)[nH]c(COCc5ccccc5)nc34)cs2)CC1] | 2-((benzyloxy)methyl)-8- (2-((1-methylpiperidin-4- yl)amino)thiazol-4- yl)pyrido[3,4-d]pyrimidin- 4(3H)-one | Scheme D | ¹H NMR (400 MHz, CD₃OD) δ 8.65 (d, 1H), 8.25 (s, 1H), 8.06 (d, 1H), 7.41 (d, 2H), 7.32 (m, 3H), 4.85 (s, 2H), 4.72 (s, 2H), 3.57 (d, 2H), 3.00 (m, 2H), 2.89 (s, 3H), 2.80 (m, 2H), 1.70 (m, 2H) | 463 |
| 156 | 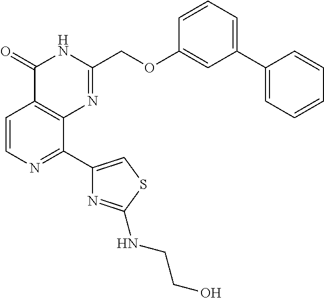 [SMILES: O=c1[nH]c(COc2cccc(-c3ccccc3)c2)nc2c(-c3csc(NCCO)n3)nccc12] | 2-(([1,1′-biphenyl]-3- yloxy)methyl)-8-(2-((2- hydroxyethyl)amino) thiazol-4-yl)pyrido[3,4- d]pyrimidin-4(3H)-one | Scheme C | ¹H NMR (600 MHz, CD₃OD) δ 8.66 (d, 1H), 8.19 (s, 1H), 8.09 (d, 1H), 7.58 (d, 3H), 7.39 (m, 5H), 7.33 (m, 1H), 5.32 (s, 2H), 3.81 (m, 2H), 3.59 (m, 2H) | 472 |
| 157 | 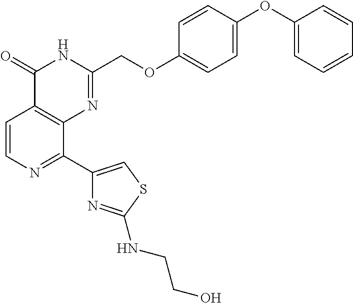 [SMILES: O=c1[nH]c(COc2ccc(Oc3ccccc3)cc2)nc2c(-c3csc(NCCO)n3)nccc12] | 8-(2-((2- hydroxyethyl)amino) thiazol-4-yl)-2-((4- phenoxyphenoxy)methyl) pyrido[3,4-d]pyrimidin- 4(3H)-one | Scheme C | ¹H NMR (600 MHz, CD₃OD) δ 8.66 (d, 1H), 8.28 (s, 1H), 8.09 (d, 1H), 7.27 (m, 2H), 7.11 (m, 2H), 7.04 (t, 1H), 6.96 (d, 2H), 6.89 (d, 2H), 5.22 (s, 2H), 3.83 (t, 2H), 3.63 (t, 2H), | 488 |
| 158 | 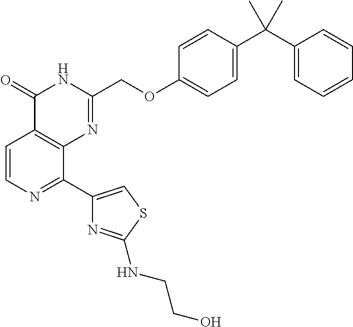 [SMILES: CC(C)(c1ccccc1)c1ccc(OCc2nc3c(-c4csc(NCCO)n4)nccc3c(=O)[nH]2)cc1] | 8-(2-((2- hydroxyethyl)amino) thiazol-4-yl)-2-((4-(2- phenylpropan-2- yl)phenoxy)methyl)pyrido [3,4-d]pyrimidin-4(3H)- one | Scheme C | ¹H NMR (600 MHz, CD₃OD) δ 8.65 (d, 1H), 8.22 (s, 1H), 8.08 (d, 1H), 7.19 (m, 6H), 7.11 (m, 1H), 6.99 (d, 2H), 5.20 (s, 2H), 3.81 (m, 2H), 3.61 (t, 2H), 1.63 (s, 6H) | 514 |
| 159 | 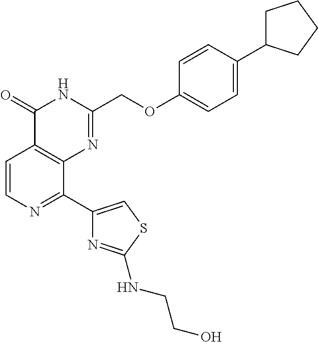 [SMILES: O=c1[nH]c(COc2ccc(C3CCCC3)cc2)nc2c(-c3csc(NCCO)n3)nccc12] | 2-((4- cyclopentylphenoxy) methyl)-8-(2-((2- hydroxyethyl)amino) thiazol-4-yl)pyrido[3,4- d]pyrimidin-4(3H)-one | Scheme C | ¹H NMR (600 MHz, CD₃OD) δ 8.65 (d, 1H), 8.14 (s, 1H), 8.09 (d, 1H), 7.19 (m, 2H), 7.00 (d, 2H), 5.21 (s, 2H), 3.82 (m, 2H), 3.61 (m, 2H), 2.95 (m, 1H), 2.01 (m, 2H), 1.79 (m, 2H), 1.67 (m, 2H), 1.54 (m, 2H) | 464 |
| 160 | 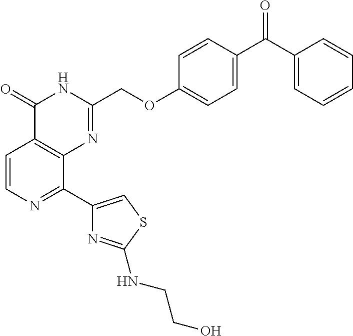 [SMILES: O=C(c1ccccc1)c1ccc(OCc2nc3c(-c4csc(NCCO)n4)nccc3c(=O)[nH]2)cc1] | 2-((4- benzoylphenoxy)methyl)- 8-(2-((2- hydroxyethyl)amino) thiazol-4-yl)pyrido[3,4- d]pyrimidin-4(3H)-one | Scheme C | ¹H NMR (600 MHz, CD₃OD) δ 8.67 (d, 1H), 8.18 (s, 1H), 8.09 (d, 1H), 7.82 (d, 2H), 7.71 (d, 2H), 7.62 (t, 1H), 7.51 (t, 2H), 7.25 (d, 2H), 5.26 (s, 2H), 3.82 (t, 2H), 3.61 (m, 2H) | 500 |
| 161 | 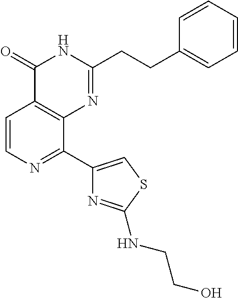 [SMILES: O=c1[nH]c(CCc2ccccc2)nc2c(-c3csc(NCCO)n3)nccc12] | 8-(2-((2- hydroxyethyl)amino) thiazol-4-yl)-2- phenethylpyrido[3,4- d]pyrimidin-4(3H)-one | Scheme D | ¹H NMR (400 MHz, CD₃OD) δ 8.61 (d, 1H), 8.16 (s, 1H), 8.04 (d, 1H), 7.27 (m, 4H), 7.16 (m, 1H), 3.83 (t, 2H), 3.60 (m, 2H), 3.20 (m, 2H), 3.12 (m, 2H) | 393 |
| 162 | 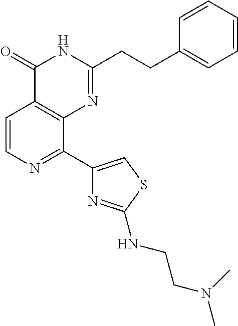 [SMILES: CN(C)CCNc1nc(-c2nccc3c(=O)[nH]c(CCc4ccccc4)nc23)cs1] | 8-(2-((2- (dimethylamino)ethyl) amino)thiazol-4-yl)-2- phenethylpyrido[3,4- d]pyrimidin-4(3H)-one | Scheme D | ¹H NMR (400 MHz, CD₃OD) δ 8.66 (d, 1H), 8.28 (s, 1H), 7.96 (d, 1H), 7.26 (m, 4H), 7.16 (m, 1H), 3.74 (m, 2H), 3.40 (m, 2H), 3.21 (m, 2H), 3.10 (m, 2H), 3.01 (s, 6H) | 421 |
| 163 | 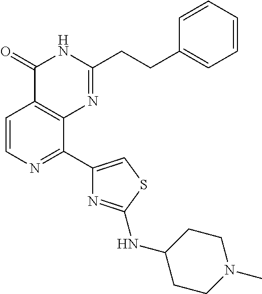 [SMILES: CN1CCC(Nc2nc(-c3nccc4c(=O)[nH]c(CCc5ccccc5)nc34)cs2)CC1] | 8-(2-((1-methylpiperidin- 4-yl)amino)thiazol-4-yl)- 2-phenethylpyrido[3,4- d]pyrimidin-4(3H)-one | Scheme D | ¹H NMR (400 MHz, CD₃OD) δ 8.57 (d, 1H), 8.40 (s, 1H), 8.17 (d, 1H), 7.28 (m, 4H), 7.19 (m, 1H), 4.10 (m, 2H), 3.61 (m, 2H), 3.16 (m, 4H), 2.92 (s, 3H), 2.44 (d, 2H), 2.20 (m, 1H), 1.82 (m, 2H) | 447 |
| 164 | 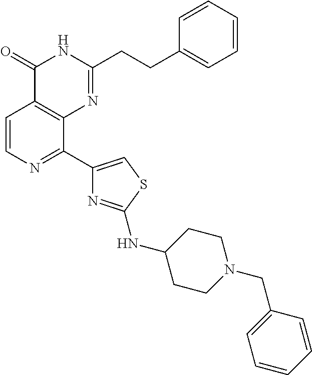 [SMILES: O=c1[nH]c(CCc2ccccc2)nc2c(-c3csc(NC4CCN(Cc5ccccc5)CC4)n3)nccc12] | 8-(2-((1-benzylpiperidin- 4-yl)amino)thiazol-4-yl)- 2-phenethylpyrido[3,4- d]pyrimidin-4(3H)-one | Scheme D | none | 523 |
| 165 | 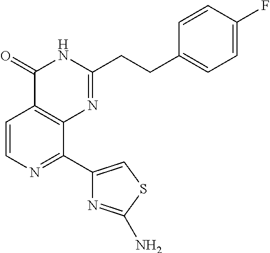 [SMILES: Nc1nc(-c2nccc3c(=O)[nH]c(CCc4ccc(F)cc4)nc23)cs1] | 8-(2-aminothiazol-4-yl)-2- (4- fluorophenethyl)pyrido[3, 4-d]pyrimidin-4(3H)-one | Scheme D | ¹H NMR (600 MHz, CD₃OD) δ 1.95-2.13 (m, 2 H), 2.22- 2.37 (m, 2 H), 3.31-3.40 (m, 2H), 7.11-7.21 (m, 2 H), 7.23-7.34 (m, 3 H), 7.51-7.67 (m, 1 H), 7.69-7.77 (m, 1 H), 10.41 (s, 1 H) | 368 |
| 166 | 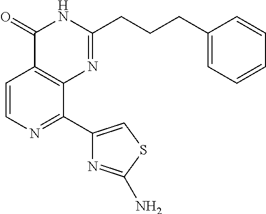 [SMILES: Nc1nc(-c2nccc3c(=O)[nH]c(CCCc4ccccc4)nc23)cs1] | 8-(2-aminothiazol-4-yl)-2- (3- phenylpropyl)pyrido[3,4- d]pyrimidin-4(3H)-one | Scheme D | ¹H NMR (600 MHz, CD₃OD) δ 0.82-0.97 (m, 4 H), 2.55- 2.74 (m, 2 H), 3.66-3.82 (m, 2 H), 7.10-7.27 (m, 1 H), 7.39-7.50 (m, 1 H), 7.50-7.66 (m, 1 H), 7.70 (m, 1 H), 7.92- 8.04 (m, 1 H), 8.04-8.16 (m, 1 H), 8.21 (s, 1 H) 8.36 (m, 1 H) 8.53-8.70 (m, 1 H) 10.45 (s, 1 H) | 364 |
| 167 | 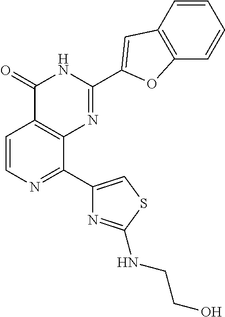 [SMILES: O=c1[nH]c(-c2cc3ccccc3o2)nc2c(-c3csc(NCCO)n3)nccc12] | 2-(benzofuran-2-yl)-8-(2- ((2- hydroxyethyl)amino) thiazol-4-yl)pyrido[3,4- d]pyrimidin-4(3H)-one | Scheme D | ¹H NMR (400 MHz, CD₃OD) δ 8.59 (s, 1H) 7.95-8.05 (m, 2H), 7.74 (d, J = 7.83 Hz, 1H), 7.67 (d, J = 7.83 Hz, 1H), 7.42-7.59 (m, 2H), 7.33- 7.38 (m, 1H), 3.83-3.89 (m, 2H), 3.67 (t, J = 4.89 Hz, 2H) | 406 |
| 168 | 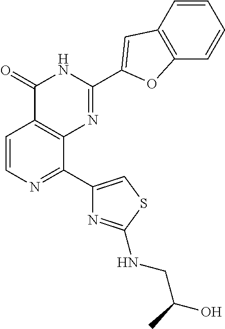 [SMILES: C[C@H](O)CNc1nc(-c2nccc3c(=O)[nH]c(-c4cc5ccccc5o4)nc23)cs1] | (S)-2-(benzofuran-2-yl)-8- (2-((2- hydroxypropyl)amino) thiazol-4-yl)pyrido[3,4- d]pyrimidin-4(3H)-one | Scheme D | ¹H NMR (400 MHz, CD₃OD) δ 8.63 (s, 1H), 8.58-8.61 (d, 1H), 8.03 (d, J = 5.09 Hz, 1H), 7.97 (s, 1H), 7.74 (d, J = 7.83 Hz, 1H), 7.66-7.70 (m, 1H), 7.42-7.52 (m, 1H), 7.31- 7.39 (m, 1H), 3.60 (dd, J = 13.69, 3.13 Hz, 2H), 3.44 (dd, J = 13.89, 7.24 Hz, 1H), 1.28 (d, J = 6.26 Hz, 3H) | 420 |
| 169 | 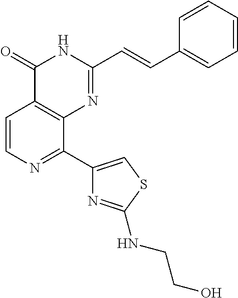 [SMILES: O=c1[nH]c(/C=C/c2ccccc2)nc2c(-c3csc(NCCO)n3)nccc12] | (E)-8-(2-((2- hydroxyethyl)amino) thiazol-4-yl)-2- styrylpyrido[3,4- d]pyrimidin-4(3H)-one | Scheme D | ¹H NMR (600 MHz, CD₃OD) δ 8.78-8.83 (m, 1H), 8.12- 8.28 (m, 1H), 7.58-7.70 (m, 2H), 7.28-7.45 (m, 4H), 4.60 (br d, J = 5.28 Hz, 1H), 3.12 (t, J = 5.87 Hz, 2H), 3.31- 3.40 (m, 2H), 2.53-2.64 (m, 1H) | 391 |
| 170 | 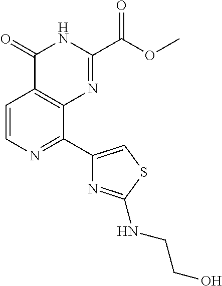 [SMILES: COC(=O)c1nc2c(-c3csc(NCCO)n3)nccc2c(=O)[nH]1] | methyl 8-(2-((2- hydroxyethyl)amino) thiazol-4-yl)-4-oxo-3,4- dihydropyrido[3,4- d]pyrimidine-2- carboxylate | Scheme D | ¹H NMR (600 MHz, CD₃OD) δ 8.62-8.75 (m, 1H), 8.41 (s, 1H), 7.98-8.13 (m, 1H), 4.63 (br d, J = 5.28 Hz, 1H), 3.49 (t, J = 5.87 Hz, 2H), 3.31- 3.40 (m, 1H), 3.14-3.27 (s, 3H) | 347 |
| 171 | 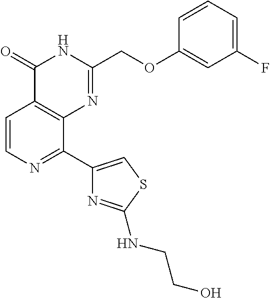 [SMILES: O=c1[nH]c(COc2cccc(F)c2)nc2c(-c3csc(NCCO)n3)nccc12] | 2-((3- fluorophenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.66 (d, 1H), 8.24 (s, 1H), 8.08 (d, 1H), 7.31 (m, 1H), 6.92 (m, 2H), 6.76 (m, 1H), 5.24 (s, 2H), 3.82 (t, 2H), 3.62 (t, 2H) | 413 |
| 172 | 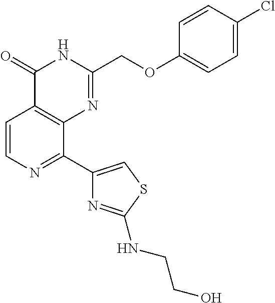 [SMILES: O=c1[nH]c(COc2ccc(Cl)cc2)nc2c(-c3csc(NCCO)n3)nccc12] | 2-((4- chlorophenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.66 (d, 1H), 8.22 (s, 1H), 8.09 (d, 1H), 7.30 (d, 2H), 7.09 (d, 2H), 5.22 (s, 2H), 3.83 (t, 2H), 3.62 (t, 2H) | 430 |
| 173 | 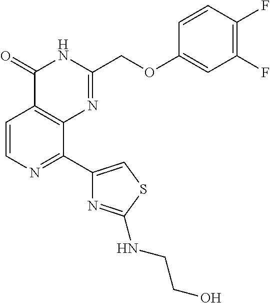 [SMILES: O=c1[nH]c(COc2ccc(F)c(F)c2)nc2c(-c3csc(NCCO)n3)nccc12] | 2-((3,4- difluorophenoxy)methyl)- 8-(2-((2- hydroxyethyl)amino) thiazol-4-yl)pyrido[3,4- d]pyrimidin-4(3H)-one | Scheme C | ¹H NMR (600 MHz, CD₃OD) δ 8.67 (d, 1H), 8.33 (s, 1H), 8.09 (d, 1H), 7.21 (q, 1H), 7.10 (m, 1H), 6.90 (m, 1H), 5.21 (s, 2H), 3.83 (t, 2H), 3.63 (t, 2H) | 431 |
| 174 | 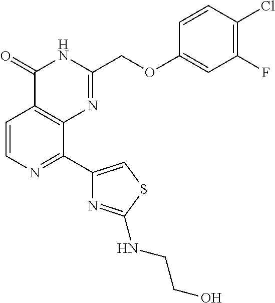 [SMILES: O=c1[nH]c(COc2ccc(Cl)c(F)c2)nc2c(-c3csc(NCCO)n3)nccc12] | 2-((4-chloro-3- fluorophenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.66 (d, 1H), 8.26 (s, 1H), 8.07 (d, 1H), 7.40 (t, 1H), 7.08 (dd, 1H), 6.96 (m, 1H), 5.23 (s, 2H), 3.83 (t, 2H), 3.62 (t, 2H) | 448 |
| 175 | 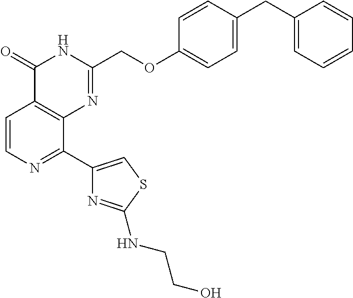 [SMILES: O=c1[nH]c(COc2ccc(Cc3ccccc3)cc2)nc2c(-c3csc(NCCO)n3)nccc12] | 2-((4- benzylphenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.65 (d, 1H), 8.12 (s, 1H), 8.08 (d, 1H), 7.22 (t, 2H), 7.15 (m, 5H), 7.02 (d, 2H), 5.21 (s, 2H), 3.90 (s, 2H), 3.81 (t, 2H), 3.60 (t, 2H) | 486 |
| 176 | 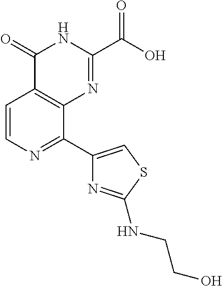 [SMILES: O=C(O)c1nc2c(-c3csc(NCCO)n3)nccc2c(=O)[nH]1] | 8-(2-((2- hydroxyethyl)amino) thiazol-4-yl)-4-oxo-3,4- dihydropyrido[3,4- d]pyrimidine-2-carboxylic acid | Scheme D | ¹H NMR (600 MHz, CD₃OD) δ 8.76-8.82 (m, 1 H), 8.40- 8.51 (s, 1 H), 7.96-8.10 (m, 1 H), 4.65 (br d, J = 5.28 Hz, 1 H), 3.55 (m, 2 H), 3.31-3.40 (m, 2 H) | 333 |
| 177 | 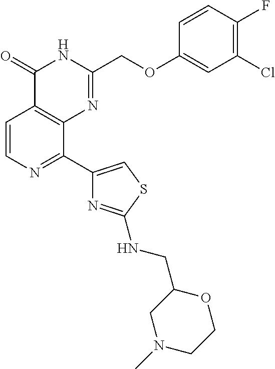 [SMILES: CN1CCOC(CNc2nc(-c3nccc4c(=O)[nH]c(COc5ccc(F)c(Cl)c5)nc34)cs2)C1] | 2-((3-chloro-4- fluorophenoxy)methyl)-8- (2-(((4-methylmorpholin- 2- yl)methyl)amino)thiazol- 4-yl)pyrido[3,4- d]pyrimidin-4(3H)-one | Scheme C | ¹H NMR (600 MHz, CD₃OD) δ 8.64 (d, 1H), 8.36 (s, 1H), 8.13 (d, 1H), 7.28 (m, 1H), 7.19 (t, 1H), 7.07 (m, 1H), 5.22 (s, 2H), 4.17 (dd, 1H), 4.06 (br, 1H), 3.85 (t, 1H), 3.78 (d, 1H), 3.67 (m, 1H), 3.58 (br, 1H), 3.45 (br, 1H), 3.13 (br, 1H), 3.00 (br, 1H), 2.31 (s, 3H) | 517 |
| 178 | 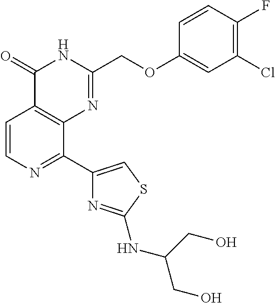 [SMILES: O=c1[nH]c(COc2ccc(F)c(Cl)c2)nc2c(-c3csc(NC(CO)CO)n3)nccc12] | 2-((3-chloro-4- fluorophenoxy)methyl)-8- (2-((1,3-dihydroxypropan- 2-yl)amino)thiazol-4- yl)pyrido[3,4-d]pyrimidin- 4(3H)-one | Scheme C | ¹H NMR (600 MHz, CD₃OD) δ 8.66 (d, 1H), 8.27 (s, 1H), 8.08 (d, 1H), 7.28 (m, 1H), 7.19 (t, 1H), 7.06 (m, 1H), 5.22 (s, 2H), 3.84-3.75 (m, 5H) | 478 |
| 179 | 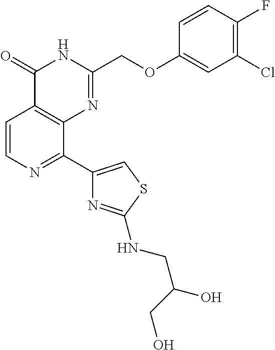 [SMILES: O=c1[nH]c(COc2ccc(F)c(Cl)c2)nc2c(-c3csc(NCC(O)CO)n3)nccc12] | 2-((3-chloro-4- fluorophenoxy)methyl)-8- (2-((2,3- dihydroxypropyl)amino) thiazol-4-yl)pyrido[3,4- d]pyrimidin-4(3H)-one | Scheme C | ¹H NMR (600 MHz, CD₃OD) δ 8.67 (d, 1H), 8.30 (s, 1H), 8.09 (d, 1H), 7.28 (m, 1H), 7.19 (t, 1H), 7.07 (m, 1H), 5.22 (s, 2H), 3.92 (m, 1H), 3.70-3.53 (m, 4H) | 478 |
| 180 | 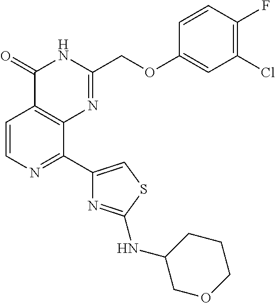 [SMILES: O=c1[nH]c(COc2ccc(F)c(Cl)c2)nc2c(-c3csc(NC4CCCOC4)n3)nccc12] | 2-((3-chloro-4- fluorophenoxy)methyl)-8- (2-((tetrahydro-2H-pyran- 3-yl)amino)thiazol-4- yl)pyrido[3,4-d]pyrimidin- 4(3H)-one | Scheme C | ¹H NMR (600 MHz, CD₃OD) δ 8.65 (d, 1H), 8.34 (s, 1H), 8.13 (d, 1H), 7.28 (m, 1H), 7.20 (t, 1H), 7.07 (m, 1H), 5.22 (s, 2H), 3.94 (dd, 1H), 3.88 (m, 1H), 3.74 (m, 1H), 3.67 (m, 1H), 3.57 (m, 1H), 2.10 (m, 1H), 1.84 (m, 2H), 1.66 (m, 1H) | 488 |
| 181 | 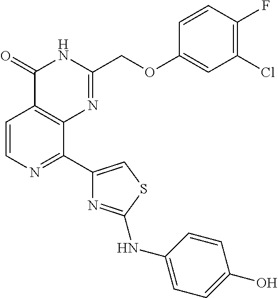 [SMILES: O=c1[nH]c(COc2ccc(F)c(Cl)c2)nc2c(-c3csc(Nc4ccc(O)cc4)n3)nccc12] | 2-((3-chloro-4- fluorophenoxy)methyl)-8- (2-((4- hydroxyphenyl)amino) thiazol-4-yl)pyrido[3,4- d]pyrimidin-4(3H)-one | Scheme C | ¹H NMR (600 MHz, CD₃OD) δ 8.68 (d, 1H), 8.33 (s, 1H), 8.13 (d, 1H), 7.34 (d, 2H), 7.23 (m, 1H), 7.18 (t, 1H), 7.02 (m, 1H), 6.88 (d, 2H), 5.15 (s, 2H), | 496 |
| 182 | 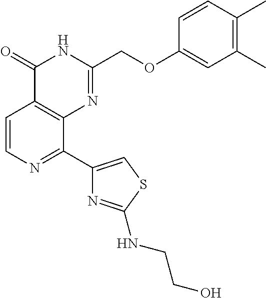 [SMILES: Cc1ccc(OCc2nc3c(-c4csc(NCCO)n4)nccc3c(=O)[nH]2)cc1C] | 2-((3,4- dimethylphenoxy)methyl)- 8-(2-((2- hydroxyethyl)amino) thiazol-4-yl)pyrido[3,4- d]pyrimidin-4(3H)-one | Scheme C | ¹H NMR (600 MHz, CD₃OD) δ 8.64 (d, 1H), 7.97 (s, 1H), 7.05 (d, 1H), 6.92 (s, 1H), 6.33 (d, 2H), 5.15 (s, 2H), 3.55 (m, 2H), 3.40 (m, 2H), 2.23 (s, 3H), 2.19 (s, 3H) | 423 |
| 183 | 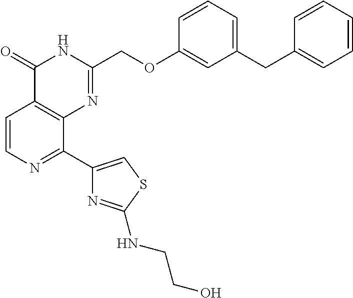 [SMILES: O=c1[nH]c(COc2cccc(Cc3ccccc3)c2)nc2c(-c3csc(NCCO)n3)nccc12] | 2-((3- benzylphenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.66 (d, 1H), 8.06 (d, 1H), 7.88 (s, 1H), 7.24 (t, 1H), 7.05-6.88 (m, 8H), 5.20 (s, 2H), 3.89 (s, 2H), 3.78 (t, 2H), 3.57 (t, 2H) | 486 |
| 184 | 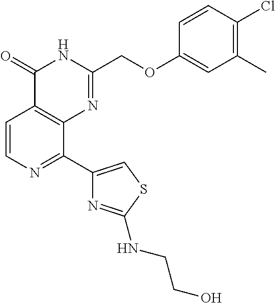 [SMILES: Cc1cc(OCc2nc3c(-c4csc(NCCO)n4)nccc3c(=O)[nH]2)ccc1Cl] | 2-((4-chloro-3- methylphenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.65 (d, 1H), 8.04 (d, 2H), 7.27 (d, 1H), 7.08 (s, 1H), 6.94 (d, 1H), 5.20 (s, 2H), 3.76 (m, 2H), 3.58 (m, 2H), 2.33 (s, 3H) | 444 |
| 185 | 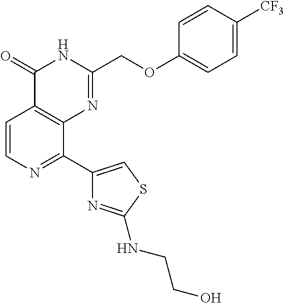 [SMILES: O=c1[nH]c(COc2ccc(C(F)(F)F)cc2)nc2c(-c3csc(NCCO)n3)nccc12] | 8-(2-((2- hydroxyethyl)amino) thiazol-4-yl)-2-((4- (trifluoromethyl)phenoxy) methyl)pyrido[3,4- d]pyrimidin-4(3H)-one | Scheme C | ¹H NMR (600 MHz, CD₃OD) δ 8.67 (d, 1H), 8.22 (s, 1H), 8.10 (d, 1H), 7.63 (d, 2H), 7.26 (d, 2H), 5.32 (s, 2H), 3.83 (m, 2H), 3.62 (m, 2H) | 463 |
| 186 | 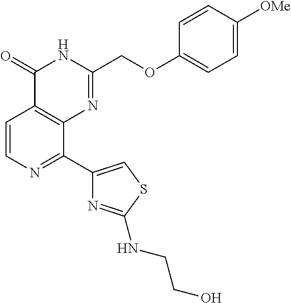 [SMILES: COc1ccc(OCc2nc3c(-c4csc(NCCO)n4)nccc3c(=O)[nH]2)cc1] | 8-(2-((2- hydroxyethyl)amino) thiazol-4-yl)-2-((4- methoxyphenoxy)methyl) pyrido[3,4-d]pyrimidin- 4(3H)-one | Scheme C | ¹H NMR (600 MHz, CD₃OD) δ 8.65 (d, 1H), 8.04 (d, 2H), 7.04 (d, 2H), 6.87 (d, 2H), 5.16 (s, 2H), 3.74 (s, 3H), 3.58 (m, 2H), 3.33 (m, 2H) | 425 |
| 187 | 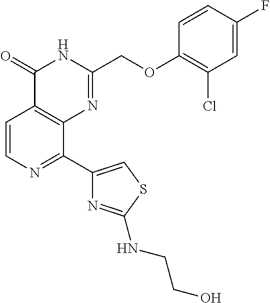 [SMILES: O=c1[nH]c(COc2ccc(F)cc2Cl)nc2c(-c3csc(NCCO)n3)nccc12] | 2-((2-chloro-4- fluorophenoxy)methyl)-8- (2-((2- hydroxyethyl)amino) thiazol-4-yl)pyrido[3,4- d]pyrimidin-4(3H)-one | Scheme C | ¹H NMR (600 MHz, CD₃OD) δ 8.67 (d, 1H), 8.30 (s, 1H), 8.08 (d, 1H), 7.28 (m, 1H), 7.22 (m, 1H), 7.05 (m, 1H), 5.25 (s, 2H), 3.79 (t, 2H), 3.60 (t, 2H) | 448 |
| 188 | 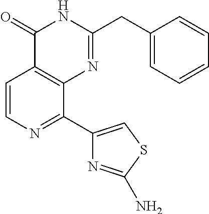 [SMILES: Nc1nc(-c2nccc3c(=O)[nH]c(Cc4ccccc4)nc23)cs1] | 8-(2-aminothiazol-4-yl)-2- benzylpyrido[3,4- d]pyrimidin-4(3H)-one | Scheme D | none | 335 |
| 189 | 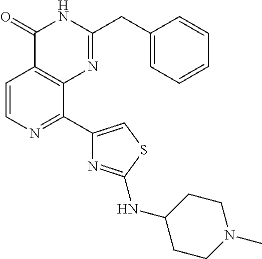 [SMILES: CN1CCC(Nc2nc(-c3nccc4c(=O)[nH]c(Cc5ccccc5)nc34)cs2)CC1] | 2-benzyl-8-(2-((1- methylpiperidin-4- yl)amino)thiazol-4- yl)pyrido[3,4-d]pyrimidin- 4(3H)-one | Scheme D | none | 433 |
| 190 | 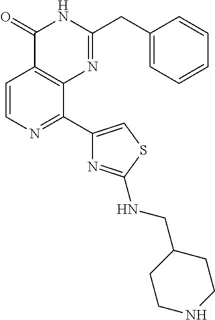 [SMILES: O=c1[nH]c(Cc2ccccc2)nc2c(-c3csc(NCC4CCNCC4)n3)nccc12] | 2-benzyl-8-(2-((piperidin- 4-ylmethyl)amino)thiazol- 4-yl)pyrido[3,4- d]pyrimidin-4(3H)-one | Scheme D | none | 433 |

Meanwhile, the compound represented by the formula (I) may have an asymmetric carbon center, and if having the asymmetric carbon center, may exist as an optical isomer, a diastereomer or a racemate, and all forms of isomers including these may be also within the scope of the compound according to one embodiment of the present invention.

Further, a pharmaceutically acceptable salt of the compound represented by the formula (I), or a pharmaceutically acceptable salt of the isomers of the compound represented by the formula (I) may be also within the scope of the compound of the above described one embodiment. For example, non-limiting examples of the pharmaceutically acceptable salt of the compound represented by the formula (I) or the isomer thereof may include a salt with an inorganic acid such as hydrochloric acid, hydrobromic acid, phosphoric acid or sulfuric acid; a salt with an organic carboxylic acid such as acetic acid, trifluoroacetic acid, citric acid, maleic acid, oxalic acid, succinic acid, benzoic acid, tartaric acid, fumaric acid, mandelic acid, ascorbic acid or malic acid, or a salt with a sulfonic acid such as methane sulfonic acid or p-toluene sulfonic acid; a salt with an alkali metal such as sodium, potassium or lithium; a salt with various acids known to be capable of forming other pharmaceutically acceptable salts, or the like.

The compound of the above formula (I) exhibits excellent effects on modulating catalytic activity of Histone Lysine Demethylase (KDMs), thereby having an outstanding potential for a pharmaceutical intervention of various cancer and any other diseases related to KDM dysregulation.

Further, another embodiment of the present invention provides a pharmaceutical composition including the above compound, the isomer thereof or the pharmaceutically acceptable salt thereof as an effective ingredient. More preferably, the pharmaceutical composition may be used for treatment or prevention of various cancer and disease related to KDM dysregulation. In certain embodiments, the disease may be a hyperproliferative disease, cancer, stroke, diabetes, hepatomegaly, cardiovascular disease, multiple sclerosis, Alzheimer's disease, cystic fibrosis, viral disease, autoimmune diseases, atherosclerosis, restenosis, psoriasis, rheumatoid arthritis, inflammatory bowel disease, asthma, allergic disorders, inflammation, neurological disorders, a hormone-related disease, conditions associated with organ transplantation, immunodeficiency disorders, destructive bone disorders, infectious disease, pathologic immune conditions involving T cell activation, CNS disorders or a myeloproliferative disorder. More preferably, the cancer may be selected from the group consisting of embryonic carcinoma, teratoma, seminoma, germ cell tumors, prostate cancer, breast cancer, stomach cancer, gastrointestinal cancer, neuroblastoma, choriocarcinoma, yolk sac tumors, ovarian cancer, endometrial cancer, cervical cancer, retinoblastoma, kidney cancer, liver cancer, gastric cancer, brain cancer, medulloblastoma, medulloepithelioma, glioma, glioblastoma, multiple myeloma, lung cancer, bronchial cancer, mesothelioma, skin cancer, colon and rectal cancer, bladder cancer, pancreatic cancer, lip and oral cancer, laryngeal and pharyngeal cancer, melanoma, pituitary cancer, penile cancer, parathyroid cancer, thyroid cancer, pheochromocytoma and paraganglioma, thymoma and thymic carcinoma, leukemia, lymphoma, plasma cell neoplasms, myeloproliferative disorders, islet cell tumor, small intestine cancer, transitional cell cancer, pleuropulmonary blastoma, gestational trophoblastic cancer, esophageal cancer, central nervous system cancer, head and neck cancer, endocrine cancer, cardiovascular cancer, rhabdomyosarcoma, soft tissue carcinomas, carcinomas of bone, cartilage, fat, vascular, neural, and hematopoietic tissues and AIDS-related cancers.

A pharmaceutical composition including the compound of the formula (I), the isomer thereof or the pharmaceutically acceptable salt thereof, as an effective ingredient may be used in the form of a general medicinal preparation. The medicinal preparation may be administered in form of various formulations such as oral and parenteral formulation, and the kind of said formulation may be variously determined depending on usage.

The inventive pharmaceutical composition may be formulated in accordance with any of the conventional methods in the form of various oral formulation such as tablet, granule, powder, capsule, syrup, emulsion or microemulsion, or parenteral administration including intramuscular, intravenous and subcutaneous routes.

If the composition is formulated into various oral and parenteral formulations, it may be prepared using a generally used excipient such as a filler, a diluent, a bulking agent, a binder, a wetting agent, a disintergrating agent, and a surfactant.

A solid preparation for oral administration may include tablets, pills, powders, granules, capsules, and the like, and the solid preparation may be prepared by mixing the compound represented by the formula (I), the isomer thereof, or the pharmaceutically acceptable salt thereof with at least one excipient, for example, starch, calcium carbonate, sucrose or lactose, gelatin, and the like. Further, in addition to a simple excipient, a lubricant such as magnesium stearate and talc may be used.

Further, a liquid preparation for oral administration may be suspensions, oral liquids, emulsions, syrups, and the like, and include various excipients, for example, a wetting agent, a sweetener, an aromatic, a preservative, and the like, in addition to water and liquid paraffin which are a simple diluent to be commonly used.

The preparation for parenteral administration includes a sterile aqueous solution, a non-aqueous solvent, a suspension, an emulsion, a freeze-dried preparation, a suppository and the like. As the non-aqueous solvent and the suspension solvent, propylene glycol, polyethylene glycol, a vegetable oil such as an olive oil, injectable ester such as ethyl oleate, and the like may be used. As a base of the suppository, witepsol, microgol, tween 61, cacao butter, laurin butter, glycerogelatin, and the like may be used.

Further, the effective amount of the compound of the formula (I), the isomer thereof or a pharmaceutically acceptable salt thereof used in the pharmaceutical composition may range about 0.1 to about 1,000 mg. A dosage or dose may be administered in various dosages and methods, for example, in divided dosages from once to several times a day depending on a patient's weight, age, sex, a health condition, diet, administration time, an administration method, an excretion rate, and severity of a disease.

The inventive compound of formula (I), the isomer thereof, or a pharmaceutically acceptable salt thereof may be administered orally or parenterally as an active ingredient in an effective amount ranging from about 0.1 to 1,000 mg, preferably 1 to 5,000 mg per a day in case of mammals including human in a single to 4 divided doses per a day, or on/off schedules. The dosage of the active ingredient may be adjusted in light of various relevant factors such as the condition of the subject to be treated (weight, age, sex, a health condition, diet), type and seriousness of illness, administration rate, administration time, administration method (route), an excretion rate and opinion of doctor. In certain cases, an amount less than the above dosage may be suitable. An amount greater than the above dosage may be used unless it causes deleterious side effects and such amount can be administered in divided doses per day.

Further, the present invention provides a method for preventing or treating various cancers or diseases related to KDM dysregulation, which comprises administering the inventive compound to a mammal in need thereof.

In a preferred embodiment, the compound of formula (I) may be prepared by various processes illustrated in Schemes A to G which are shown in the below examples.

### EXAMPLES

The present invention is further described and illustrated in more detail with reference to the examples provided below. These examples, however, should not be interpreted as limiting the scope of the present disclosure in any manner.

### 1. Chemical Synthetic Route

Several general approaches to the compounds of the present invention are illustrated as below. In Schemes A to G, R¹ and R² have the same meaning as defined above. All compounds of the present invention may be prepared via Schemes A to G.

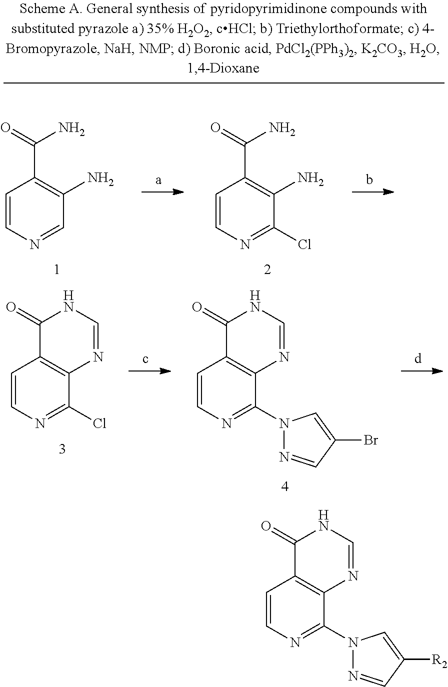

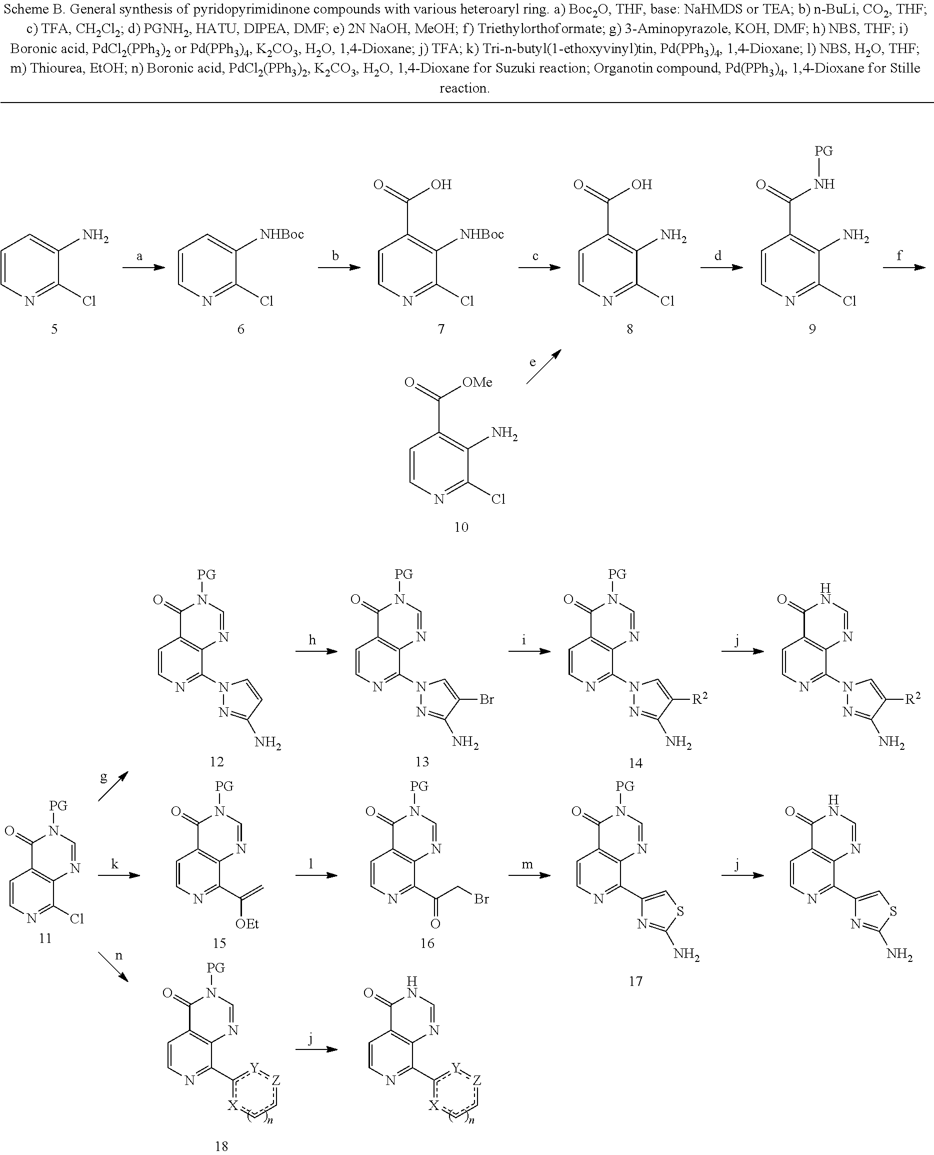

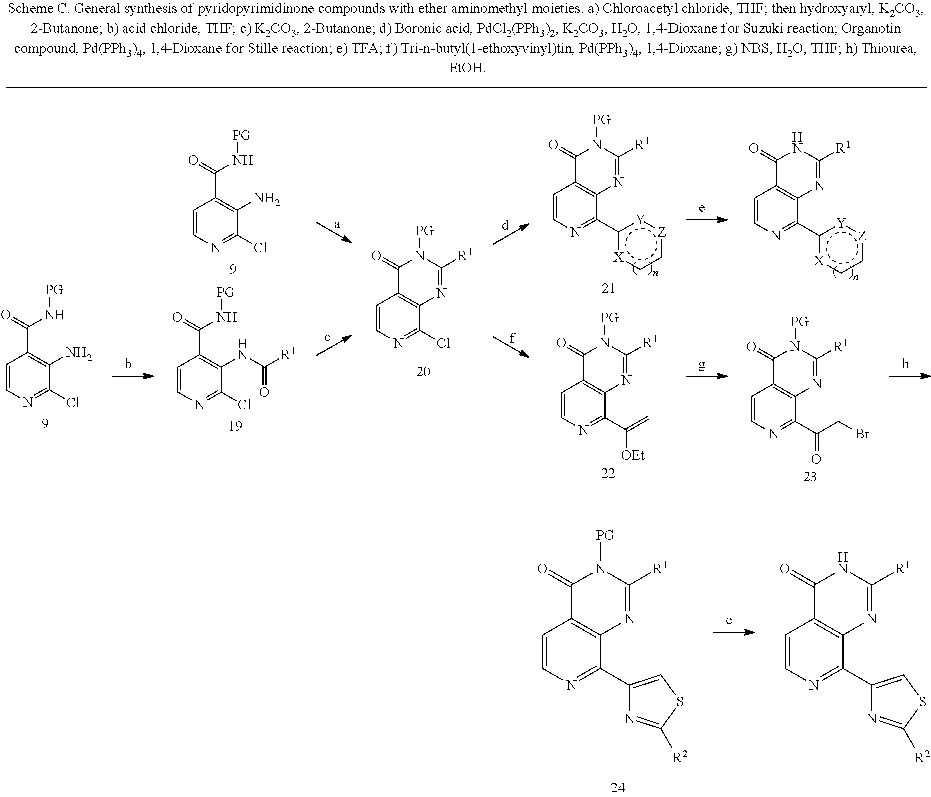

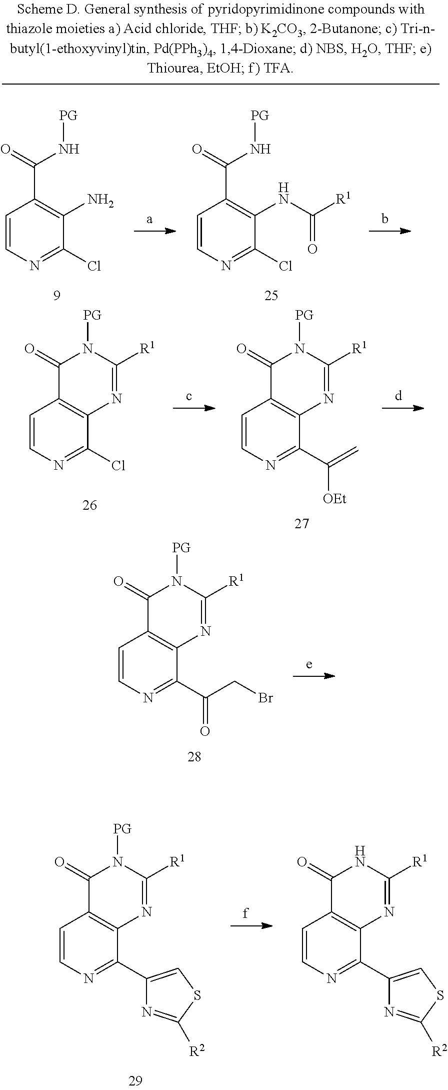

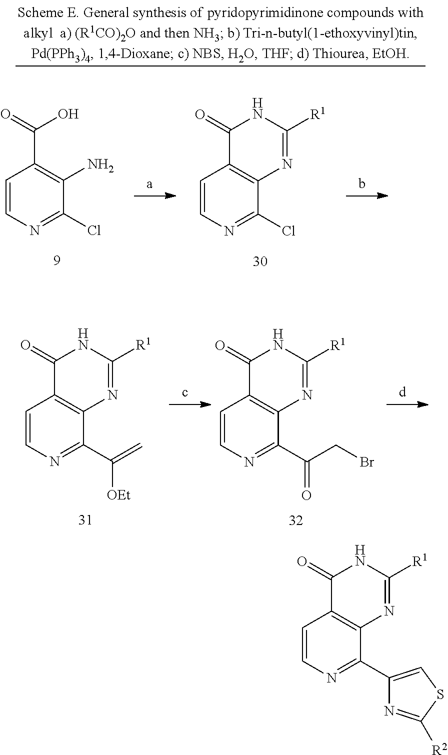

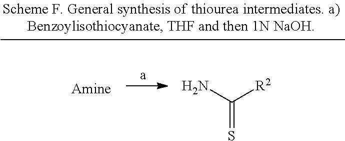

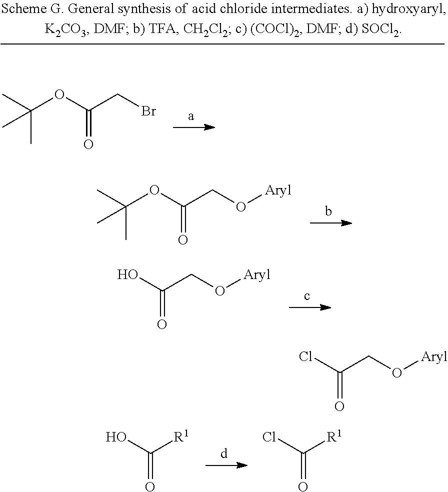

### 2. Chemical Synthesis

The representative compounds were prepared via the general synthetic routes (scheme A to G) described below in example 1-8.

### Example 1

### 8-(4-(m-tolyl)-1H-pyrazol-1-yl)pyrido[3,4-d]pyrimidin-4(3H)-one

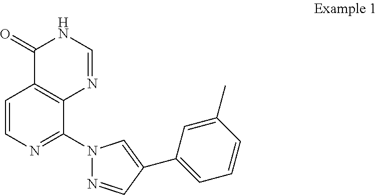 [SMILES: Cc1cccc(-c2cnn(-c3nccc4c(=O)[nH]cnc34)c2)c1]

Scheme for the preparation of the Compound of Example 1:

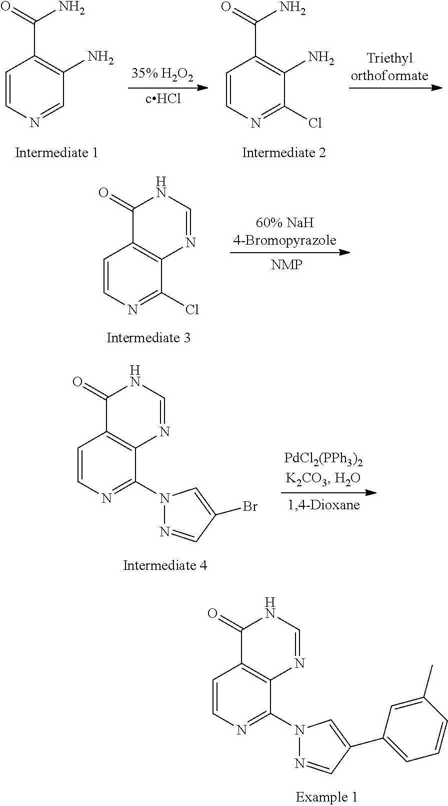 [SMILES: NC(=O)c1ccncc1N ; NC(=O)c1ccnc(Cl)c1N ; O=c1[nH]cnc2c(Cl)nccc12 ; O=c1[nH]cnc2c(-n3cc(Br)cn3)nccc12 ; Cc1cccc(-c2cnn(-c3nccc4c(=O)[nH]cnc34)c2)c1]

Intermediate 2.

An 3-aminoisonicotinamide (1 eq) was dissolved in concentrated HCl at 20° C. H₂O₂ (35%, 1.1 eq) was added dropwise during 30 minutes and the reaction mixture was stirred for another 30 minutes. The mixture was neutralized with sodium hydroxide solution to pH 8 and extracted with dichloromethane. The combined organic layer was dried over MgSO₄, filtered and concentrated in vacuo. The concentrated residue was purified by flash column chromatography.

Intermediate 3.

The intermediate 2 (1.0 mmol) was dissolved in triethylorthoformate (4 mL). The mixture was heated at 150° C. and allowed to stir for 1 day. The solvent was concentrated in vacuo to afford the Intermediate 3 (quant.).

Intermediate 4.

To a solution of 4-bromopyrazole in NMP was added 60% NaH and intermediate 3. The reaction mixture was heated to 180° C. and allowed to stir for 2 hours. After being cooled to room temperature, the mixture was concentrated in vacuo. The concentrated residue was purified by flash column chromatography to afford the Intermediate 4.

### Compound of Example 1

To a solution of Intermediate 4 (36 mg, 0.123 mmol) in 1,4-dioxane (4 ml) and water (2 ml) were added m-tolylboronic acid (20 mg, 0.147 mmol), [1,1′-Bis(diphenylphosphino)ferrocene] dichloropalladium(II) dichloromethane adduct (9 mg, 0.0123 mmol) and potassium carbonate (51 mg, 0.369 mmol). The mixture was heated to 140° C. for 30 minutes in a microwave. The mixture was then diluted with water (30 mL) and extracted with ethyl acetate (40 mL). The separated organic layer was dried over MgSO₄, filtered and concentrated in vacuo. The concentrated residue was purified by flash column chromatography to afford the Example 1.

### Example 2

### 8-(3-amino-1′-methyl-1H,1′H-[4,4′-bipyrazol]-1-yl)pyrido[3,4-d]pyrimidin-4(3H)-one

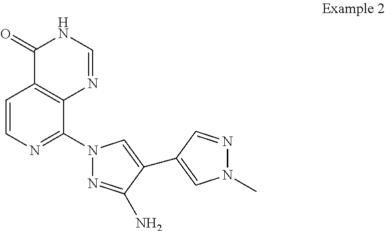 [SMILES: Cn1cc(-c2cn(-c3nccc4c(=O)[nH]cnc34)nc2N)cn1]

Scheme for the preparation of the Compound of Example 2:

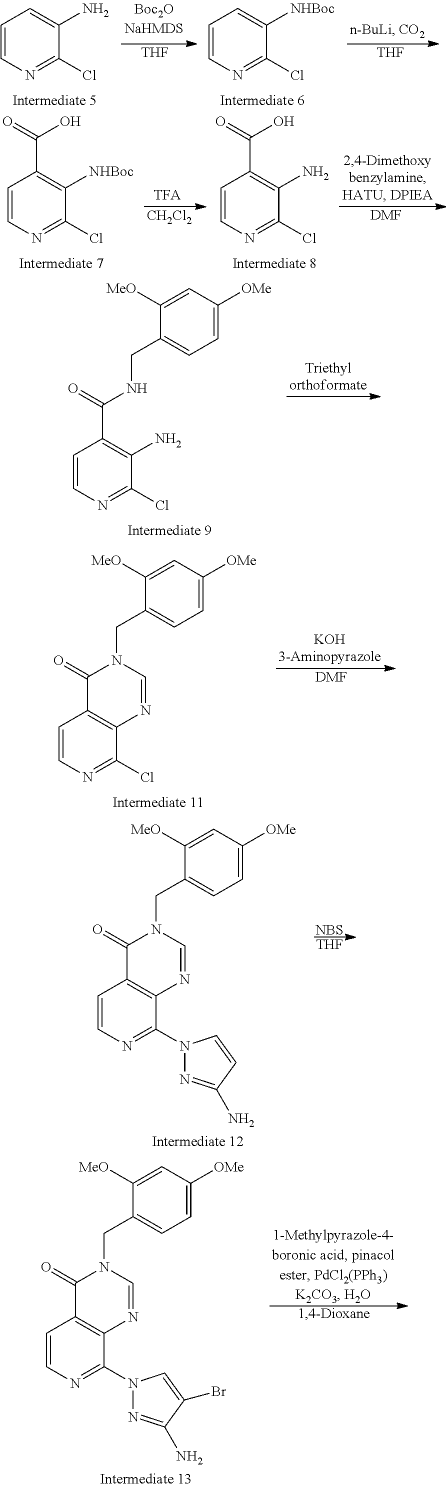

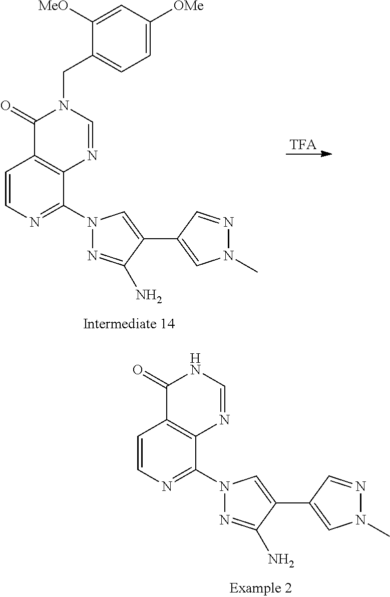 [SMILES: COc1ccc(Cn2cnc3c(-n4cc(-c5cnn(C)c5)c(N)n4)nccc3c2=O)c(OC)c1 ; Cn1cc(-c2cn(-c3nccc4c(=O)[nH]cnc34)nc2N)cn1 ; "TFA"]

Intermediate 6.

To a solution of 3-amino-2-chloropyridine (intermediate 5; 5 g, 38.9 mmol) in THF (35 ml) was added 2 M NaHMDS in THF (38.9 ml, 77.8 mmol). After being allowed to stir at room temperature for 15 minutes, Boc₂O (7.7 g, 35.6 mmol) in THF (20 ml) was added in one portion and then allowed to stir for 5 h at room temperature. 0.1% aqueous HCl was added and the mixture was extracted with EtOAc. The separated organic layer was dried over MgSO₄, filtered and concentrated in vacuo. The concentrated residue was purified through silica gel eluted with 0-50% EtOAc in hexane to afford the Intermediate 6 (7.18 g, 89%).

¹H NMR (300 MHz, CDCl₃) δ 8.52 (d, 1H), 8.05 (dd, 1H), 7.23 (dd, 1H), 7.02 (br s, 1H), 1.55 (s, 9H)

MS (ESI+) m/z 229 (M+H)⁺

Intermediate 7.

To a solution of Intermediate 6 (1.996 g, 8.728 mmol) in THF was slowly added 2.5 M n-BuLi in THF (10.4 ml). The mixture was allowed to stir for 30 min at −50° C. and dry-ice was added in several portions. After being allowed to stir for 30 min, the reaction was cooled to room temperature and extracted with EtOAc. The aqueous layer was acidified with AcOH and extracted with EtOAc. The separated organic layer was dried over MgSO₄, filtered and concentrated in vacuo to afford Intermediate 7 (1.418 g, 60%).

MS (ESI+) m/z 273 (M+H)⁺

Intermediate 8.

To a solution of intermediate 7 in CH₂Cl₂ was added trifluoroacetic acid at room temperature.

After being allowed to stir for overnight at room temperature, the mixture was concentrated in vacuo to afford Intermediate 8 as TFA salt.

MS (ESI+) m/z 173 (M+H)⁺

Intermediate 9.

To a solution of Intermediate 8 (476 mg, 2.76 mmol) and 2,4-dimethoxybenzylamine (0.46 mL, 3.06 mmol) in N,N-dimethylformamide (5 mL) was added diisopropylethylamine (0.97 mL, 5.54 mmol) and HATU (1.26 g, 3.32 mmol) at room temperature. The reaction mixture was allowed to stir for 30 min, concentrated in vacuo, diluted with EtOAc and washed with brine. The separated organic layer was dried over MgSO₄, filtered and concentrated in vacuo. The concentrated residue was purified by flash column chromatography to afford the Intermediate 9 (797 mg, 2.479 mmol) as a pale yellow oil.

¹H NMR (400 MHz, DMSO-d₆) δ 8.95 (m, 1H), 7.60 (d, J=5.2 Hz, 1H), 7.49 (d, J=4.8 Hz, 1H), 7.06 (d, J=8.4 Hz, 1H), 6.52 (d, J=2.4 Hz, 1H), 6.45 (dd, J=2.4, 8.4 Hz, 1H), 4.31 (d, J=6 Hz, 2H), 3.76 (s, 3H), 3.70 (s, 3H)

MS (ESI+) m/z 322 (M+H)⁺

Intermediate 11.

The intermediate 9 (322 mg, 1.0 mmol) was dissolved in triethylorthoformate (4 mL). The mixture was heated at 150° C. and allowed to stir for 4 days. The solvent was concentrated in vacuo to afford the Intermediate 10 (quant.) as a white solid.

¹H NMR (400 MHz, DMSO-d₆) δ 8.61 (s, 1H), 8.41 (d, J=5.2 Hz, 1H), 7.92 (d, J=5.2 Hz, 1H), 7.19 (d, J=8.8 Hz, 1H), 6.53 (d, J=2.4 Hz, 1H), 6.44 (dd, J=2.4, 8.8 Hz, 1H), 5.00 (s, 2H), 3.77 (s, 3H), 3.70 (s, 3H)

MS (ESI+) m/z 332 (M+H)⁺

Intermediate 12.

To solution of Intermediate 10 (350 mg, 1.054 mmol) and potassium hydroxide (118 mg, 2.108 mmol) in N,N-dimethylformamide was added 3-aminopyrazole (105 mg, 1.266 mmol) at room temperature. The reaction mixture was heated to 180° C. for 30 minutes in a microwave. The mixture was then diluted with water (30 mL) and extracted with ethyl acetate (40 mL). The separated organic layer was dried over MgSO₄, filtered and concentrated in vauo. The concentrated residue was purified by flash column chromatography to afford the Intermediate 11 (401 mg, qunt.).

Intermediate 13.

To a solution of Intermediate 11 (401 mg, 1.058 mmol) in tetrahydrofuran was added N-bromosuccinimide (207 mg, 1.164 mmol) at room temperature. The reaction mixture was allowed to stir for overnight. The mixture was then diluted with water and extracted with ethyl acetate. The separated organic layer was dried over MgSO₄, filtered and concentrated in vauo. The concentrated residue was purified by flash column chromatography to afford the Intermediate 12 (343 mg, 0.750 mmol).

Intermediate 14.

To a solution of Intermediate 12 (120 mg, 0.262 mmol) in 1,4-dioxane (4 ml) and water (2 ml) were added 1-Methylpyrazole-4-boronic acid, pinacol ester (60 mg, 0.288 mmol), [1,1′-Bis(diphenylphosphino)ferrocene] dichloropalladium(II) dichloromethane adduct (18 mg, 0.0262 mmol) and potassium carbonate (109 mg, 0.786 mmol). The mixture was heated to 140° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 13.

### Compound of Example 2

The Intermediate 13 was dissolved in TFA (2 ml). The mixture was allowed to stir for 1 h at 50° C. After being cooled to room temperature, the mixture was concentrated in vacuo. The concentrated residue was purified by preparative HPLC to afford the compound of Example 2.

### Example 3

### 8-(2-((2-hydroxyethyl)amino)thiazol-4-yl)pyrido[3,4-d]pyrimidin-4(3H)-one

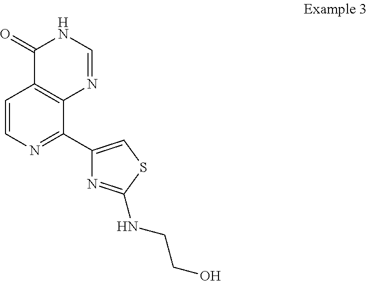 [SMILES: O=c1[nH]cnc2c(-c3csc(NCCO)n3)nccc12]

Scheme for the preparation of the Compound of Example 3:

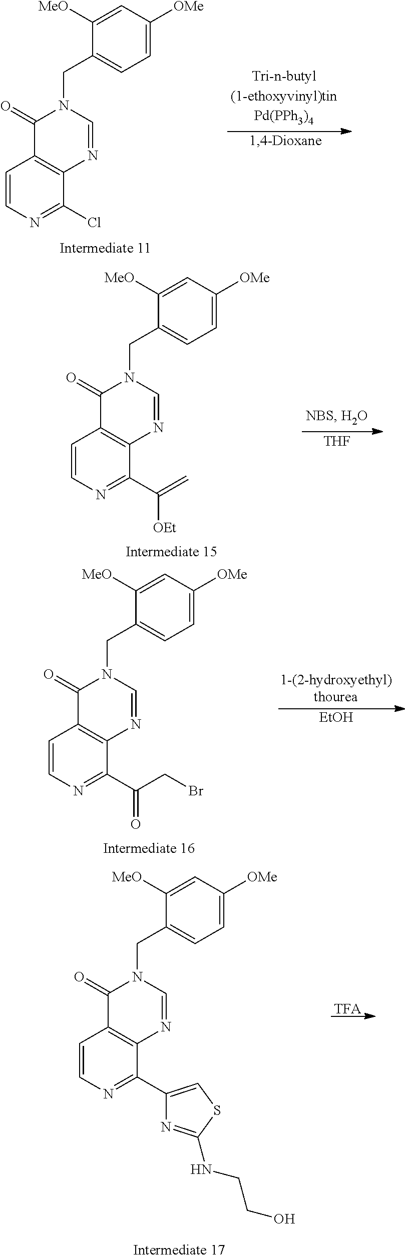

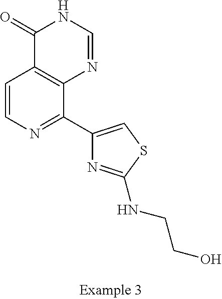 [SMILES: O=c1[nH]cnc2c(-c3csc(NCCO)n3)nccc12]

Intermediate 15.

To a solution of Intermediate 11 (100 mg, 0.301 mmol) in 1,4-dioxane was added Tri-n-butyl(1-ethoxyvinyl)tin (218 mg, 0.602 mmol) and Tetrakis(triphenylphosphine)palladium(0) (35 mg, 0.0301 mmol). The mixture was heated to 140° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 15.

Intermediate 16.

To a solution of Intermediate 15 (106 mg, 0.288 mmol) in tetrahydrofuran (5 ml) and water (1 ml) was added N-bromosuccinimide (46 mg, 0.259 mmol) at room temperature. The reaction mixture was allowed to stir for overnight. The mixture was then diluted with water and extracted with ethyl acetate. The separated organic layer was dried over MgSO₄, filtered and concentrated in vauo. The concentrated residue was purified by flash column chromatography to afford the Intermediate 16 (74 mg, 0.201 mmol).

Intermediate 17.

To a solution of Intermediate 16 (17 mg, 0.029 mmol) in ethanol was added 1-(2-hydroxyethyl)thiourea (6 mg, 0.044 mmol). The reaction mixture was heated at reflux for overnight. After being cooled to room temperature, the mixture was concentrated in vacuo. The concentrated residue was purified by preparative HPLC to afford the Intermediate 17 (11 mg, 0.019 mmol).

### Compound of Example 3

The Intermediate 17 was dissolved in TFA (2 ml). The mixture was allowed to stir for 1 h at 50° C. After being cooled to room temperature, the mixture was concentrated in vacuo. The concentrated residue was purified by preparative HPLC to afford the compound of Example 3.

### Example 4

### 8-(1-(2-hydroxyethyl)-1H-pyrazol-4-yl)pyrido[3,4-d]pyrimidin-4(3H)-one

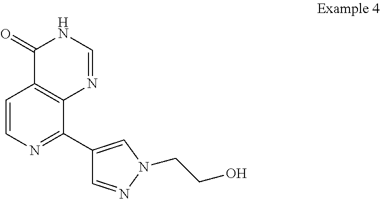 [SMILES: O=c1[nH]cnc2c(-c3cnn(CCO)c3)nccc12]

Scheme for the preparation of the Compound of Example 4:

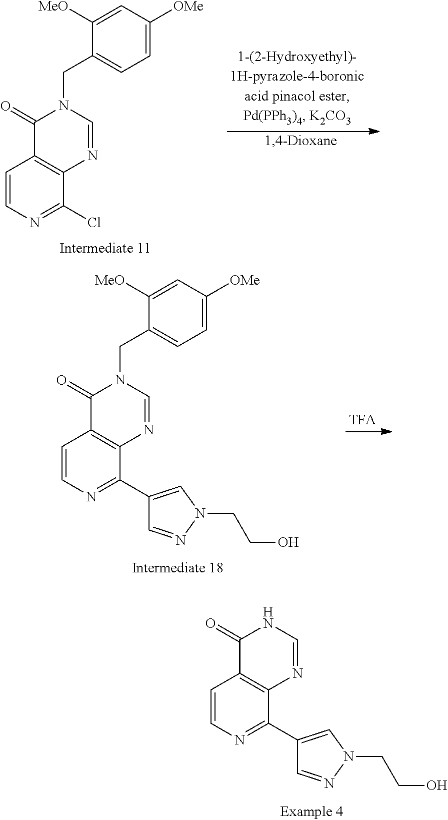 [SMILES: COc1ccc(Cn2cnc3c(Cl)nccc3c2=O)c(OC)c1 ; "1,4-Dioxane" ; COc1ccc(Cn2cnc3c(-c4cnn(CCO)c4)nccc3c2=O)c(OC)c1 ; O=c1[nH]cnc2c(-c3cnn(CCO)c3)nccc12]

Intermediate 18.

To a solution of Intermediate 11 (68 mg, 0.153 mmol) in 1,4-dioxane (4 ml) and water (2 ml) were added 1-(2-hydroxy)-1H-pyrazole-4-boronic acid pinacol ester (0.184 mmol), tetrakis(triphenylphosphine)palladium(o) (18 mg, 0.0153 mmol) and potassium carbonate (64 mg, 0.459 mmol). The mixture was heated to 140° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 18.

### Compound of Example 4

The Intermediate 18 was dissolved in TFA (2 ml). The mixture was allowed to stir for 1 h at room temperature and then concentrated in vacuo. The concentrated residue was purified by preparative HPLC to afford the compound of Example 4.

### Example 5

### 2-((4-benzylphenoxy)methyl)-8-(thiazol-2-yl)pyrido[3,4-d]pyrimidin-4(3H)-one

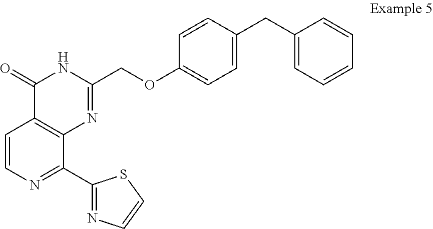 [SMILES: O=c1[nH]c(COc2ccc(Cc3ccccc3)cc2)nc2c(-c3nccs3)nccc12]

Scheme for the preparation of the Compound of Example 5:

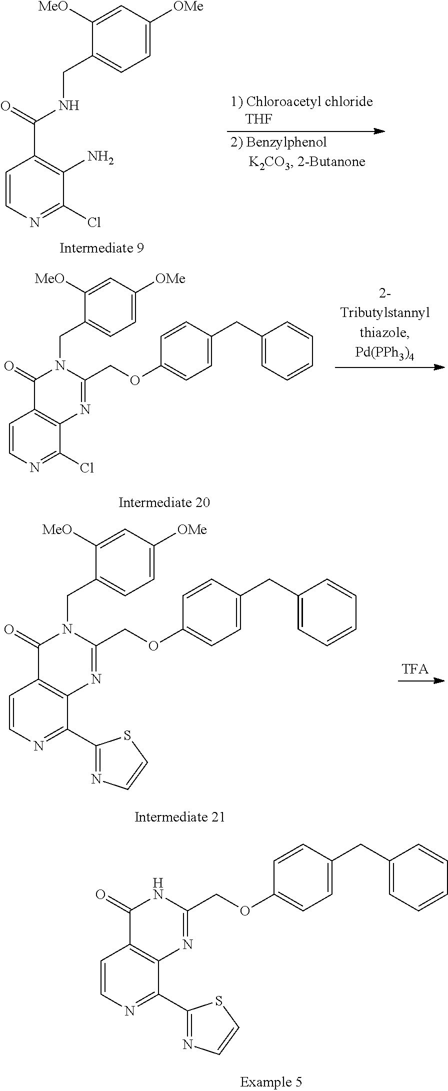 [SMILES: COc1ccc(CNC(=O)c2ccnc(Cl)c2N)c(OC)c1 ; COc1ccc(Cn2c(COc3ccc(Cc4ccccc4)cc3)nc3c(Cl)nccc3c2=O)c(OC)c1 ; COc1ccc(Cn2c(COc3ccc(Cc4ccccc4)cc3)nc3c(-c4nccs4)nccc3c2=O)c(OC)c1 ; O=c1[nH]c(COc2ccc(Cc3ccccc3)cc2)nc2c(-c3nccs3)nccc12]

Intermediate 20.

To a solution of Intermediate 9 (644 mg, 2.0 mmol) in THF (20 ml) was added chloroacetyl chloride (477 ul, 6.0 mmol) at room temperature. The mixture was allowed to stir for 2 h, concentrated to dryness in vacuo and then the resulting residue was dissolved in 2-butanone (40 ml). 4-Benzylphenol (442 mg, 2.4 mmol) and K₂CO₃ (828 mg, 6.0 mmol) were added to the residue, and then heated at reflux for overnight. After being cooled to room temperature, the mixture was extracted with EtOAc (50 ml). The separated organic layer was dried over MgSO₄, filtered and concentrated in vacuo. The concentrated residue was purified by flash column chromatography to afford the Intermediate 20 (517 mg, 0.98 mmol) ¹H NMR (400 MHz, CDCl₃) 8.42 (d, J=5.2 Hz, 1H), 8.00 (d, J=5.2 Hz, 1H), 7.26 (m, 2H), 7.15 (m, 3H), 7.10 (d, J=8.4 Hz, 2H), 6.98 (d, J=8.8 Hz, 1H), 6.88 (d, J=8.4 Hz, 2H), 6.37 (m, 2H), 5.43 (s, 2H), 5.16 (s, 2H), 3.91 (m, 2H), 3.74 (s, 3H), 3.58 (s, 3H)

MS (ESI+) m/z 528 (M+H)⁺

Intermediate 21.

To a solution of Intermediate 20 (105 mg, 0.200 mmol) in 1,4-dioxane was added 2-tributylstannyl thiazole (126 ul, 0.400 mmol) and Tetrakis(triphenylphosphine)palladium(0) (23 mg, 0.020 mmol) at room temperature. The mixture was heated to 140° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 21 (86 mg, 0.150 mmol).

### Compound of Example 5

The Intermediate 21 was dissolved in TFA (2 ml). The mixture was allowed to stir for 1 h at room temperature and then concentrated in vacuo. The concentrated residue was purified by preparative HPLC to afford the compound of Example 5.

### Example 6

### 2-((4-benzylphenoxy)methyl)-8-(2-(phenylamino)thiazol-4-yl)pyrido[3,4-d]pyrimidin-4(3H)-one

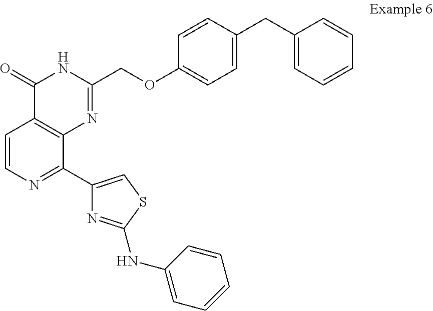 [SMILES: O=c1[nH]c(COc2ccc(Cc3ccccc3)cc2)nc2c(-c3csc(Nc4ccccc4)n3)nccc12]

Scheme for the preparation of the Compound of Example 6:

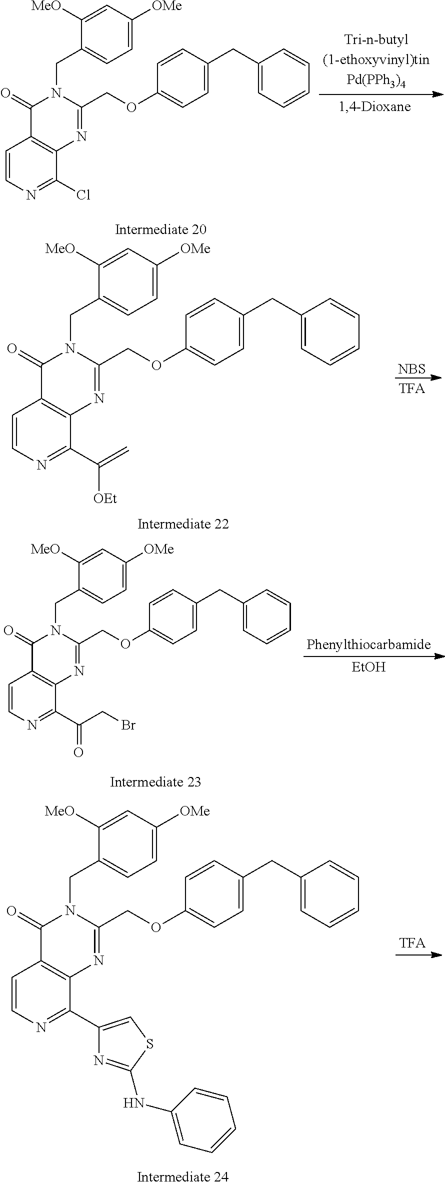

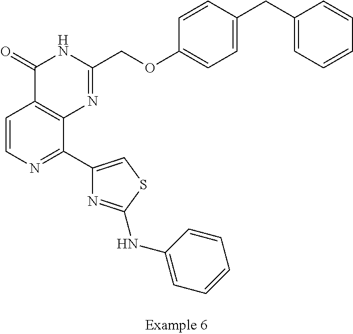 [SMILES: O=c1[nH]c(COc2ccc(Cc3ccccc3)cc2)nc2c(-c3csc(Nc4ccccc4)n3)nccc12]

Intermediate 22.

To a solution of Intermediate 20 (114 mg, 0.215 mmol) in 1,4-dioxane was added Tri-n-butyl(1-ethoxyvinyl)tin (156 mg, 0.431 mmol) and Tetrakis(triphenylphosphine)palladium(0) (25 mg, 0.0215 mmol). The mixture was heated to 140° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 22 (121 mg. qunt.).

Intermediate 23.

To a solution of Intermediate 22 (121 mg, 0.215 mmol) in tetrahydrofuran (5 ml) and water (1 ml) was added N-bromosuccinimide (34 mg, 0.194 mmol) at room temperature. The reaction mixture was allowed to stir for overnight and then diluted with water and extracted with ethyl acetate. The separated organic layer was dried over MgSO₄, filtered and concentrated in vauo. The concentrated residue was purified by flash column chromatography to afford the Intermediate 23 (123 mg, 0.200 mmol).

Intermediate 24.

To a solution of Intermediate 24 (106 mg, 0.172 mmol) in ethanol was added Phenylthiocarbamide (26 mg, 0.175 mmol). The reaction mixture was heated at reflux for overnight. After being cooled to room temperature, the mixture was concentrated in vacuo. The concentrated residue was purified by preparative HPLC to afford the Intermediate 24 (106 mg, 0.160 mmol).

### Compound of Example 6

The Intermediate 24 was dissolved in TFA (2 ml). The mixture was allowed to stir for 1 h at room temperature and then concentrated in vacuo. The concentrated residue was purified by preparative HPLC to afford the compound of Example 6.

### Example 7

### 8-(2-((2-hydroxyethyl)amino)thiazol-4-yl)-2-phenethylpyrido[3,4-d]pyrimidin-4(3H)-one

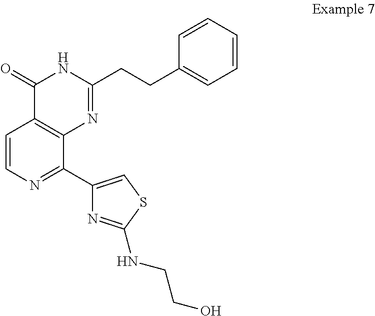 [SMILES: O=c1[nH]c(CCc2ccccc2)nc2c(-c3csc(NCCO)n3)nccc12]

Scheme for the preparation of the Compound of Example 7:

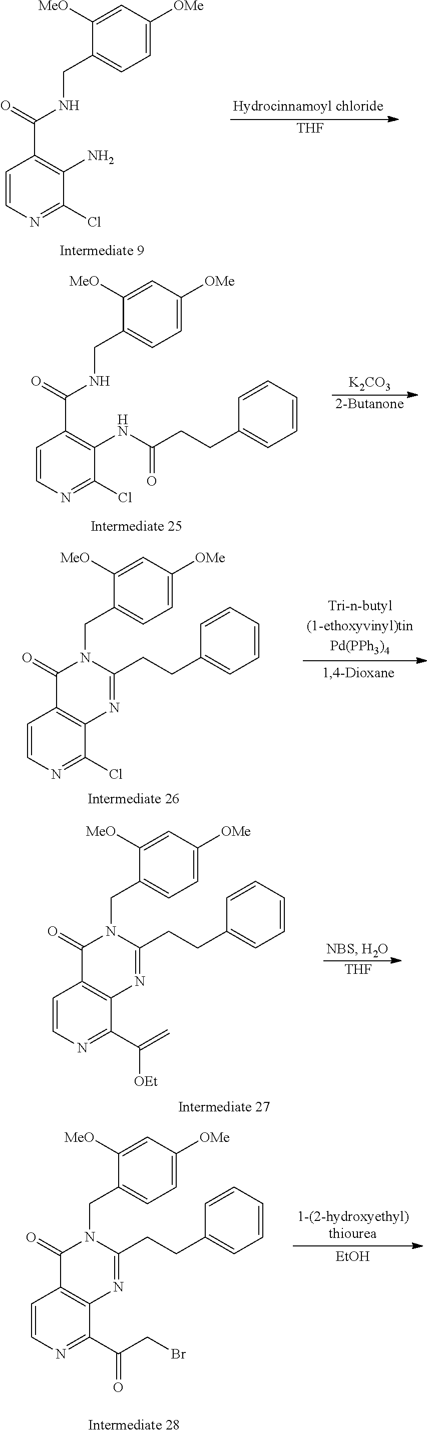

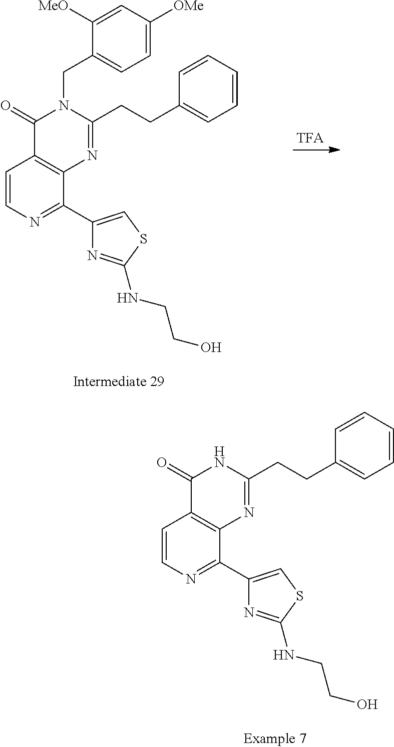 [SMILES: COc1ccc(Cn2c(CCc3ccccc3)nc3c(-c4csc(NCCO)n4)nccc3c2=O)c(OC)c1 ; O=c1[nH]c(CCc2ccccc2)nc2c(-c3csc(NCCO)n3)nccc12]

Intermediate 25.

To a solution of Intermediate 9 (450 mg, 1.398 mmol) in tetrahydrofuran was added hydrocinnamoyl chloride (208 ul, 1.398 mmol). The reaction mixture was allowed to stir for overnight. The precipitate was filtered off and washed with tetrahydrofuran. The solid was collected to afford Intermediate 25 (1.0 mmol) as pale yellow solid.

Intermediate 26.

To a suspension of Intermediate 25 (277 mg, 0.610 mmol) in 2-butanone was added potassium carbonate (253 mg, 1.830 mmol). The reaction mixture was heated at reflux and allowed to stir for overnight. After being cooled to ambient temperature, it was diluted with water and extracted with ethyl acetate. The organic extract was dried over magnesium sulfate and concentrated in vacuo. The concentrated residue was purified by combi-flash to afford Intermediate 26 (266 mg) as pale yellow oil.

Intermediate 27.

To a solution of Intermediate 26 (266 mg, 0.610 mmol) in 1,4-dioxane was added Tri-n-butyl(1-ethoxyvinyl)tin (412 ul, 1.22 mmol) and Tetrakis(triphenylphosphine)palladium(0) (96 mg, 0.0835 mmol). The mixture was heated to 120° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 27 (268 mg, 0.568 mmol).

Intermediate 28.

To a solution of Intermediate 27 (268 mg, 0.568 mmol) in tetrahydrofuran was added N-bromosuccinimide (101 mg, 0.568 mmol) at room temperature. The reaction mixture was allowed to stir for 30 minutes and then diluted with water and extracted with ethyl acetate. The separated organic layer was dried over MgSO₄, filtered and concentrated in vauo.

Intermediate 29.

To a solution of Intermediate 28 (0.2 mmol) in ethanol was added 1-(2-hydroxyethyl)thiourea (0.2 mmol). The reaction mixture was heated at reflux for 1 hour. After being cooled to room temperature, the mixture was concentrated in vacuo. The concentrated residue was purified by preparative HPLC to afford the Intermediate 29 (0.160 mmol).

### Compound of Example 7

The Intermediate 29 was dissolved in TFA (2 ml). The mixture was allowed to stir for 1 h at room temperature and then concentrated in vacuo. The concentrated residue was purified by preparative HPLC to afford the compound of Example 7.

### Example 8

### 8-(2-((2-hydroxyethyl)amino)thiazol-4-yl)-2-methylpyrido[3,4-d]pyrimidin-4(3H)-one

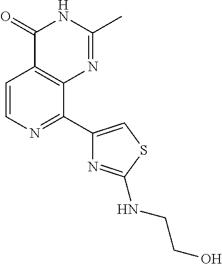 [SMILES: Cc1nc2c(-c3csc(NCCO)n3)nccc2c(=O)[nH]1]

Scheme for the preparation of the Compound of Example 8:

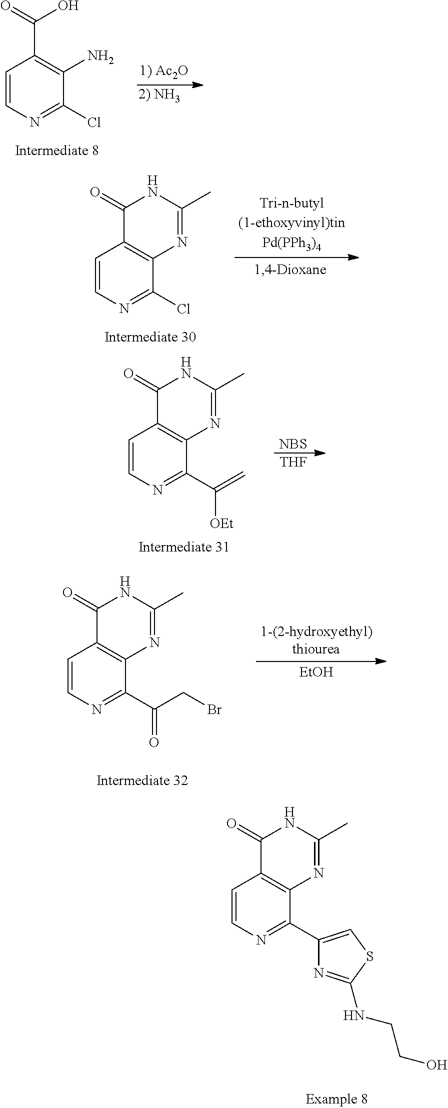

Intermediate 30.

A mixture of Intermediate 8 (156 mg, 0.903 mmol) and acetic anhydride (3 ml) was refluxed for 2 hours. After being cooled to room temperature, the mixture was concentrated in vacuo. To a crude product was added 15 N ammonia (5 ml), and then the mixture was stirred for 1 hours until in became clear. Additional stirring resulted in precipitates, which were filtered and dried to afford the Intermediate 30.

Intermediate 31.

To a solution of Intermediate 30 (0.4 mmol) in 1,4-dioxane was added Tri-n-butyl(1-ethoxyvinyl)tin (0.8 mmol) and Tetrakis(triphenylphosphine)palladium(0) (0.04 mmol). The mixture was heated to 120° C. for 30 minutes in a microwave. The mixture was then diluted with water and extracted with ethyl acetate. The separated organic layer was dried over MgSO₄, filtered and concentrated in vauo. The concentrated residue was purified by flash column chromatography to afford the Intermediate 31 (0.35 mmol).

Intermediate 32.

To a solution of Intermediate 31 (0.35 mmol) in tetrahydrofuran was added N-bromosuccinimide (0.35 mmol) at room temperature. The mixture action mixture was allowed to stir for 30 minutes and then diluted with water and extracted with ethyl acetate. The separated organic layer was dried over MgSO₄, filtered and concentrated in vauo.

### Compound of Example 8

To a solution of Intermediate 32 (0.3 mmol) in ethanol was added 1-(2-hydroxyethyl)thiourea (0.3 mmol). The reaction mixture was heated at reflux for 1 hour. After being cooled to room temperature, the mixture was concentrated in vacuo. The concentrated residue was purified by preparative HPLC to afford the compound of Example 8 (0.2 mmol).

### 3. Biochemical Testing

FLAG-tagged JARID1B (also known as KDM5B) protein was screened against 49 compounds. For this compound screening, LANCE Ultra time-resolved fluorescence resonance energy transfer (TR-FRET) assay was employed using a europoium (Eu)-labeled antibody that can specifically recognize mono- or dimethylated peptides (H3K4me2/1) and ULight-streptavidin (ULight-SA), a small molecule fluorescent dye. When irradiated at 340 nm, the energy from the Eu donor is transferred to the ULight acceptor dye which, in turn, emits light primarily at 665 nm. The ratio between the intensity of primary emission at 665 nm and that of secondary emission at 590 nm was used to quantify the level of lysine methylation. As JARID1B removes more methyl moieties from tri-methylated substrate peptides (H3K4me3), the ratio increases until the enzyme reaction is terminated. In case, therefore, the compounds disrupt the enzymatic activity completely, the ratio becomes equal to a background value.

Recombinant human JARID1B/KDM5B (accession number NP_006609) of molecular weight 179.9 KDa was expressed in Sf9 insect cells and contains an N-terminal FLAG tag. The enzyme was obtained from Active Motif (Catalog No. 31432). The tri-methylated histone substrate peptide of purity greater than 95% was a synthetic peptide in which the first 21 amino acids correspond to the human histone H3 sequence with three extra amino acids and a biotin motif (GGK-biotin) linked to the C-terminus [sequence: ART-K(Me3)-QTARKSTGGKAPRKQLA-GGK-biotin-OH], (AnaSpec, Fremont, Calif. Catalog. No. ANA-1413). The reference inhibitor compound tranylcypromine (or trans-2-phenylcyclopropylamine hydrochloride, also known as, 2-PCPA) was purchased from Sigma (St. Louis, Mo. Cat. no. P8511). ULight-labeled streptavidin (ULight-SA, Catalog no. TRF0102), Eu-W1024-labeled anti-methyl-Histone H3 Lysine 4 (H3K4me1-2) Antibody (Catalog no. TRF0402), and LANCE detection buffer 10× (Catalog no. CR97-100) were obtained from PerkinElmer (Montreal, Quebec, Canada). The TR-FRET experiments were carried out in white, low-volume 384-wellplates purchased from PerkinElmer (ProxiPlate_-384 Plus, Catalog no. 6008280).

The TR-FRET signal was measured in the presence both of the bio-H3K4me3 peptide and FLAG-JARID1B by detecting any H3K4me2/1 peptide produced in the assay system. Assays using only the bio-H3K4me2 peptide were served as a positive control. Robust enzymatic progressions were observed by using JARID1B at concentrations ranging from 10 to 30 nM. In a typical TR-FRET experiment, JARID1B was pre-incubated with or without 0.1 uM of test-compounds (containing 1% DMSO final) for 5 min. The enzymatic reactions were initiated by the addition of 500 nM biotinylated H3K4me3 peptide substrate plus 500 uM of 2-OG 25 uM Fe(II) and 2 mM ascorbate. The reaction buffer also contained 50 mM Hepes (pH7.5), 0.01% (v/v) Tween 20, and 50 mM NaCl. The reaction mixture was incubated for 30 min at room temperature before reading on an EnVision plate reader (PerkinElmer, Waltham, Mass.). Results are seen Table 2.

TABLE 2

| No. | % Inhibition |
|---|---|
| 1 | + |
| 2 | + |
| 3 | + |
| 4 | + |
| 5 | + |
| 6 | + |
| 7 | + |
| 8 | + |
| 9 | + |
| 10 | + |
| 11 | + |
| 12 | + |
| 13 | + |
| 14 | ++ |
| 15 | ++ |
| 16 | + |
| 17 | ++ |
| 18 | ++ |
| 19 | ++ |
| 20 | +++ |
| 21 | +++ |
| 22 | +++ |
| 23 | +++ |
| 24 | +++ |
| 25 | ++ |
| 26 | ++ |
| 27 | ++ |
| 28 | +++ |
| 29 | ++ |
| 30 | ++ |
| 31 | +++ |
| 32 | ++ |
| 33 | ++ |
| 34 | ++ |
| 35 | ++ |
| 36 | ++ |
| 37 | + |
| 38 | +++ |
| 39 | +++ |
| 40 | +++ |
| 41 | +++ |
| 42 | +++ |
| 43 | ++ |
| 44 | ++ |
| 45 | ++ |
| 46 | ++ |
| 47 | ++ |
| 48 | ++ |
| 49 | ++ |
| 50 | ++ |
| 51 | +++ |
| 52 | +++ |
| 53 | +++ |
| 54 | +++ |
| 55 | +++ |
| 56 | +++ |
| 57 | ++ |
| 58 | ++ |
| 59 | ++ |
| 60 | ++ |
| 61 | ++ |
| 62 | + |
| 63 | ++ |
| 64 | ++ |
| 65 | ++ |
| 66 | + |
| 67 | ++ |
| 68 | + |
| 69 | + |
| 70 | ++ |
| 71 | +++ |
| 72 | ++ |
| 73 | + |
| 74 | ++ |
| 75 | + |
| 76 | + |
| 77 | ++ |
| 78 | + |
| 79 | ++ |
| 80 | ++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | ++ |
| 96 | + |
| 97 | ++ |
| 98 | ++ |
| 99 | ++ |
| 100 | ++ |
| 101 | ++ |
| 102 | +++ |
| 103 | + |
| 104 | ++ |
| 105 | + |
| 106 | ++ |
| 107 | ++ |
| 108 | + |
| 109 | + |
| 110 | + |
| 111 | ++ |
| 112 | + |
| 113 | +++ |
| 114 | + |
| 115 | + |
| 116 | + |
| 117 | + |
| 118 | ++ |
| 119 | ++ |
| 120 | ++ |
| 121 | ++ |
| 122 | + |
| 123 | + |
| 124 | + |
| 125 | +++ |
| 126 | +++ |
| 127 | +++ |
| 128 | + |
| 129 | + |
| 130 | +++ |
| 131 | +++ |
| 132 | ++ |
| 133 | ++ |
| 134 | ++ |
| 135 | ++ |
| 136 | +++ |
| 137 | + |
| 138 | + |
| 139 | + |
| 140 | ++ |
| 141 | + |
| 142 | ++ |
| 143 | ++ |
| 144 | + |
| 145 | + |
| 146 | + |
| 147 | + |
| 148 | + |
| 149 | ++ |
| 150 | + |
| 151 | + |
| 152 | + |
| 153 | + |
| 154 | + |
| 155 | ++ |
| 156 | ++ |
| 157 | + |
| 158 | + |
| 159 | + |
| 160 | ++ |
| 161 | ++ |
| 162 | ++ |
| 163 | ++ |
| 164 | +++ |
| 165 | ++ |
| 166 | ++ |
| 167 | ++ |
| 168 | + |

NOTE:
+++: % Inhibition ≧ 50,
++: 50 > % Inhibition ≧ 20,
+: 20 > % Inhibition

As used herein, the term “2,4-PDCA” refers to 2,4-pyridinedicarboxylic acid monohydrate.

As used herein, the term “DMSO” refers to dimethyl sulfoxide.

As used herein, the term “bio” refers to biotin or biotinylated

As used herein, the term “H3K4me2” refers to dimethylated lysine 4 in histone H3

As used herein, the term “H3K4me3” refers to trimethylated lysine 4 in histone H3.

As used herein, the term “KDM5” refers to Lysine Demethylase 5

As used herein, the term “a-KG” refers to alpha-ketoglutarate, or a salt or solvate thereof.

As used herein, the term “IC₅₀” refers to half maximal inhibitory concentration

### 4. Cellular Testing

In order to test the cellular inhibitory potency of compounds, immunoblot analyses were performed to assess the level of global trimethylation at lysine 4 on histone H3 in a human osteosarcoma U2-OS cell line stably overexpressing KDM5B.

U2-OS cells were seeded in 6-well plates at a density of 2.5×10⁵ cells/well in 3 mL McCoy's 5A medium containing 10% heat-inactivated fetal bovine serum and 100 U/ml penicillin/streptomycin (Invitrogen Gibco, USA) and incubated overnight. A KDM5B-expression plasmid tagged with Myc-DDK (Origene, USA) was transfected into the cells using Lipofectamine 2000 (Invitrogen, USA) according to the manufacturer's instructions. Forty-eight hours after the transfection, the cells were diluted to 1:100 for passage and neomycin-resistant clones were selected in the presence of 600 μg/ml G418 (Gibco BRL, USA) for 2 weeks. The positive clones were picked up and expanded individually for 2 weeks. The expression of KDM5B in each clone was verified by immunoblot analysis, and the clones overexpressing KDM5B were subsequently maintained in McCoy's 5A medium supplemented with 300 μg/ml G418 at 37° C. in an atmosphere of 5% CO₂.

For assays of compounds, U2-OS cells stably overexpressing KDM5B were seeded in 12-well plates at a density of 1.0×10⁵ cells/well in 1 mL of McCoy's 5A medium without G418. The cells were incubated for 24 hours before the addition of compounds. The compounds were diluted in McCoy's 5A medium and the total volume of medium in each well was 2 mL with the final concentration of DMSO 0.3%.

Twenty-four hours after the treatment with compounds, the cells were washed twice with Dulbecco's Phosphate-Buffered Saline and total cellular proteins were extracted with RIPA buffer (Simga, USA) containing protease inhibitor (Complete Protease Inhibitor Cocktail Tablets; Roche Applied Science, Switzerland). The extract was centrifuged at 14,000×g for 10 minutes, and the supernatants were recovered. The protein concentration was quantitated using BCA Protein Assay (Pierce, USA) and SoftMax pro software version 5.2 (Molecular Device, USA). After denaturation at 95° C. for 10 minutes, the total proteins (20 μg of protein/lane) were separated by SDS-PAGE on 4˜12% gradient gels (Invitrogen, USA). The resolved proteins were transferred onto a 0.45-μm nitrocellulose membrane by wet electroblotting for 1 hour, and then the membrane was soaked in Tris-buffered saline containing 5% nonfat dry milk and 0.05% Tween-20 (TBS-T) for 1 hour at room temperature. The membrane was incubated with 1:2000 H3K4me3 antibodies (ab8580; Abcam) and 1:10000 H3 antibodies (ab1791; Abcam) overnight at 4° C. The membrane was washed with TBS-T three times for 30 min and then incubated with 1:5000 or 1:20000 anti-rabbit secondary antibodies for 1 hour at room temperature. Protein bands of interest were visualized by chemiluminescence (Amersham ECL prime Western Blotting Detection Reagents; GE Healthcare Lifesciences, USA). Each protein band image was acquired by a Molecular Imager ChemiDoc XRS System (Bio-Rad, USA) and quantified by using Quantity One software (ver4.6.7). The normalized level of tri-methylation was determined by dividing an H3K4me3 band intensity by a corresponding H3 band intensity and half-maximal inhibitory concentration (IC50) was calculated using Sigma Plot.

What is claimed is:

1. A compound represented by following Formula 1, an isomer thereof, or a pharmaceutically acceptable salt thereof: 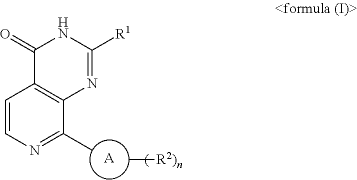
wherein:
R¹ is selected from H, halogen, alkyl, alkoxy, alkenyl, carbocyclyl, aryl, heterocyclyl, heteroaryl, —C(O)R^{b}, —C(O)OR^{b} and —C(O)N(R^{b})₂, —CH₂—O-aryl, —CH₂—O-biaryl, wherein each alkyl, alkenyl, carbocyclyl, aryl, heterocyclyl, heteroaryl, —CH₂—O-aryl or —CH₂—O-biaryl of R¹ is optionally substituted with one or more R^{x};
A is heteroaryl that is substituted with one or more R², wherein heteroaryl is monocyclic or bicyclic ring; and n is from 1 to 6;
R² is independently selected from H, halogen, alkyl, alkoxy, alkenyl, carbocyclyl, aryl, heterocyclyl, heteroaryl, —OR^{b}, —SR^{b}, —N(R^{b})₂, —NR^{b}C(O)R^{b}, —NHC(O)OR^{b}, —NHC(O)NHR^{b}, —C(O)R^{b}, —C(O)OR^{b}, —C(O)N(R^{b})₂, wherein each alkyl, alkenyl, carbocyclyl, aryl, heterocyclyl or heteroaryl of R² is optionally substituted with one or more R^{x};
R^{b} is selected from H, alkyl, alkenyl, carbocyclyl, aryl, heterocyclyl and heteroaryl, wherein each alkyl, alkenyl, carbocyclyl, and heterocyclyl is optionally substituted with one or more R^{x};
each R^{x} is selected from halogen, alkyl, alkenyl, carbocyclyl, aryl, heterocyclyl, heteroaryl, —NO₂, —N(R^{y})₂, —CH₂—N(R^{y})₂, —CN, —C(O)—N(R^{y})₂, —S(O)—N(R^{y})₂, —S(O)₂—N(R^{y})₂, —O-aryl, —O-heteroaryl, —O—R^{y}, —S—R^{y}, —O—C(O)—R^{y}, —O—C(O)—O—R^{y}, —C(O)—R^{y}, —C(O)—O—R^{y}, —S(O)—R^{y}, —S(O)₂—R^{y}, —O—C(O)—N(R^{y})₂, —N(R^{y})—C(O)—OR^{y}, —N(R^{y})—S(O)—N(R^{y})₂, and —N(R^{y})—S(O)₂—N(R^{y})₂, wherein alkyl, alkenyl, carbocyclyl, aryl, heterocyclyl or heteroaryl of R^{x} is optionally substituted with one or more groups independently selected from the group consisting of halogen, alkyl, alkoxy, hydroxy, and aryl;
each R^{y} is selected from H, alkyl, alkenyl, carbocyclyl, aryl, heterocyclyl, heteroaryl, —C(O)R^{z}, —C(O)OR^{z}, —C(O)N(R^{z})₂, —CH₂—R^{z}, that is optionally substituted with one or more groups independently selected from the group consisting of halogen, alkyl, carbocyclyl and heterocyclyl;
each R^{z} is selected from carbocyclyl, aryl, heterocyclyl, heteroaryl, alkylcarbocyclyl, alkylheterocyclyl, wherein each carbocyclyl, aryl, heterocyclyl, heteroaryl, alkylcarbocyclyl, alkylheterocyclyl is optionally substituted with one or more groups independently selected from the group consisting of alkyl, alkoxy, hydroxy, hydroxyalkyl, amino.

2. The compound of claim 1, the isomer thereof, or the pharmaceutically acceptable salt thereof, wherein
R¹ is selected from H, halogen, C₁₋₆alkyl, C₁₋₆alkoxy, C₂₋₆alkenyl, C₃₋₈carbocyclyl, C₆₋₁₀ aryl, 4- to 8-membered heterocyclyl including 1 to 3 hetero atoms selected from the group consisting of N, O and S, 4- to 8-membered heteroaryl including 1 to 3 hetero atoms selected from the group consisting of N, O and S, —C(O)R^{b}, —C(O)OR^{b} and —C(O)N(R^{b})₂, —CH₂—O—C₆₋₁₀ aryl, —CH₂—O-bi-C₆₋₁₀ aryl, wherein each C₁₋₆alkyl, C₂₋₆alkenyl, C₃₋₈carbocyclyl, C₅₋₁₀aryl, 4- to 8-membered heterocyclyl including 1 to 3 hetero atoms selected from the group consisting of N, O and S, 4- to 8-membered heteroaryl including 1 to 3 hetero atoms selected from the group consisting of N, O and S, —CH₂—O—C₅₋₁₀ aryl or —CH₂—O-bi-C₅₋₁₀ aryl of R¹ is optionally substituted with one or more R^{x};
A is 4- to 8-membered heteroaryl including 1 to 3 hetero atoms selected from the group consisting of N, O and S, that is substituted with one or more R², wherein heteroaryl is monocyclic or bicyclic ring; and n is from 1 to 4;
R² is independently selected from H, F, Cl, Br, C₁₋₆alkyl, C₁₋₆alkoxy, C₂₋₆alkenyl, C₃₋₈carbocyclyl, C₆₋₁₀ aryl, 4- to 8-membered heterocyclyl including 1 to 3 hetero atoms selected from the group consisting of N, O and S, 4- to 8-membered heteroaryl including 1 to 3 hetero atoms selected from the group consisting of N, O and S, —OR^{b}, —SR^{b}, —N(R^{b})₂, —NR^{b}C(O)R^{b}, —NHC(O)OR^{b}, —NHC(O)NHR^{b}, —C(O)R^{b}, —C(O)OR^{b}, —C(O)N(R^{b})₂, wherein each C₁₋₆ alkyl, C₁₋₆alkoxy, C₂₋₆alkenyl, carbocyclyl, C₆₋₁₀ aryl, 4- to 8-membered heterocyclyl including 1 to 3 hetero atoms selected from the group consisting of N, O and S or 4- to 8-membered heteroaryl including 1 to 3 hetero atoms selected from the group consisting of N, O and S of R² is optionally substituted with one or more R^{x};
R^{b} is selected from the group consisting of H, C₁₋₆alkyl, C₂₋₆alkenyl, C₃₋₈carbocyclyl, C₆₋₁₀ aryl, 4- to 8-membered heterocyclyl including 1 to 3 hetero atoms selected from the group consisting of N, O and S and 4- to 8-membered heteroaryl including 1 to 3 hetero atoms selected from the group consisting of N, O and S, wherein each C₁₋₆alkyl, C₂₋₆alkenyl, C₃₋₈carbocyclyl, C₆₋₁₀ aryl, 4- to 8-membered heterocyclyl including 1 to 3 hetero atoms selected from the group consisting of N, O and S and 4- to 8-membered heteroaryl including 1 to 3 hetero atoms selected from the group consisting of N, O and S is optionally substituted with one or more R^{x};
R^{x} is selected from halogen, C₁₋₆alkyl, C₂₋₆alkenyl, C₃₋₈carbocyclyl, C₆₋₁₀ aryl, heteroaryl, heterocyclyl, —NO₂, —N(R^{y})₂, —CH₂—N(R^{y})₂, —CN, —C(O)—N(R^{y})₂, —S(O)—N(R^{y})₂, —S(O)₂—N(R^{y})₂, —O—C₆₋₁₀ aryl, —O-heteroaryl, —O—R^{y}, —S—R^{y}, —O—C(O)—R^{y}, —O—C(O)—O—R^{y}, —C(O)—R^{y}, —C(O)—O—R^{y}, —S(O)—R^{y}, —S(O)₂—R^{y}, —O—C(O)—N(R^{y})₂, —N(R^{y})—C(O)—OR^{y}, —N(R^{y})—S(O)—N(R^{y})₂, and —N(R^{y})—S(O)₂—N(R^{y})₂, wherein alkyl, alkenyl, carbocyclyl, aryl, heterocyclyl or heteroaryl of R^{x} is optionally substituted with one or more groups independently selected from halogen, alkyl, alkoxy and hydroxy; and heteroaryl, and heterocyclyl are independently 4- to 8-membered ring including 1 to 3 hetero atoms selected from the group consisting of N, O and S;
R^{y} is selected from the group consisting of H, C₁₋₆alkyl, C₂₋₆alkenyl, C₃₋₈carbocyclyl, C₆₋₁₀ aryl, 4- to 8-membered heterocyclyl including 1 to 3 hetero atoms selected from the group consisting of N, O and S and 4- to 8-membered heteroaryl including 1 to 3 hetero atoms selected from the group consisting of N, O and S, —C(O)R^{z}, —C(O)OR^{z}, —C(O)N(R^{z})₂, —CH₂—R^{z}, that is optionally substituted with one or more groups independently selected from F, Cl, Br, C₁₋₄alkyl, C₃₋₈carbocyclyl and 4- to 8-membered heterocyclyl including 1 to 3 hetero atoms selected from the group consisting of N, O and S; and
R^{z} is selected from the group consisting of C₃₋₈ carbocyclyl, C₆₋₁₀ aryl, heterocyclyl, heteroaryl, C₁₋₄ alkyl C₃₋₈ carbocyclyl, C₁₋₄ alkylheterocyclyl, wherein each C₃₋₈ carbocyclyl, C₆₋₁₀ aryl, heterocyclyl, heteroaryl, C₁₋₄ alkyl C₃₋₈ carbocyclyl and C₁₋₄ alkylheterocyclyl is optionally substituted with one or more groups independently selected from C₁₋₄ alkyl, C₁₋₄ alkoxy, hydroxy, hydroxy C₁₋₄ alkyl and amino, and
heteroaryl, and heterocyclyl are independently 4- to 8-membered ring including 1 to 3 hetero atoms selected from the group consisting of N, O and S.

3. The compound of claim 1, the isomer thereof, or the pharmaceutically acceptable salt thereof, wherein
R¹ is selected from H, F, Cl, methyl, ethyl, propyl, isopropyl, butyl, isobutyl, n-butyl, ethenyl, benzyl, CH₂O-phenyl, CH₂O-biphenyl and C(O)OCH₃, wherein each methyl, ethyl, propyl, isopropyl, butyl, isobutyl, n-butyl, ethenyl, benzyl, CH₂O-phenyl and CH₂O-biphenyl is optionally substituted with one or two groups selected from H, F, Cl, cyclopentyl, phenyl, benzyl, 2-phenylpropyl and benzoyl;
A is pyrazole, imidazole, thiazole, oxazole, thiadiazole, oxadiazole, triazole, dithiazole, dioxazole, pyrimidine, pyrazine or pyridazine that is substituted with one or more R²; and n is from 1 to 2;
R² is independently selected from H, F, Cl, Br, C₁₋₃alkyl, C₁₋₃alkoxy, C₂₋₄alkenyl, cyclopentyl, cyclohexyl, phenyl, pyridine, pyrazole, imidazole, oxazole, pyrimidine, piperidine, piperazine, pyrrolidine, morpholine, —OR^{b}, —SR^{b}, —N(R^{b})₂, —NR^{b}C(O)R^{b}, —NHC(O)OR^{b}, —NHC(O)NHR^{b}, —C(O)R^{b}, —C(O)OR^{b}, —C(O)N(R^{b})₂, wherein each C₁₋₃alkyl, C₁₋₃alkoxy, C₂₋₄alkenyl, phenyl, pyrazole, pyrimidine, piperidine and morpholine is optionally substituted with one or more R^{x};
R^{b} is selected from the group consisting of H, methyl, ethyl, propyl, butyl, pentyl, propenyl, phenyl, pyrrole, pipeazine, piperidine and cyclohexyl, wherein each H, methyl, ethyl, propyl, butyl, pentyl, propenyl, phenyl, pyrrole, pipeazine, piperidine and cyclohexyl is optionally substituted with one or more R^{x};
R^{x} is selected from F, Cl, Br, OH, methyl, ethyl, propyl, cyclopentyl, cyclohexyl, phenyl, —C(O)—R^{y}, —N(R^{y})₂, wherein methyl, ethyl, propyl, cyclopentyl, cyclohexyl or phenyl, of R^{x} is optionally substituted with one or more groups independently selected from F, Cl, Br, C₁₋₄alkyl, C₁₋₄alkoxy, and hydroxy;
R^{y} is selected from the group consisting of H, methyl, ethyl, isopropyl, cyclopentyl, phenyl, —C(O)R^{z}, —C(O)OR^{z}, —C(O)N(R^{z})₂, and —CH₂—R^{z}, that is optionally substituted with one or more groups independently selected from F, Cl, Br, hydroxyl, methoxy, methyl and ethyl;
R^{z} is selected from the group consisting of phenyl and piperazine, which is optionally substituted with one or more groups independently selected from C₁₋₄ alkyl, C₁₋₄ alkoxy, hydroxy, hydroxy C₁₋₄ alkyl and amino.

4. The compound of claim 1, the isomer thereof, or the pharmaceutically acceptable salt thereof, wherein
R² is independently selected from the group consisting of H, methyl, chloro, tolyl, methylpiperazinylmethylphenyl, hydroxymethylphenyl, fluorophenyl, chlorophenyl, benzyloxyphenyl, hydroxypropylphenyl, methoxybenzyloxyphenyl, phenoxyphenyl, methylpyrazolyl, pyridinyl, benzylpyrazolyl, piperazinylpyridinyl, piperazinylphenyl, morpholinocarbonylphenyl, piperidinlypyrazolyl, acetylphenyl, hydroxyphenyl, hydroxyethylpyrazolyl, pyrrolidinylethylpyrazolyl, morpholinoethylpyrazolyl, methylpiperazinylethylpyrazolyl, methylpiperazinylphenyl, methylpiperazinylbenzyl, pyrrolidinylmethylphenyl, cylopentylpyrazolyl, methylcarbonylaminophenyl, amino, hydroxyethylamino, hydroxypropylamino, dihydroxypropylamino, dihydroxypropanylamino, hydroxybutylamino, tetrahydrofuranylamino, tetrahydropyranylamino, methoxyethylamino, dimethylaminoethylamino, dimethylaminopropylamino, aminoethylamino, methylpiperazinylethylamino, methylpyrrolidinylmethylamino, methylpyrrolidinylethylamino, piperidinylamino, methylpiperidinylamino, isopropylpiperidinylmethylamino, aminocyclohexylamino, hydroxybutylamino, methylmorpholinylmethylamino, hydroxyethylpiperidinylmethylamino, benzylpiperidinylamino, ethoxycarbonyl, hydroxyethyl, benzyl, morpholinoethyl, benzylpiperidinylmethylamino, methoxyethylpiperidinylmethylamino, cyclopentylpiperidinylmethylamino, allylamino, phenylamino, morpholinyl, morpholinoethylamino, diethylamino, benzylpyrrolidinylamino, dichlorophenethylamino, chlorophenylpropylamino, phenyoxyphenethylamino, hydroxy(pyridinyl)ethylamino, dimethylphenethylethylamino, hydroxy(piperazinyl)propylamino, cyclohexylmethylamino, diethylaminopentylamino, pyrrolidinylbutylamino, diisopropylpentylamino, hydroxypentanylamino, ethoxycarbonyl, phenoxypropylamino and benzoylamino.

5. The compound of claim 1, the isomer thereof, or the pharmaceutically acceptable salt thereof, wherein it is selected from the group consisting of the following compounds:
1) 8-(4-(m-tolyl)-1H-pyrazol-1-yl)pyrido[3,4-d]pyrimidin-4(3H)-one;
2) 8-(4-(4-((4-methylpiperazin-1-yl)methyl)phenyl)-1H-pyrazol-1-yl)pyrido[3,4-d]pyrimidin-4(3H)-one;
3) 8-(4-(3-(hydroxymethyl)phenyl)-1H-pyrazol-1-yl)pyrido[3,4-d]pyrimidin-4(3H)-one;
4) 8-(4-(4-fluorophenyl)-1H-pyrazol-1-yl)pyrido[3,4-d]pyrimidin-4(3H)-one;
5) 8-(4-(3-(benzyloxy)phenyl)-1H-pyrazol-1-yl)pyrido[3,4-d]pyrimidin-4(3H)-one;
6) 8-(4-(3-((4-methylpiperazin-1-yl)methyl)phenyl)-1H-pyrazol-1-yl)pyrido[3,4-d]pyrimidin-4(3H)-one;
7) 8-(4-(3-(3-hydroxypropyl)phenyl)-1H-pyrazol-1-yl)pyrido[3,4-d]pyrimidin-4(3H)-one;
8) 8-(4-(3-((3-methoxybenzyl)oxy)phenyl)-1H-pyrazol-1-yl)pyrido[3,4-d]pyrimidin-4(3H)-one;
9) 8-(4-(2-(hydroxymethyl)phenyl)-1H-pyrazol-1-yl)pyrido[3,4-d]pyrimidin-4(3H)-one;
10) 8-(4-(2-phenoxyphenyl)-1H-pyrazol-1-yl)pyrido[3,4-d]pyrimidin-4(3H)-one;
11) 8-(1′-methyl-1H,1′H-[4,4′-bipyrazol]-1-yl)pyrido[3,4-d]pyrimidin-4(3H)-one;
12) 8-(4-(pyridin-3-yl)-1H-pyrazol-1-yl)pyrido[3,4-d]pyrimidin-4(3H)-one;
13) 8-(1′-benzyl-1H,1′H-[4,4′-bipyrazol]-1-yl)pyrido[3,4-d]pyrimidin-4(3H)-one;
14) 8-(3-amino-4-(6-(piperazin-1-yl)pyridin-3-yl)-1H-pyrazol-1-yl)pyrido[3,4-d]pyrimidin-4(3H)-one;
15) 8-(3-amino-1′-methyl-1H,1′H-[4,4′-bipyrazol]-1-yl)pyrido[3,4-d]pyrimidin-4(3H)-one;
16) 8-(3-amino-4-(pyridin-3-yl)-1H-pyrazol-1-yl)pyrido[3,4-d]pyrimidin-4(3H)-one;
17) 8-(3-amino-4-(4-(4-methylpiperazin-1-yl)phenyl)-1H-pyrazol-1-yl)pyrido[3,4-d]pyrimidin-4(3H)-one;
18) N-(4-(3-amino-1-(4-oxo-3,4-dihydropyrido[3,4-d]pyrimidin-8-yl)-1H-pyrazol-4-yl)phenyl)acetamide;
19) 8-(3-amino-4-(4-(3-hydroxypropyl)phenyl)-1H-pyrazol-1-yl)pyrido[3,4-d]pyrimidin-4(3H)-one;
20) 8-(3-amino-4-(3-(piperazin-1-yl)phenyl)-1H-pyrazol-1-yl)pyrido[3,4-d]pyrimidin-4(3H)-one;
21) 8-(3-amino-4-(4-(morpholine-4-carbonyl)phenyl)-1H-pyrazol-1-yl)pyrido[3,4-d]pyrimidin-4(3H)-one;
22) 8-(3-amino-1′-(piperidin-4-yl)-1H,1′H-[4,4′-bipyrazol]-1-yl)pyrido[3,4-d]pyrimidin-4(3H)-one;
23) 8-(3-amino-4-(4-fluorophenyl)-1H-pyrazol-1-yl)pyrido[3,4-d]pyrimidin-4(3H)-one;
24) 8-(4-(4-acetylphenyl)-3-amino-1H-pyrazol-1-yl)pyrido[3,4-d]pyrimidin-4(3H)-one;
25) 8-(3-amino-4-(4-hydroxyphenyl)-1H-pyrazol-1-yl)pyrido[3,4-d]pyrimidin-4(3H)-one;
26) 8-(3-amino-1′-(2-hydroxyethyl)-1H,1′H-[4,4′-bipyrazol]-1-yl)pyrido[3,4-d]pyrimidin-4(3H)-one;
27) 8-(3-amino-1′-(2-(pyrrolidin-1-yl)ethyl)-1H, 1′H-[4,4′-bipyrazol]-1-yl)pyrido[3,4-d]pyrimidin-4(3H)-one;
28) 8-(3-amino-1′-benzyl-1H,1′H-[4,4′-bipyrazol]-1-yl)pyrido[3,4-d]pyrimidin-4(3H)-one;
29) 8-(3-amino-1′-(2-morpholinoethyl)-1H,1′H-[4,4′-bipyrazol]-1-yl)pyrido[3,4-d]pyrimidin-4(3H)-one;
30) 8-(3-amino-1′-(2-(4-methylpiperazin-1-yl)ethyl)-1H,1′H-[4,4′-bipyrazol]-1-yl)pyrido[3,4-d]pyrimidin-4(3H)-one;
31) 8-(3-amino-4-(3-(benzyloxy)phenyl)-1H-pyrazol-1-yl)pyrido[3,4-d]pyrimidin-4(3H)-one;
32) 8-(3-amino-4-(3-chlorophenyl)-1H-pyrazol-1-yl)pyrido[3,4-d]pyrimidin-4(3H)-one;
33) 8-(3-amino-4-(4-((4-methylpiperazin-1-yl)methyl)phenyl)-1H-pyrazol-1-yl)pyrido[3,4-d]pyrimidin-4(3H)-one;
34) 8-(3-amino-4-(4-(pyrrolidin-1-ylmethyl)phenyl)-1H-pyrazol-1-yl)pyrido[3,4-d]pyrimidin-4(3H)-one;
35) 8-(3-amino-1′-cyclopentyl-1H,1′H-[4,4′-bipyrazol]-1-yl)pyrido[3,4-d]pyrimidin-4(3H)-one;
36) 8-(3-amino-4-(3-(hydroxymethyl)phenyl)-1H-pyrazol-1-yl)pyrido[3,4-d]pyrimidin-4(3H)-one;
37) 8-(3-amino-4-(3-((4-methylpiperazin-1-yl)methyl)phenyl)-1H-pyrazol-1-yl)pyrido[3,4-d]pyrimidin-4(3H)-one;
38) 8-(2-((2-hydroxyethyl)amino)thiazol-4-yl)pyrido[3,4-d]pyrimidin-4(3H)-one;
39) (S)-8-(2-((2-hydroxypropyl)amino)thiazol-4-yl)pyrido[3,4-d]pyrimidin-4(3H)-one;
40) (R)-8-(2-((2-hydroxypropyl)amino)thiazol-4-yl)pyrido[3,4-d]pyrimidin-4(3H)-one;
41) 8-(2-(((tetrahydrofuran-2-yl)methyl)amino)thiazol-4-yl)pyrido[3,4-d]pyrimidin-4(3H)-one;
42) 8-(2-((2-methoxyethyl)amino)thiazol-4-yl)pyrido[3,4-d]pyrimidin-4(3H)-one;
43) 8-(2-((2-(dimethylamino)ethyl)amino)thiazol-4-yl)pyrido[3,4-d]pyrimidin-4(3H)-one;
44) 8-(2-((2-aminoethyl)amino)thiazol-4-yl)pyrido[3,4-d]pyrimidin-4(3H)-one;
45) 8-(2-((2-(4-methylpiperazin-1-yl)ethyl)amino)thiazol-4-yl)pyrido[3,4-d]pyrimidin-4(3H)-one;
46) 8-(2-((3-hydroxypropyl)amino)thiazol-4-yl)pyrido[3,4-d]pyrimidin-4(3H)-one;
47) 8-(2-((3-(dimethylamino)propyl)amino)thiazol-4-yl)pyrido[3,4-d]pyrimidin-4(3H)-one;
48) 8-(2-((2-(1-methylpyrrolidin-2-yl)ethyl)amino)thiazol-4-yl)pyrido[3,4-d]pyrimidin-4(3H)-one;
49) 8-(2-(piperidin-4-ylamino)thiazol-4-yl)pyrido[3,4-d]pyrimidin-4(3H)-one;
50) 8-(2-((1-methylpiperidin-4-yl)amino)thiazol-4-yl)pyrido[3,4-d]pyrimidin-4(3H)-one;
51) 8-(2-(((1-(2-hydroxyethyl)piperidin-4-yl)methyl)amino)thiazol-4-yl)pyrido[3,4-d]pyrimidin-4(3H)-one;
52) 8-(2-(((1-benzylpiperidin-4-yl)methyl)amino)thiazol-4-yl)pyrido[3,4-d]pyrimidin-4(3H)-one;
53) 8-(2-(((1-(2-methoxyethyl)piperidin-4-yl)methyl)amino)thiazol-4-yl)pyrido[3,4-d]pyrimidin-4(3H)-one;
54) 8-(2-(((1-cyclopentylpiperidin-4-yl)methyl)amino)thiazol-4-yl)pyrido[3,4-d]pyrimidin-4(3H)-one;
55) 8-(2-(((1-isopropylpiperidin-4-yl)methyl)amino)thiazol-4-yl)pyrido[3,4-d]pyrimidin-4(3H)-one;
56) 8-(2-((piperidin-4-ylmethyl)amino)thiazol-4-yl)pyrido[3,4-d]pyrimidin-4(3H)-one;
57) 8-(2-(((1s,4s)-4-aminocyclohexyl)amino)thiazol-4-yl)pyrido[3,4-d]pyrimidin-4(3H)-one;
58) 8-(2-(((4-methylmorpholin-2-yl)methyl)amino)thiazol-4-yl)pyrido[3,4-d]pyrimidin-4(3H)-one;
59) 8-(2-(((1-methylpyrrolidin-3-yl)methyl)amino)thiazol-4-yl)pyrido[3,4-d]pyrimidin-4(3H)-one;
60) 8-(2-((2-hydroxybutyl)amino)thiazol-4-yl)pyrido[3,4-d]pyrimidin-4(3H)-one;
61) 8-(2-((1-benzylpiperidin-4-yl)amino)thiazol-4-yl)pyrido[3,4-d]pyrimidin-4(3H)-one;
62) ethyl 4-(4-oxo-3,4-dihydropyrido[3,4-d]pyrimidin-8-yl)thiazole-2-carboxylate;
63) 8-(1-(2-hydroxyethyl)-1H-pyrazol-4-yl)pyrido[3,4-d]pyrimidin-4(3H)-one;
64) 8-(1-benzyl-1H-pyrazol-4-yl)pyrido[3,4-d]pyrimidin-4(3H)-one;
65) 8-(1-(2-morpholinoethyl)-1H-pyrazol-4-yl)pyrido[3,4-d]pyrimidin-4(3H)-one;
66) 2-((4-benzylphenoxy)methyl)-8-(thiazol-2-yl)pyrido[3,4-d]pyrimidin-4(3H)-one;
67) 2-((4-benzylphenoxy)methyl)-8-(pyridin-2-yl)pyrido[3,4-d]pyrimidin-4(3H)-one;
68) 8-(2-aminothiazol-5-yl)-2-((4-benzylphenoxy)methyl)pyrido[3,4-d]pyrimidin-4(3H)-one;
69) 8-(3-amino-1H-pyrazol-1-yl)-2-((4-benzylphenoxy)methyl)pyrido[3,4-d]pyrimidin-4(3H)-one;
70) 2-((4-benzylphenoxy)methyl)-8-(1-methyl-1H-imidazol-4-yl)pyrido[3,4-d]pyrimidin-4(3H)-one;
71) 8-(2-aminothiazol-4-yl)-2-((4-benzylphenoxy)methyl)pyrido[3,4-d]pyrimidin-4(3H)-one;
72) 8-(2-(allylamino)thiazol-4-yl)-2-((4-benzylphenoxy)methyl)pyrido[3,4-d]pyrimidin-4(3H)-one;
73) 2-((4-benzylphenoxy)methyl)-8-(2-(phenylamino)thiazol-4-yl)pyrido[3,4-d]pyrimidin-4(3H)-one;
74) 2-((3-chloro-4-fluorophenoxy)methyl)-8-(thiazol-2-yl)pyrido[3,4-d]pyrimidin-4(3H)-one;
75) 8-(2-aminothiazol-5-yl)-2-((3-chloro-4-fluorophenoxy)methyl)pyrido[3,4-d]pyrimidin-4(3H)-one;
76) 2-((3-chloro-4-fluorophenoxy)methyl)-8-(1H-pyrazol-3-yl)pyrido[3,4-d]pyrimidin-4(3H)-one;
77) 2-((3-chloro-4-fluorophenoxy)methyl)-8-(1-methyl-1H-imidazol-4-yl)pyrido[3,4-d]pyrimidin-4(3H) one;
78) 2-((3-chloro-4-fluorophenoxy)methyl)-8-(6-morpholinopyridin-3-yl)pyrido[3,4-d]pyrimidin-4(3H)-one;
79) 2-((3-chloro-4-fluorophenoxy)methyl)-8-(pyrazin-2-yl)pyrido[3,4-d]pyrimidin-4(3H)-one;
80) 2-((3-chloro-4-fluorophenoxy)methyl)-8-(pyridin-2-yl)pyrido[3,4-d]pyrimidin-4(3H)-one;
81) 2-((3-chloro-4-fluorophenoxy)methyl)-8-(2-((2-hydroxyethyl)amino)thiazol-4-yl)pyrido[3,4-d]pyrimidin-4(3H)-one;
82) 2-((3-chloro-4-fluorophenoxy)methyl)-8-(2-((2-morpholinoethyl)amino)thiazol-4-yl)pyrido[3,4-d]pyrimidin-4(3H)-one;
83) (R)-2-((3-chloro-4-fluorophenoxy)methyl)-8-(2-((2-hydroxypropyl)amino)thiazol-4-yl)pyrido[3,4-d]pyrimidin-4(3H)-one;
84) (S)-2-((3-chloro-4-fluorophenoxy)methyl)-8-(2-((2-hydroxypropyl)amino)thiazol-4-yl)pyrido[3,4-d]pyrimidin-4(3H)-one;
85) 2-((3-chloro-4-fluorophenoxy)methyl)-8-(2-(((tetrahydrofuran-2-yl)methyl)amino)thiazol-4-yl)pyrido[3,4-d]pyrimidin-4(3H)-one;
86) 2-((3-chloro-4-fluorophenoxy)methyl)-8-(2-((2-methoxyethyl)amino)thiazol-4-yl)pyrido[3,4-d]pyrimidin-4(3H)-one;
87) 2-((3-chloro-4-fluorophenoxy)methyl)-8-(2-((2-(dimethylamino)ethyl)amino)thiazol-4-yl)pyrido[3,4 d]pyrimidin-4(3H)-one;
88) 8-(2-((2-aminoethyl)amino)thiazol-4-yl)-2-((3-chloro-4-fluorophenoxy)methyl)pyrido[3,4-d]pyrimidin-4(3H)-one;
89) 2-((3-chloro-4-fluorophenoxy)methyl)-8-(2-((2-(4-methylpiperazin-1-yl)ethyl)amino)thiazol-4-yl)pyrido[3,4-d]pyrimidin-4(3H)-one;
90) 2-((3-chloro-4-fluorophenoxy)methyl)-8-(2-((3-hydroxypropyl)amino)thiazol-4-yl)pyrido[3,4-d]pyrimidin-4(3H)-one;
91) 2-((3-chloro-4-fluorophenoxy)methyl)-8-(2-((3-(dimethylamino)propyl)amino)thiazol-4-yl)pyrido[3,4-d]pyrimidin-4(3H)-one;
92) 2-((3-chloro-4-fluorophenoxy)methyl)-8-(2-((2-(1-methylpyrrolidin-2-yl)ethyl)amino)thiazol-4-yl)pyrido[3,4-d]pyrimidin-4(3H)-one;
93) 2-((3-chloro-4-fluorophenoxy)methyl)-8-(2-(piperidin-4-ylamino)thiazol-4-yl)pyrido[3,4-d]pyrimidin-4(3H)-one;
94) 2-((3-chloro-4-fluorophenoxy)methyl)-8-(2-((1-methylpiperidin-4-yl)amino)thiazol-4-yl)pyrido[3,4-d]pyrimidin-4(3H)-one;
95) 2-((3-chloro-4-fluorophenoxy)methyl)-8-(2-((2-hydroxybutyl)amino)thiazol-4-yl)pyrido[3,4-d]pyrimidin-4(3H)-one;
96) 8-(2-(((1-benzylpiperidin-4-yl)methyl)amino)thiazol-4-yl)-2-((3-chloro-4-fluorophenoxy)methyl)pyrido[3,4-d]pyrimidin-4(3H)-one;
97) 2-((3-chloro-4-fluorophenoxy)methyl)-8-(2-(((1-isopropylpiperidin-4-yl)methyl)amino)thiazol-4-yl)pyrido[3,4-d]pyrimidin-4(3H)-one;
98) 2-((3-chloro-4-fluorophenoxy)methyl)-8-(2-(((1-cyclopentylpiperidin-4-yl)methyl)amino)thiazol-4-yl)pyrido[3,4-d]pyrimidin-4(3H)-one;
99) 2-((3-chloro-4-fluorophenoxy)methyl)-8-(2-((piperidin-4-ylmethyl)amino)thiazol-4-yl)pyrido[3,4-d]pyrimidin-4(3H)-one;
100) 2-((3-chloro-4-fluorophenoxy)methyl)-8-(2-(((1-(2-hydroxyethyl)piperidin-4-yl)methyl)amino)thiazol-4-yl)pyrido[3,4-d]pyrimidin-4(3H)-one;
101) 2-((3-chloro-4-fluorophenoxy)methyl)-8-(2-(((1-(2-methoxyethyl)piperidin-4-yl)methyl)amino)thiazol-4-yl)pyrido[3,4-d]pyrimidin-4(3H)-one;
102) 8-(2-((1-benzylpiperidin-4-yl)amino)thiazol-4-yl)-2-((3-chloro-4-fluorophenoxy)methyl)pyrido[3,4-d]pyrimidin-4(3H)-one;
103) 2-((3-chloro-4-fluorophenoxy)methyl)-8-(2-methylthiazol-4-yl)pyrido[3,4-d]pyrimidin-4(3H)-one;
104) 8-(2-aminothiazol-4-yl)-2-((3-chloro-4-fluorophenoxy)methyl)pyrido[3,4-d]pyrimidin-4(3H)-one;
105) 2-((3-chloro-4-fluorophenoxy)methyl)-8-(2-(diethylamino)thiazol-4-yl)pyrido[3,4-d]pyrimidin-4(3H)-one;
106) 2-((3-chloro-4-fluorophenoxy)methyl)-8-(thiazol-4-yl)pyrido[3,4-d]pyrimidin-4(3H)-one;
107) 8-(2-((1-benzylpyrrolidin-3-yl)amino)thiazol-4-yl)-2-((3-chloro-4-fluorophenoxy)methyl)pyrido[3,4-d]pyrimidin-4(3H)-one;
108) 2-((3-chloro-4-fluorophenoxy)methyl)-8-(2-((3,4-dichlorophenethyl)amino)thiazol-4-yl)pyrido[3,4-d]pyrimidin-4(3H)-one;
109) 2-((3-chloro-4-fluorophenoxy)methyl)-8-(2-((3-(2-chlorophenyl)propyl)amino)thiazol-4-yl)pyrido[3,4-d]pyrimidin-4(3H)-one;
110) 2-((3-chloro-4-fluorophenoxy)methyl)-8-(2-((2-phenoxyphenethyl)amino)thiazol-4-yl)pyrido[3,4-d]pyrimidin-4(3H)-one;
111) 2-((3-chloro-4-fluorophenoxy)methyl)-8-(2-((2-hydroxy-2-(pyridin-4-yl)ethyl)amino)thiazol-4-yl)pyrido[3,4-d]pyrimidin-4(3H)-one;
112) 2-((3-chloro-4-fluorophenoxy)methyl)-8-(2-((3,4-dimethylphenethyl)amino)thiazol-4-yl)pyrido[3,4-d]pyrimidin-4(3H)-one;
113) 2-((3-chloro-4-fluorophenoxy)methyl)-8-(2-((2-hydroxy-3-(piperazin-1-yl)propyl)amino)thiazol-4-yl)pyrido[3,4-d]pyrimidin-4(3H)-one;
114) 2-((3-chloro-4-fluorophenoxy)methyl)-8-(6-chloropyridin-2-yl)pyrido[3,4-d]pyrimidin-4(3H)-one;
115) 2-((3-chloro-4-fluorophenoxy)methyl)-8-(6-(4-methylpiperidin-1-yl)pyridin-2-yl)pyrido[3,4-d]pyrimidin-4(3H)-one;
116) 2-((3-chloro-4-fluorophenoxy)methyl)-8-(6-morpholinopyridin-2-yl)pyrido[3,4-d]pyrimidin-4(3H)-one;
117) 2-((3-chloro-4-fluorophenoxy)methyl)-8-(2-((cyclohexylmethyl)amino)thiazol-4-yl)pyrido[3,4-d]pyrimidin-4(3H)-one;
118) 2-((3-chloro-4-fluorophenoxy)methyl)-8-(2-((5-(diethylamino)pentan-2-yl)amino)thiazol-4-yl)pyrido[3,4-d]pyrimidin-4(3H)-one;
119) 2-((3-chloro-4-fluorophenoxy)methyl)-8-(2-((4-(pyrrolidin-1-yl)butyl)amino)thiazol-4-yl)pyrido[3,4-d]pyrimidin-4(3H)-one;
120) 2-((3-chloro-4-fluorophenoxy)methyl)-8-(2-((5-(diisopropylamino)pentyl)amino)thiazol-4-yl)pyrido[3,4-d]pyrimidin-4(3H)-one;
121) (R)-2-((3-chloro-4-fluorophenoxy)methyl)-8-(2-((1-hydroxypentan-2-yl)amino)thiazol-4-yl)pyrido[3,4-d]pyrimidin-4(3H)-one;
122) ethyl 4-(2-((3-chloro-4-fluorophenoxy)methyl)-4-oxo-3,4-dihydropyrido[3,4-d]pyrimidin-8-yl)thiazole-2-carboxylate;
123) 2-((3-chloro-4-fluorophenoxy)methyl)-8-(2-((2-phenoxypropyl)amino)thiazol-4-yl)pyrido[3,4-d]pyrimidin-4(3H)-one;
124) N-(4-(2-((3-chloro-4-fluorophenoxy)methyl)-4-oxo-3,4-dihydropyrido[3,4-d]pyrimidin-8-yl)thiazol-2-yl)benzamide;
125) 2-((4-fluorophenoxy)methyl)-8-(2-((2-hydroxyethyl)amino)thiazol-4-yl)pyrido[3,4-d]pyrimidin-4(3H)-one;
126) 8-(2-((2-(dimethylamino)ethyl)amino)thiazol-4-yl)-2-((4-fluorophenoxy)methyl)pyrido[3,4-d]pyrimidin-4(3H)-one;
127) 2-((4-fluorophenoxy)methyl)-8-(2-((1-methylpiperidin-4-yl)amino)thiazol-4-yl)pyrido[3,4-d]pyrimidin-4(3H)-one;
128) 8-(2-((1-benzylpiperidin-4-yl)amino)thiazol-4-yl)-2-((4-fluorophenoxy)methyl)pyrido[3,4-d]pyrimidin-4(3H)-one;
129) 2-((3,5-difluorophenoxy)methyl)-8-(2-((2-hydroxyethyl)amino)thiazol-4-yl)pyrido[3,4-d]pyrimidin-4(3H)-one;
130) 2-((3,5-difluorophenoxy)methyl)-8-(2-((2-(dimethylamino)ethyl)amino)thiazol-4-yl)pyrido[3,4-d]pyrimidin-4(3H)-one;
131) 2-((3,5-difluorophenoxy)methyl)-8-(2-((1-methylpiperidin-4-yl)amino)thiazol-4-yl)pyrido[3,4-d]pyrimidin-4(3H)-one;
132) 2-((2-chlorophenoxy)methyl)-8-(2-((2-hydroxyethyl)amino)thiazol-4-yl)pyrido[3,4-d]pyrimidin-4(3H)-one;
133) 2-((3-chlorophenoxy)methyl)-8-(2-((2-hydroxyethyl)amino)thiazol-4-yl)pyrido[3,4-d]pyrimidin-4(3H)-one;
134) 2-((3,5-dichlorophenoxy)methyl)-8-(2-((2-hydroxyethyl)amino)thiazol-4-yl)pyrido[3,4-d]pyrimidin-4(3H)-one;
135) 2-((2,4-dichlorophenoxy)methyl)-8-(2-((2-hydroxyethyl)amino)thiazol-4-yl)pyrido[3,4-d]pyrimidin-4(3H)-one;
136) 2-((3,4-dichlorophenoxy)methyl)-8-(2-((2-hydroxyethyl)amino)thiazol-4-yl)pyrido[3,4-d]pyrimidin-4(3H)-one;
137) 8-(2-((2-hydroxyethyl)amino)thiazol-4-yl)-2-methylpyrido[3,4-d]pyrimidin-4(3H)-one;
138) 8-(2-((2-hydroxyethyl)amino)thiazol-4-yl)-2-isopropylpyrido[3,4-d]pyrimidin-4(3H)-one;
139) 2-benzyl-8-(2-((2-hydroxyethyl)amino)thiazol-4-yl)pyrido[3,4-d]pyrimidin-4(3H)-one;
140) 2-benzyl-8-(2-((2-(dimethylamino)ethyl)amino)thiazol-4-yl)pyrido[3,4-d]pyrimidin-4(3H)-one;
141) 8-(2-((2-hydroxyethyl)amino)thiazol-4-yl)-2-(1-phenylethyl)pyrido[3,4-d]pyrimidin-4(3H)-one;
142) 2-((benzyloxy)methyl)-8-(2-((2-hydroxyethyl)amino)thiazol-4-yl)pyrido[3,4-d]pyrimidin-4(3H)-one;
143) 2-((benzyloxy)methyl)-8-(2-((2-hydroxybutyl)amino)thiazol-4-yl)pyrido[3,4-d]pyrimidin-4(3H)-one;
144) 2-((benzyloxy)methyl)-8-(2-((1-benzylpiperidin-4-yl)amino)thiazol-4-yl)pyrido[3,4-d]pyrimidin-4(3H)-one;
145) 2-((benzyloxy)methyl)-8-(2-(((1-benzylpiperidin-4-yl)methyl)amino)thiazol-4-yl)pyrido[3,4-d]pyrimidin-4(3H)-one;
146) 2-((benzyloxy)methyl)-8-(2-((2-methoxyethyl)amino)thiazol-4-yl)pyrido[3,4-d]pyrimidin-4(3H)-one;
147) 2-((benzyloxy)methyl)-8-(2-((3-hydroxypropyl)amino)thiazol-4-yl)pyrido[3,4-d]pyrimidin-4(3H)-one;
148) 2-((benzyloxy)methyl)-8-(2-((2-(dimethylamino)ethyl)amino)thiazol-4-yl)pyrido[3,4-d]pyrimidin-4(3H)-one;
149) 8-(2-((2-aminoethyl)amino)thiazol-4-yl)-2-((benzyloxy)methyl)pyrido[3,4-d]pyrimidin-4(3H)-one;
150) 2-((benzyloxy)methyl)-8-(2-((3-(dimethylamino)propyl)amino)thiazol-4-yl)pyrido[3,4-d]pyrimidin-4(3H)-one;
151) 2-((benzyloxy)methyl)-8-(2-((2-(4-methylpiperazin-1-yl)ethyl)amino)thiazol-4-yl)pyrido[3,4-d]pyrimidin-4(3H)-one;
152) 2-((benzyloxy)methyl)-8-(2-((2-(1-methylpyrrolidin-2-yl)ethyl)amino)thiazol-4-yl)pyrido[3,4-d]pyrimidin-4(3H)-one;
153) 2-((benzyloxy)methyl)-8-(2-(((1-(2-hydroxyethyl)piperidin-4-yl)methyl)amino)thiazol-4-yl)pyrido[3,4-d]pyrimidin-4(3H)-one;
154) 2-((benzyloxy)methyl)-8-(2-(((1-(2-methoxyethyl)piperidin-4-yl)methyl)amino)thiazol-4-yl)pyrido[3,4-d]pyrimidin-4(3H)-one;
155) 2-((benzyloxy)methyl)-8-(2-((1-methylpiperidin-4-yl)amino)thiazol-4-yl)pyrido[3,4-d]pyrimidin-4(3H)-one;
156) 2-(([1,1′-biphenyl]-3-yloxy)methyl)-8-(2-((2-hydroxyethyl)amino)thiazol-4-yl)pyrido[3,4-d]pyrimidin-4(3H)-one;
157) 8-(2-((2-hydroxyethyl)amino)thiazol-4-yl)-2-((4-phenoxyphenoxy)methyl)pyrido[3,4-d]pyrimidin-4(3H)-one;
158) 8-(2-((2-hydroxyethyl)amino)thiazol-4-yl)-2-((4-(2-phenylpropan-2-yl)phenoxy)methyl)pyrido[3,4-d]pyrimidin-4(3H)-one;
159) 2-((4-cyclopentylphenoxy)methyl)-8-(2-((2-hydroxyethyl)amino)thiazol-4-yl)pyrido[3,4-d]pyrimidin-4(3H)-one;
160) 2-((4-benzoylphenoxy)methyl)-8-(2-((2-hydroxyethyl)amino)thiazol-4-yl)pyrido[3,4-d]pyrimidin-4(3H)-one;
161) 8-(2-((2-hydroxyethyl)amino)thiazol-4-yl)-2-phenethylpyrido[3,4-d]pyrimidin-4(3H)-one;
162) 8-(2-((2-(dimethylamino)ethyl)amino)thiazol-4-yl)-2-phenethylpyrido[3,4-d]pyrimidin-4(3H)-one;
163) 8-(2-((1-methylpiperidin-4-yl)amino)thiazol-4-yl)-2-phenethylpyrido[3,4-d]pyrimidin-4(3H)-one;
164) 8-(2-((1-benzylpiperidin-4-yl)amino)thiazol-4-yl)-2-phenethylpyrido[3,4-d]pyrimidin-4(3H)-one;
165) 8-(2-aminothiazol-4-yl)-2-(4-fluorophenethyl)pyrido[3,4-d]pyrimidin-4(3H)-one;
166) 8-(2-aminothiazol-4-yl)-2-(3-phenylpropyl)pyrido[3,4-d]pyrimidin-4(3H)-one;
167) 2-(benzofuran-2-yl)-8-(2-((2-hydroxyethyl)amino)thiazol-4-yl)pyrido[3,4-d]pyrimidin-4(3H)-one;
168) (S)-2-(benzofuran-2-yl)-8-(2-((2-hydroxypropyl)amino)thiazol-4-yl)pyrido[3,4-d]pyrimidin-4(3H)-one;
169) (E)-8-(2-((2-hydroxyethyl)amino)thiazol-4-yl)-2-styrylpyrido[3,4-d]pyrimidin-4(3H)-one;
170) methyl 8-(2-((2-hydroxyethyl)amino)thiazol-4-yl)-4-oxo-3,4-dihydropyrido[3,4-d]pyrimidine-2-carboxylate;
171) 2-((3-fluorophenoxy)methyl)-8-(2-((2-hydroxyethyl)amino)thiazol-4-yl)pyrido[3,4-d]pyrimidin-4(3H)-one;
172) 2-((4-chlorophenoxy)methyl)-8-(2-((2-hydroxyethyl)amino)thiazol-4-yl)pyrido[3,4-d]pyrimidin-4(3H)-one 173) 2-((3,4-difluorophenoxy)methyl)-8-(2-((2-hydroxyethyl)amino)thiazol-4-yl)pyrido[3,4-d]pyrimidin-4(3H)-one;
174) 2-((4-chloro-3-fluorophenoxy)methyl)-8-(2-((2-hydroxyethyl)amino)thiazol-4-yl)pyrido[3,4-d]pyrimidin-4(3H)-one;
175) 2-((4-benzylphenoxy)methyl)-8-(2-((2-hydroxyethyl)amino)thiazol-4-yl)pyrido[3,4-d]pyrimidin-4(3H)-one;
176) 8-(2-((2-hydroxyethyl)amino)thiazol-4-yl)-4-oxo-3,4-dihydropyrido[3,4-d]pyrimidine-2-carboxylic acid;
177) 2-((3-chloro-4-fluorophenoxy)methyl)-8-(2-(((4-methylmorpholin-2-yl)methyl)amino)thiazol-4-yl)pyrido[3,4-d]pyrimidin-4(3H)-one;
178) 2-((3-chloro-4-fluorophenoxy)methyl)-8-(2-(((1,3-dihydroxypropan-2-yl)amino)thiazol-4-yl)pyrido[3,4-d]pyrimidin-4(3H)-one;
179) 2-((3-chloro-4-fluorophenoxy)methyl)-8-(2-((2,3-dihydroxypropyl)amino)thiazol-4-yl)pyrido[3,4-d]pyrimidin-4(3H)-one;
180) 2-((3-chloro-4-fluorophenoxy)methyl)-8-(2-((tetrahydro-2H-pyran-3-yl)amino)thiazol-4-yl)pyrido[3,4-d]pyrimidin-4(3H)-one;
181) 2-((3-chloro-4-fluorophenoxy)methyl)-8-(2-((4-hydroxyphenyl)amino)thiazol-4-yl)pyrido[3,4-d]pyrimidin-4(3H)-one;
182) 2-((3,4-dimethylphenoxy)methyl)-8-(2-((2-hydroxyethyl)amino)thiazol-4-yl)pyrido[3,4-d]pyrimidin-4(3H)-one;
183) 2-((3-benzylphenoxy)methyl)-8-(2-((2-hydroxyethyl)amino)thiazol-4-yl)pyrido[3,4-d]pyrimidin-4(3H)-one;
184) 2-((4-chloro-3-methylphenoxy)methyl)-8-(2-((2-hydroxyethyl)amino)thiazol-4-yl)pyrido[3,4-d]pyrimidin-4(3H)-one;
185) 8-(2-((2-hydroxyethyl)amino)thiazol-4-yl)-2-((4-(trifluoromethyl)phenoxy)methyl)pyrido[3,4-d]pyrimidin-4(3H)-one;
186) 8-(2-((2-hydroxyethyl)amino)thiazol-4-yl)-2-((4-methoxyphenoxy)methyl)pyrido[3,4-d]pyrimidin-4(3H)-one;
187) 2-((2-chloro-4-fluorophenoxy)methyl)-8-(2-((2-hydroxyethyl)amino)thiazol-4-yl)pyrido[3,4-d]pyrimidin-4(3H)-one;
188) 8-(2-aminothiazol-4-yl)-2-benzylpyrido[3,4-d]pyrimidin-4(3H)-one;
189) 2-benzyl-8-(2-((1-methylpiperidin-4-yl)amino)thiazol-4-yl)pyrido[3,4-d]pyrimidin-4(3H)-one; and
190) 2-benzyl-8-(2-((piperidin-4-ylmethyl)amino)thiazol-4-yl)pyrido[3,4-d]pyrimidin-4(3H)-one.

6. A pharmaceutical composition comprising the compound of claim 1, the isomer thereof, or the pharmaceutically acceptable salt thereof, as an effective ingredient.

7. The pharmaceutical composition of claim 6, wherein it is for prevention or treatment of a cancer or a disease related to KDM dysregulation.

8. The pharmaceutical composition of claim 6, wherein the cancer is selected from the group consisting of prostate cancer, lung cancer, breast cancer, gastric cancer, cervical cancer, melanoma, renal cell carcinoma and leukemia.

9. A method for preventing or treating a cancer or a disease related to KDM dysregulation comprising administering the compound of claim 1.

10. The method of claim 9, wherein the cancer is selected from the group consisting of prostate cancer, lung cancer, breast cancer, gastric cancer, cervical cancer, melanoma, renal cell carcinoma and leukemia.

11. A use of the compound of claim 1 for preventing or treating a cancer or a disease related to KDM dysregulation.

12. The use of claim 11, wherein the cancer is selected from the group consisting of prostate cancer, lung cancer, breast cancer, gastric cancer, cervical cancer, melanoma, renal cell carcinoma and leukemia.
